(19) United States
(12) Patent Application Publication
(10) Pub. No.: US 2025/0195464 A1
(43) Pub. Date: Jun. 19, 2025

(54) ALOTAKETAL COMPOUNDS AND DERIVATIVES THEREOF FOR USE AS ANTIVIRAL AGENTS

(71) Applicant: THE UNIVERSITY OF BRITISH COLUMBIA, Vancouver (CA)

(72) Inventors: Raymond ANDERSEN, Vancouver (CA); François JEAN, Vancouver (CA); David WILLIAMS, Vancouver (CA); Polina BLAGOJEVIC, Vancouver (CA); Jimena PEREZ VARGAS, Vancouver (CA)

(73) Assignee: THE UNIVERSITY OF BRITISH COLUMBIA, Vancouver, BC (CA)

(21) Appl. No.: 18/846,525

(22) PCT Filed: Mar. 17, 2023
(86) PCT No.: PCT/CA2023/050358

§ 371 Date: Sep. 12, 2024

Related U.S. Application Data
(60) Provisional application No. 63/321,375, filed on Mar. 18, 2022.

(51) Int. Cl. A61K 31/352; A61P 31/14

(52) U.S. Cl. CPC A61K 31/352; A61P 31/14

(57) ABSTRACT

Provided herein are Alotaketal compounds and derivatives thereof that have antiviral activity. In particular, the invention relates to a subset of compounds represented by Formulas (1), (2) and (3), for use as antiviral agents in the treatment or prevention of coronavirus infection. Methods for using the compounds in the treatment or prophylaxis of a coronavirus infection are provided. In particular, the coronavirus infection may selected from one or more of the following: Severe Acute Respiratory Syndrome (SARS) coronavirus-1 (SARS-C0V-1) infection; SARS coronavirus-2 (SARS-C0V-2) infection; and Middle East Respiratory Syndrome (MERS) coronavirus (MERS-CoV) infection. More specifically, the coronavirus infection may be a human coronavirus 229E (HC0V-229E) infection.

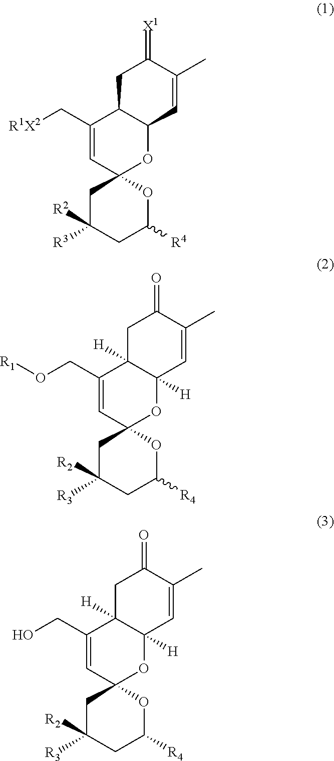

### CROSS REFERENCE TO RELATED APPLICATIONS

This application claims the benefit of U.S. Provisional Patent Application Ser. No. 63/321,375 filed 18 Mar. 2022 entitled “COMPOUNDS FOR REDUCING THE ACTIVITY OF CORONAVIRUSES”.

### TECHNICAL FIELD

The present invention relates to Alotaketal compounds and derivatives thereof having antiviral activity. In particular, the invention relates to a subset of compounds represented by Formulas 1, 2 and 3, for use as antiviral agents in the treatment or prevention of coronavirus infection. Methods for using the compounds in the treatment or prophylaxis of a coronavirus infection are provided. In particular, the coronavirus infection may be selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection. More specifically, the coronavirus infection may be a human coronavirus 229E (HCoV-229E) infection.

### BACKGROUND

The severe acute respiratory syndrome coronavirus 2 (SARS-CoV-2) is responsible for the ongoing COVID-19 pandemic, a major global challenge to public health (McKee M 2020). As of 17 Mar. 2022, more than 464 million SARS-CoV-2 infections and over 6 million deaths have been reported (CSSE—Johns Hopkins). Multiple vaccines have been authorized and distributed worldwide, along with other public health measures. However, several new variants have emerged, presenting a higher capacity of transmission, infectivity, and increasing prevalence worldwide, and having the potential to reduce the effectiveness of vaccines. SARS-CoV-2 BQ.1.1 and XBB.1.5 subvariants of BA.5 and BA.2, respectively, are presently the most prevalent variants of concern (VOCs), with greater transmissibility and increased risk of reinfection, even in populations with high vaccination rates (Miller 2023). The constant evolution of variants, enhance the need to development of prophylactic and therapeutic effective solutions with broad activity against the VOCs (Wibmer 2021).

Natural products (NPs) are a promising, but undervalued resource for new antivirals. Compounds derived from diverse sources can encompass structural diversity that falls outside the scope of chemical spaces found in synthetic chemical compounds, they have the potential to act via a mechanism distinct from those of conventional therapies. NP have been highlighted for their antiviral potential against a variety of viruses, with potential novel inhibitors and activators of biological pathways, able of inhibiting SARS-CoV-2 infections.

### SUMMARY

The present invention is based, in part, on the surprising discovery that a subset of compounds represented by Formulas 1, 2 and 3 described herein are useful as antiviral agents in the treatment or prevention of coronavirus infection. In particular, it is shown herein that compounds having R⁴ chirality

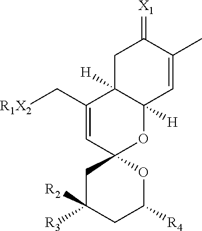

had greater potency, than those compounds with no alkyl group at R⁴ or the opposite chirality. Methods for using the compounds in the treatment or prophylaxis of a coronavirus infection are provided. In particular, the coronavirus infection may be selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection. More specifically, the coronavirus infection may be a human coronavirus 229E (HCoV-229E) infection.

In a first embodiment, there is provided a compound, the compound may have the structure of Formula 1:

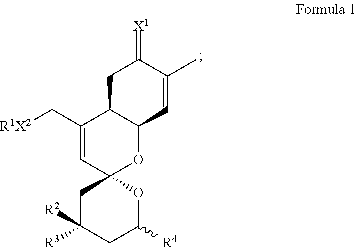

or pharmaceutical acceptable salt or prodrug thereof, for treatment of a coronavirus infection, wherein: X¹ may be selected from: O; S; N—OH; and N—NH—R⁵; X² may be selected from: O; and S; R¹ may be selected from: H; and a one to ten carbon saturated, or unsaturated, linear, branched, or cyclic alkyl group, where individual carbon atoms may be optionally substituted with OH, OR, ═O, NH, SH, F, Cl, Br, or may be optionally replaced by N, or S; R² and R³ may be independently selected from: H; R; OR; or R₂ and R₃ jointly form ═CR⁶R⁷; R⁴ may be selected from: H; and a one to twenty carbon saturated, unsaturated, or aromatic, linear, branched, or cyclic alkyl group, where individual carbon atoms may be optionally substituted with O, OH, OR, ═O, NH, SH, F, Cl, Br, or may be optionally replaced by N, or S; R⁵ may be selected from: H; and a one to ten carbon saturated, or unsaturated, linear, branched, or cyclic alkyl group, where individual carbon atoms may be optionally substituted with OH, OR, ═O, NH, SH, F, Cl, Br, or may be optionally replaced by N, or S; R⁶ may be selected from: H; and a one to ten carbon saturated, or unsaturated, linear, branched, or cyclic, alkyl group where individual carbon atoms may be optionally substituted with OH, OR, ═O, NH, SH, F, Cl, Br, or may be optionally replaced by N, or S; R⁷ may be selected from: H; and a one to ten carbon saturated, or unsaturated, linear, branched, or cyclic alkyl group where individual carbon atoms may be optionally substituted with OH, OR, ═O, NH, SH, F, Cl, Br, or may be optionally replaced by N, or S; and R may be selected from: C(═O)CH₃; C(═O)CH₂CH(CH₃)CH₃; H; and CH₃.

In a further embodiment, there is provided a compound, the compound having the structure of Formula 3:

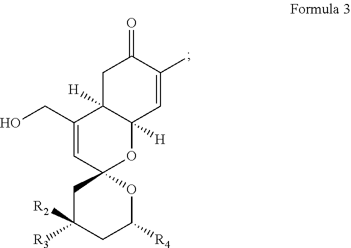

wherein: R² is selected from: H; CH₃; OC(═O)CH₃; and OC(═O)CH₂CH(CH₃)CH₃; R₃ is selected from: H; CH₃; OC(═O)CH₃; and OC(═O)CH₂CH(CH₃)CH₃; alternatively, R² and R³ jointly form ═CH₂; and R⁴ is a one to twenty carbon saturated, unsaturated, or aromatic, linear, branched, or cyclic alkyl group where individual carbon atoms may be substituted with OH, ═O, NH, SH, F, Cl, Br, or be replaced by N, or S; provided that the compound excludes the following:

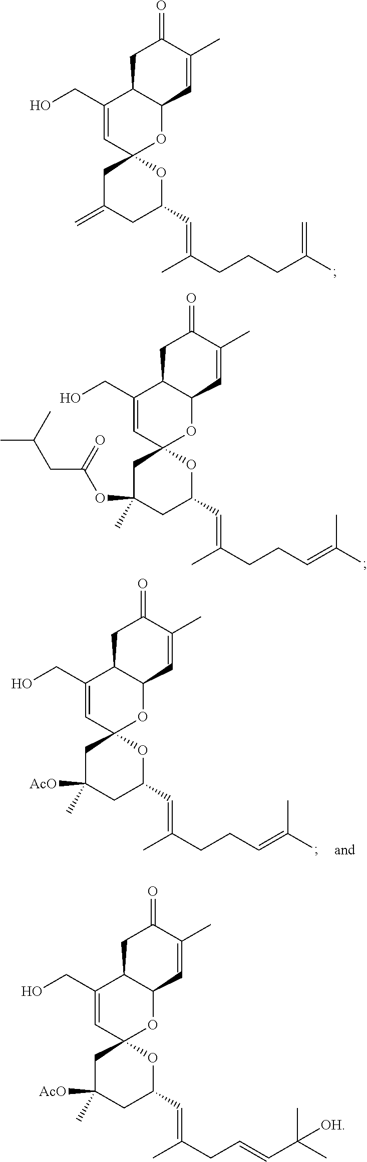

In a further embodiment, there is provided a method of treating a coronavirus infection in a subject in need thereof, the method including administration of a compound to the subject, wherein the compound is a compound described herein, or pharmaceutical acceptable salt or prodrug thereof.

In a further embodiment, there is provided a pharmaceutical composition, the pharmaceutical composition including a compound described herein, or pharmaceutical acceptable salt or prodrug thereof, for treatment of a coronavirus infection.

In a further embodiment, there is provided a pharmaceutical composition, the pharmaceutical composition including (a) a compound of Formula 3 as described herein or pharmaceutical acceptable salt or prodrug thereof, and (b) a pharmaceutically acceptable carrier.

In a further embodiment, there is provided a use of a compound, or pharmaceutical acceptable salt or prodrug thereof, or a pharmaceutical composition as described herein, for treating a coronavirus infection

In a further embodiment, there is provided a use of a compound, or pharmaceutical acceptable salt or prodrug thereof, or a pharmaceutical composition as described herein, in the manufacture of a medicament for treating a coronavirus infection.

Alternatively, the compound may have the structure

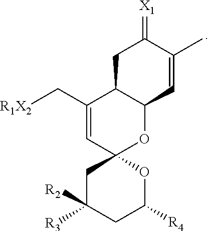

Alternatively, R⁴ may be a saturated, unsaturated, linear or branched C8 to C12 alkyl group.

Alternatively, R⁴ may be selected from: H;

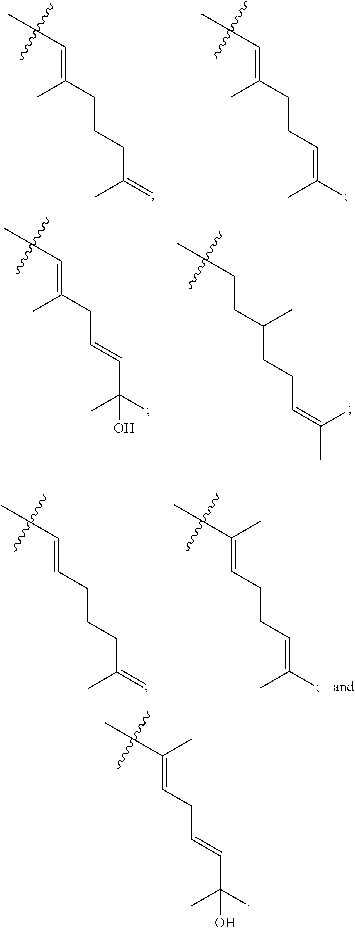

Alternatively, R⁴ may be selected from: H;

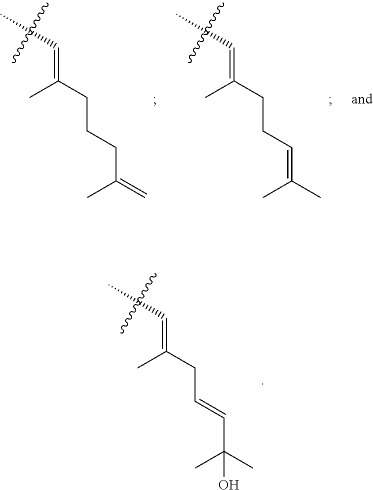

Alternatively, R⁴ may be selected from: H;

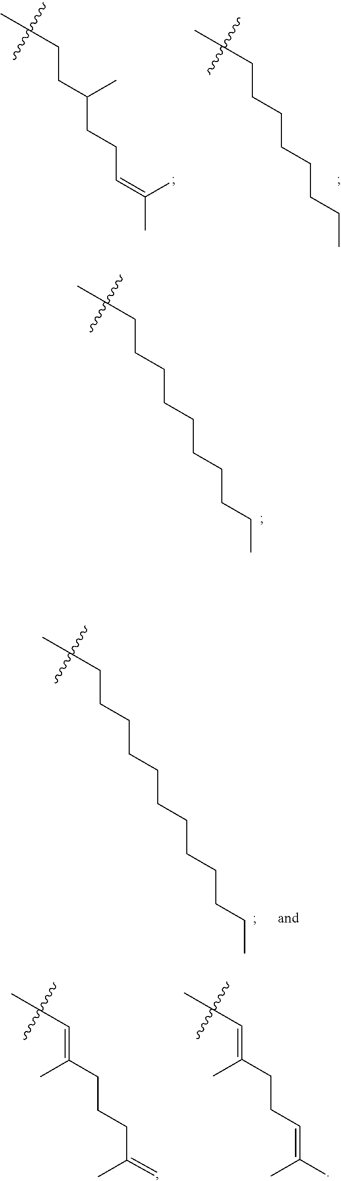

Alternatively, R⁴ may be selected from: H;

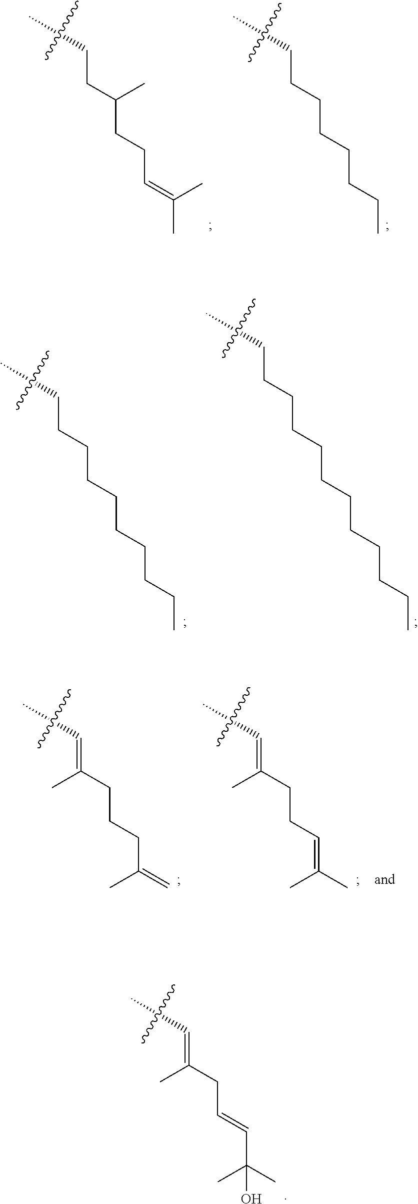

Alternatively, R⁴ may be selected from:

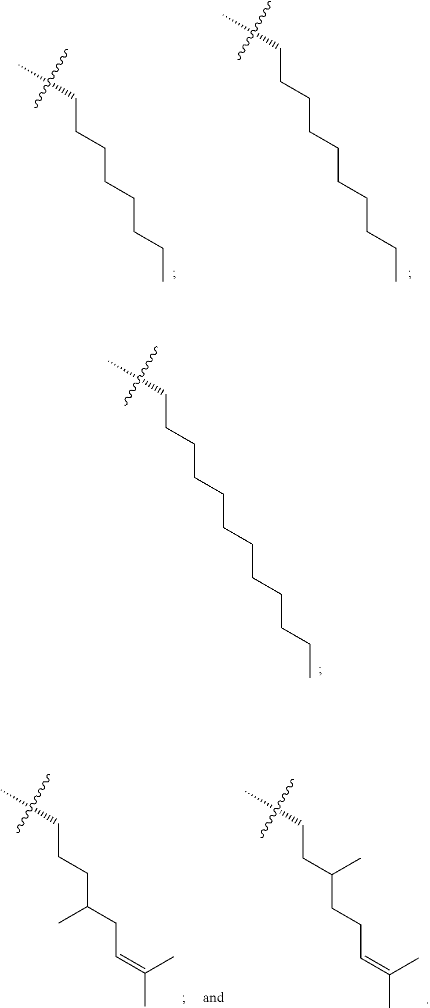

Alternatively, R⁴ may be selected from:

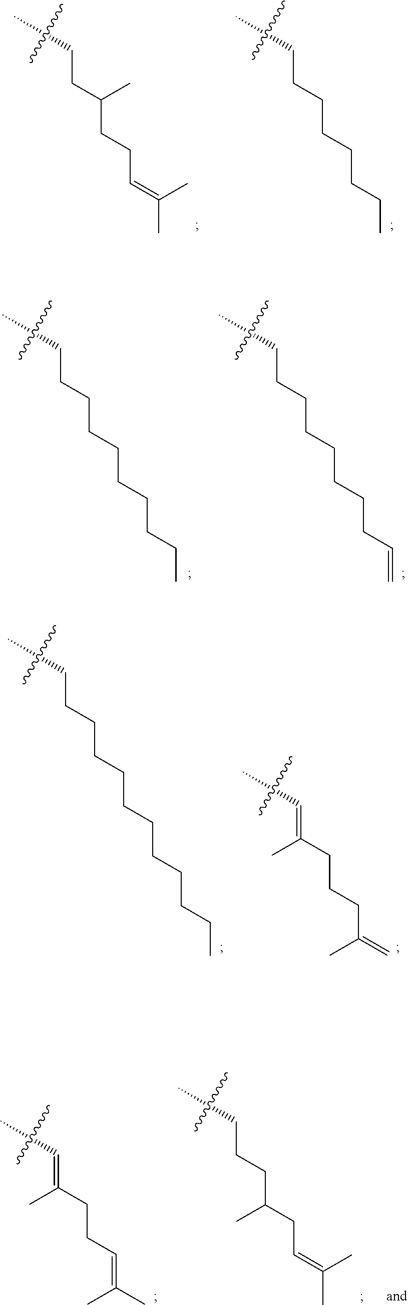

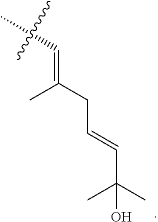

Alternatively, X¹ may be selected from: O; S; N—OH; and N—NH—R⁵. Alternatively, X¹ may be selected from: O; S; and N—OH. Alternatively, X¹ may be selected from: O; and S. Alternatively, X² may be selected from: O; and S. Alternatively, X² may be O. Alternatively, X² may be S. Alternatively, R¹ may be selected from: H; and a one to ten carbon saturated, or unsaturated, linear, branched, or cyclic alkyl group, where individual carbon atoms may be optionally substituted with OH, OR, ═O, NH, SH, F, Cl, Br, or may be optionally replaced by N, or S. Alternatively, R¹ may be H. Alternatively, R¹ may be a one to ten carbon saturated, or unsaturated, linear, branched, or cyclic alkyl group, where individual carbon atoms may be optionally substituted with OH, OR, ═O, NH, SH, F, Cl, Br, or may be optionally replaced by N, or S. Alternatively, R¹ may be a one to ten carbon saturated, or unsaturated, linear, branched, or cyclic alkyl group, where individual carbon atoms may be optionally substituted with OH, OR, ═O, NH, SH, F, Cl, Br. Alternatively, R¹ may be a one to ten carbon saturated, or unsaturated, linear, branched, or cyclic alkyl group, where individual carbon atoms or may be optionally replaced by N, or S. Alternatively, R² and R³ may be independently selected from: H; R; OR; or R₂ and R₃ may jointly form ═CR⁶R⁷. Alternatively, R² and R³ may be independently selected from: H; R; OR. Alternatively, R₂ and R₃ may jointly form ═CR⁶R⁷. Alternatively, R² and R³ may be independently selected from: H; or R. Alternatively, R² and R³ may be: H; or R. Alternatively, R² and R³ may be H. Alternatively, R² and R³ may be R.

Alternatively, R⁴ may be selected from: H; and a one to twenty carbon saturated, unsaturated, or aromatic, linear, branched, or cyclic alkyl group, where individual carbon atoms may be optionally substituted with OH, OR, ═O, NH, SH, F, Cl, Br, or may be optionally replaced by N, or S. Alternatively, R⁴ may be selected from: H. Alternatively, R⁴ may be a one to twenty carbon saturated, unsaturated, or aromatic, linear, branched, or cyclic alkyl group, where individual carbon atoms may be optionally substituted with OH, OR, ═O, NH, SH, F, Cl, Br, or may be optionally replaced by N, or S. Alternatively, R⁴ may be a one to twenty carbon saturated, unsaturated, or aromatic, linear, branched, or cyclic alkyl group, where individual carbon atoms may be optionally substituted with OH, OR, ═O, NH, SH, F, Cl, and Br. Alternatively, R⁴ may be a one to twenty carbon saturated, unsaturated, or aromatic, linear, branched, or cyclic alkyl group, where individual carbon atoms may be optionally substituted with OH, ═O, NH, SH, F, Cl, and Br. Alternatively, R⁵ may be selected from: H; and a one to ten carbon saturated, or unsaturated, linear, branched, or cyclic alkyl group, where individual carbon atoms may be optionally substituted with OH, OR, ═O, NH, SH, F, Cl, Br, or may be optionally replaced by N, or S. Alternatively, R⁵ may be H. Alternatively, R⁵ may be a one to ten carbon saturated, or unsaturated, linear, branched, or cyclic alkyl group, where individual carbon atoms may be optionally substituted with OH, OR, ═O, NH, SH, F, Cl, and Br. Alternatively, R⁵ may be selected from: H; and a one to ten carbon saturated, or unsaturated, linear, branched, or cyclic alkyl group, where individual carbon atoms may be optionally substituted with OH, OR, ═O, NH, SH, F, Cl, and Br. Alternatively, R⁶ may be selected from: H; and a one to ten carbon saturated, or unsaturated, linear, branched, or cyclic alkyl group, where individual carbon atoms may be optionally substituted with OH, OR, ═O, NH, SH, F, Cl, Br, or may be optionally replaced by N, or S. Alternatively, R⁶ may be H. Alternatively, R⁶ may be a one to ten carbon saturated, or unsaturated, linear, branched, or cyclic alkyl group, where individual carbon atoms may be optionally substituted with OH, OR, ═O, NH, SH, F, Cl, and Br. Alternatively, R⁶ may be selected from: H; and a one to ten carbon saturated, or unsaturated, linear, branched, or cyclic alkyl group, where individual carbon atoms may be optionally substituted with OH, OR, ═O, NH, SH, F, Cl, and Br. Alternatively, R⁷ may be selected from: H; and a one to ten carbon saturated, or unsaturated, linear, branched, or cyclic alkyl group, where individual carbon atoms may be optionally substituted with OH, OR, ═O, NH, SH, F, Cl, Br, or may be optionally replaced by N, or S. Alternatively, R⁷ may be H. Alternatively, R⁷ may be a one to ten carbon saturated, or unsaturated, linear, branched, or cyclic alkyl group, where individual carbon atoms may be optionally substituted with OH, OR, ═O, NH, SH, F, Cl, and Br. Alternatively, R⁷ may be selected from: H; and a one to ten carbon saturated, or unsaturated, linear, branched, or cyclic alkyl group, where individual carbon atoms may be optionally substituted with OH, OR, ═O, NH, SH, F, Cl, and Br. Alternatively, R may be selected from: C(═O)CH₃; C(═O)CH₂CH(CH₃)CH₃; H; and CH₃. Alternatively, R may be selected from: H; and CH₃. Alternatively, R may be selected from: C(═O)CH₃; H; and CH₃. Alternatively, R may be selected from: C(═O)CH₂CH(CH₃)CH₃; H; and CH₃. Alternatively, R may be selected from: C(═O)CH₃; and C(═O)CH₂CH(CH₃)CH₃. Alternatively, R may be selected from: C(═O)CH₃; C(═O)CH₂CH(CH₃)CH₃; and CH₃. Alternatively, R may be selected from: C(═O)CH₃; C(═O)CH₂CH(CH₃)CH₃; and H. Alternatively, R may be selected from: C(═O)CH₃; and CH₃. Alternatively, R may be selected from: C(═O)CH₂CH(CH₃)CH₃; and CH₃. Alternatively, R may be C(═O)CH₃. Alternatively, R may be C(═O)CH₂CH(CH₃)CH₃. Alternatively, R may be H. Alternatively, R may be CH₃. Alternatively, R² and R³ may be independently selected from: H; OC(═O)CH₃; OC(═O)CH₂CH(CH₃)CH₃; and OH; or jointly are ═CH₂. Alternatively, X¹ may be selected from: O; and S; and X² is O. Alternatively, R¹ may be H. Alternatively, compound may have the structure of Formula 2:

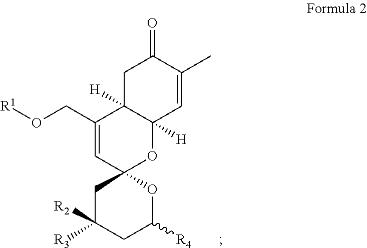

wherein: R¹ may be selected from: H; and CH₃C(O); R² may be OC(═O)CH₃; and R₃ is CH₃; or R² and R³ may jointly form ═CH₂; R⁴ may be selected from: H; and a one to twenty carbon saturated, unsaturated, or aromatic, linear, branched, or cyclic alkyl group where individual carbon atoms may be substituted with O, OH, OR, ═O, NH, SH, F, Cl, Br, or be replaced by N, or S; and R may be selected from: C(═O)CH₃; C(═O)CH₂CH(CH₃)CH₃; H; and CH₃. Alternatively, the compound may have the structure

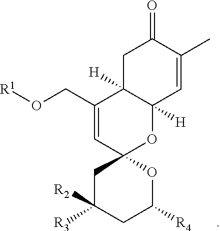

Alternatively, R⁴ may be a saturated, unsaturated, linear or branched C8 to C12 alkyl group. Alternatively, R⁴ may be selected from: H;

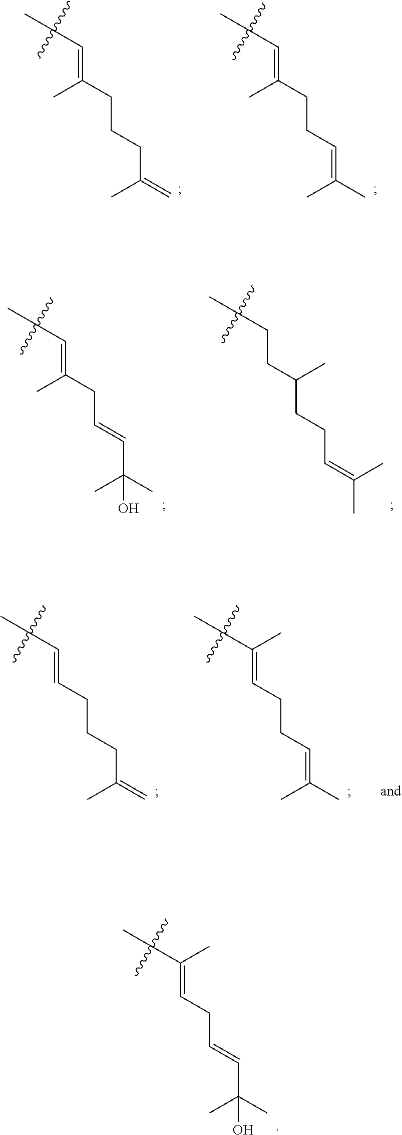

Alternatively, R⁴ may be selected from: H;

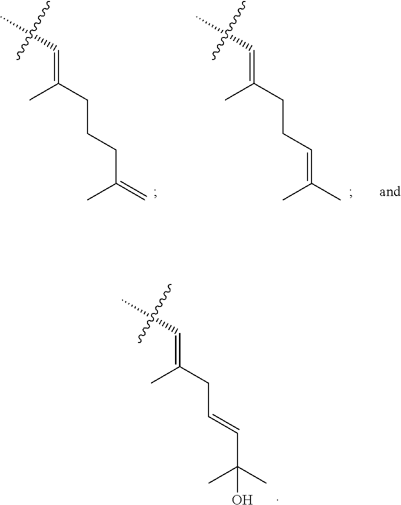

Alternatively, R⁴ may be selected from: H;

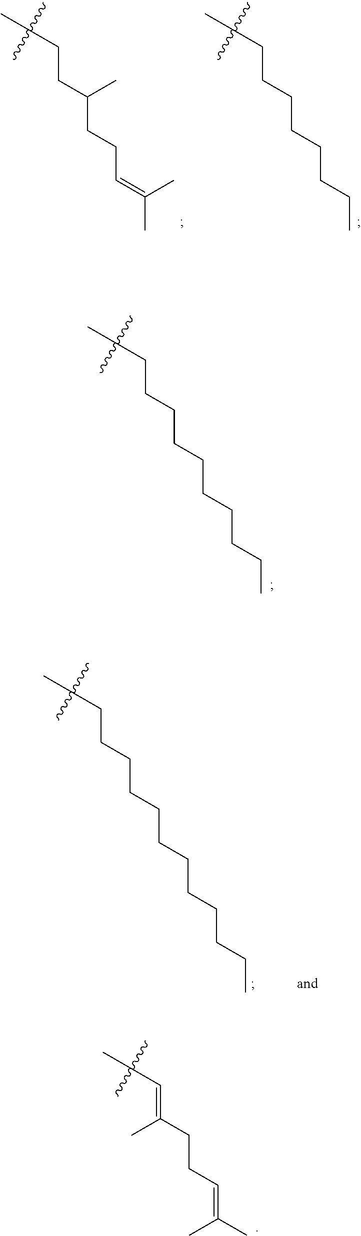

Alternatively, R⁴ may be selected from: H;

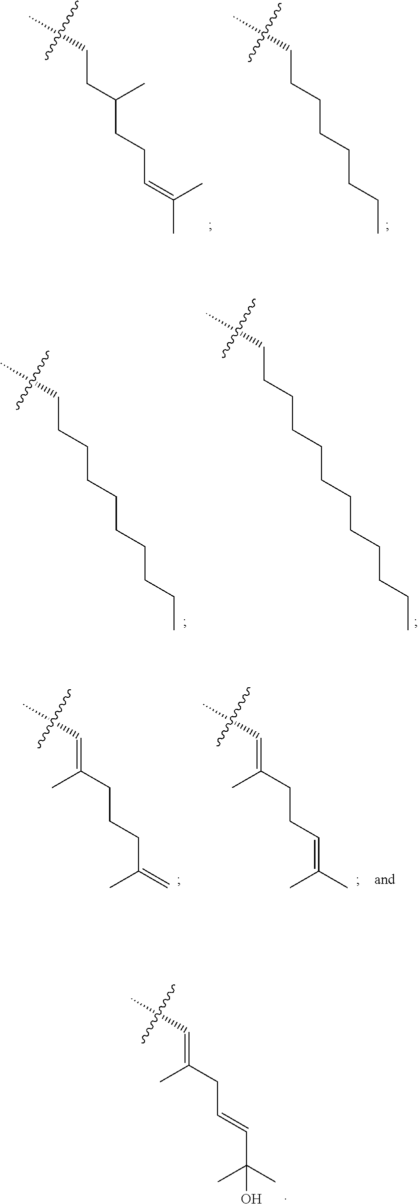

Alternatively, R⁴ may be selected from:

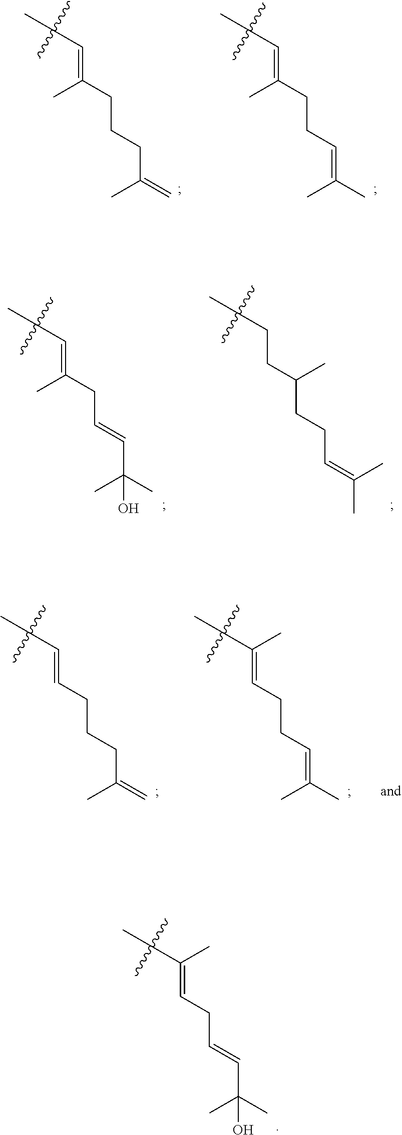

Alternatively, R⁴ may be selected from:

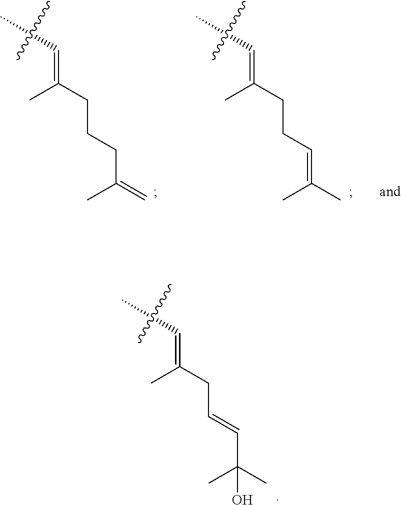

Alternatively, R⁴ may be selected from:

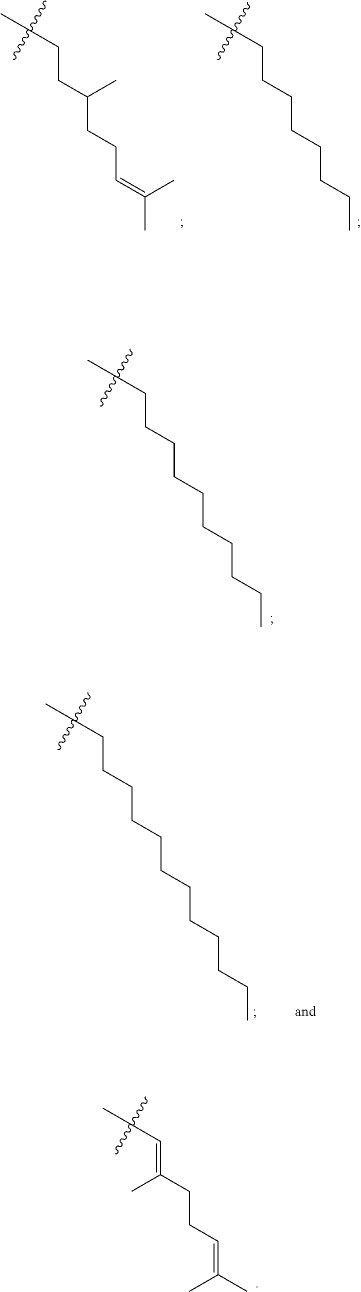

Alternatively, R⁴ may be selected from:

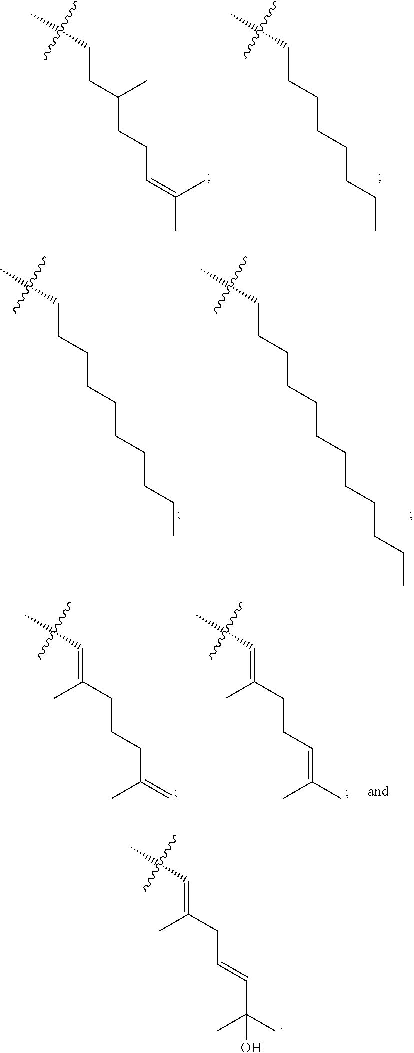

Alternatively, R⁴ may be selected from:

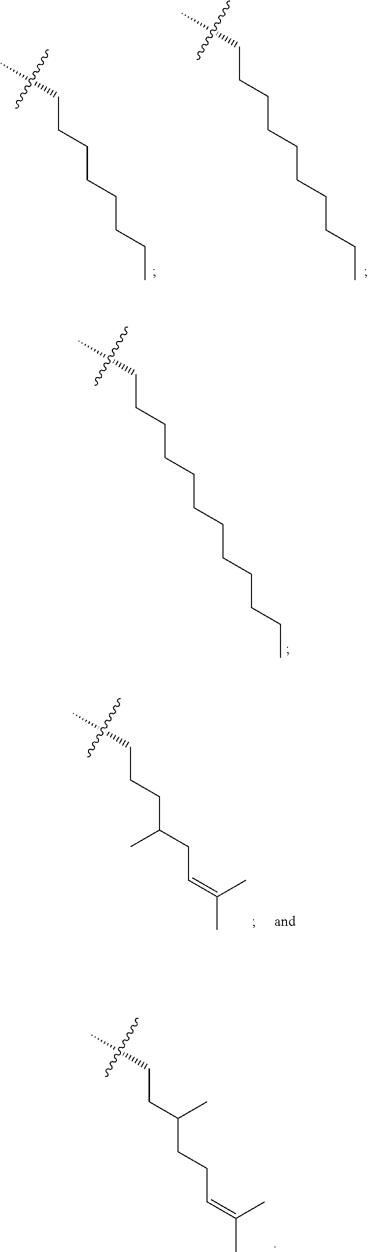

Alternatively, R² and R³ may be independently selected from: H; OC(═O)CH₃; OC(═O)CH₂CH(CH₃)CH₃; and OH; or jointly are ═CH₂. Alternatively, X¹ may be selected from: O; and S; and X² is O. Alternatively, R¹ may be H. The compound may be selected from one or more of the following:

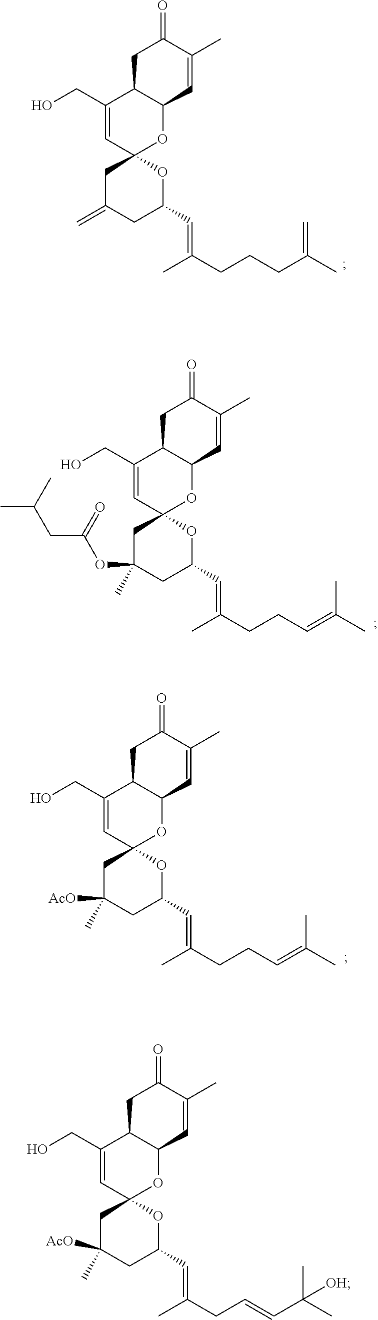

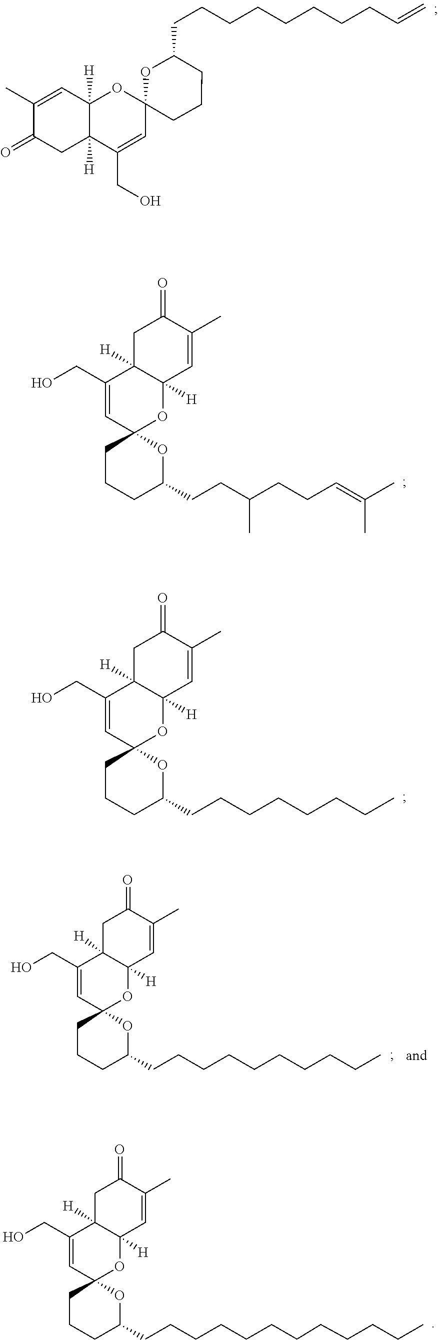 [SMILES: C=CCCCCCCCC[C@@H]1CCC[C@]2(C=C(CO)[C@H]3CC(=O)C(C)=C[C@H]3O2)O1 ; CC(C)=CCCC(C)CC[C@@H]1CCC[C@]2(C=C(CO)[C@H]3CC(=O)C(C)=C[C@H]3O2)O1 ; CCCCCCCC[C@@H]1CCC[C@]2(C=C(CO)[C@H]3CC(=O)C(C)=C[C@H]3O2)O1 ; CCCCCCCCCC[C@@H]1CCC[C@]2(C=C(CO)[C@H]3CC(=O)C(C)=C[C@H]3O2)O1 ; CCCCCCCCCCCC[C@@H]1CCC[C@]2(C=C(CO)[C@H]3CC(=O)C(C)=C[C@H]3O2)O1]

Alternatively, the compound may be selected from one or more of the following:

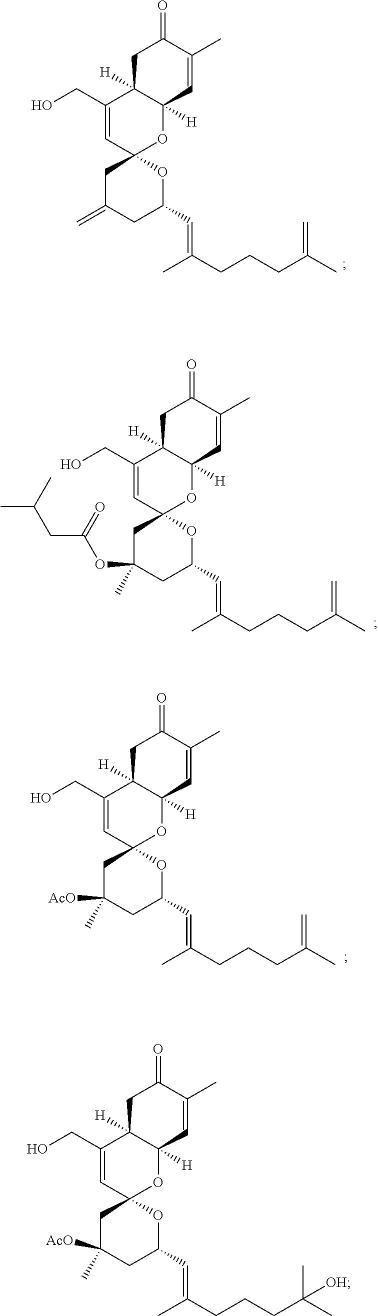

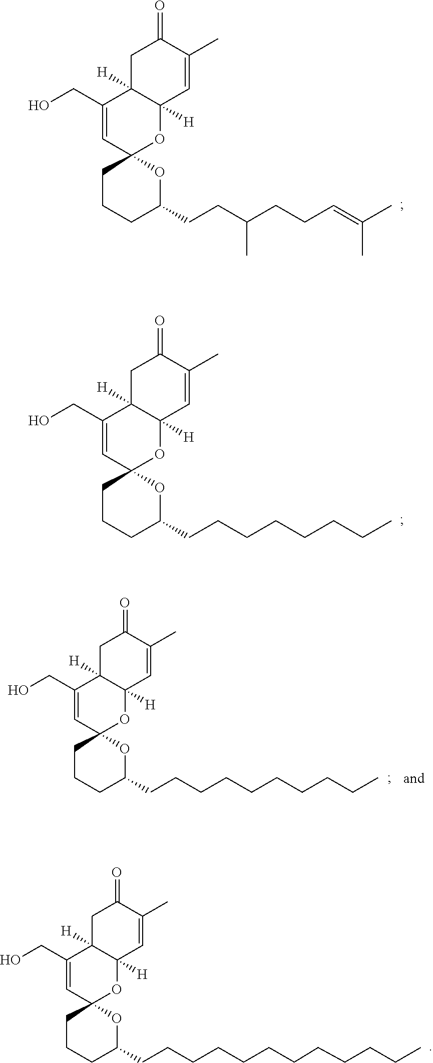 [SMILES: CC(C)=CCCC(C)CC[C@@H]1CCC[C@]2(C=C(CO)[C@H]3CC(=O)C(C)=C[C@H]3O2)O1 ; CCCCCCCC[C@@H]1CCC[C@]2(C=C(CO)[C@H]3CC(=O)C(C)=C[C@H]3O2)O1 ; CCCCCCCCCC[C@@H]1CCC[C@]2(C=C(CO)[C@H]3CC(=O)C(C)=C[C@H]3O2)O1 ; CCCCCCCCCCCC[C@@H]1CCC[C@]2(C=C(CO)[C@H]3CC(=O)C(C)=C[C@H]3O2)O1]

The compound may be selected from one or more of the following:

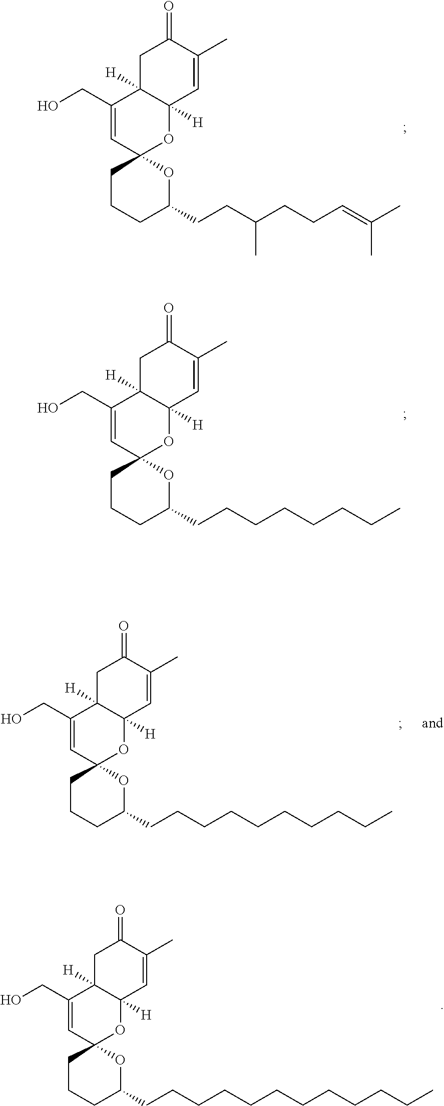 [SMILES: CC(C)=CCCC(C)CC[C@@H]1CCC[C@]2(C=C(CO)[C@H]3CC(=O)C(C)=C[C@H]3O2)O1 ; CCCCCCCC[C@@H]1CCC[C@]2(C=C(CO)[C@H]3CC(=O)C(C)=C[C@H]3O2)O1 ; CCCCCCCCCC[C@@H]1CCC[C@]2(C=C(CO)[C@H]3CC(=O)C(C)=C[C@H]3O2)O1 ; CCCCCCCCCCCC[C@@H]1CCC[C@]2(C=C(CO)[C@H]3CC(=O)C(C)=C[C@H]3O2)O1]

The compound may be selected from one or more of the following:

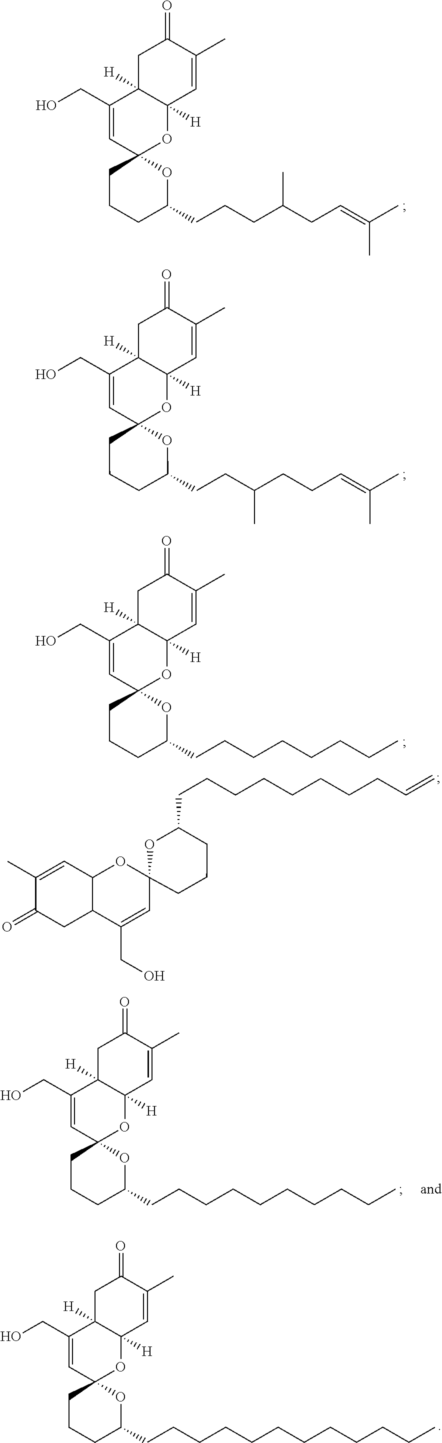

The compound may be selected from one or more of the following:

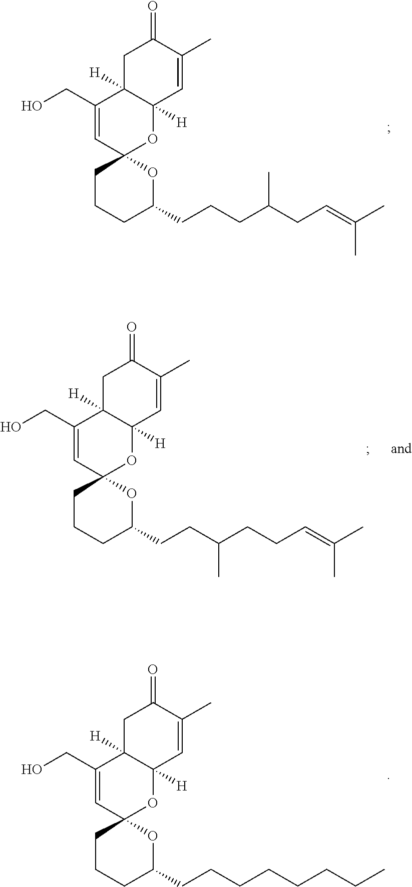 [SMILES: CC(C)=CCC(C)CCC[C@@H]1CCC[C@]2(C=C(CO)[C@H]3CC(=O)C(C)=C[C@H]3O2)O1 ; CC(C)=CCCC(C)CC[C@@H]1CCC[C@]2(C=C(CO)[C@H]3CC(=O)C(C)=C[C@H]3O2)O1 ; CCCCCCCC[C@@H]1CCC[C@]2(C=C(CO)[C@H]3CC(=O)C(C)=C[C@H]3O2)O1]

The coronavirus infection may be selected from one or more of the following: Severe Acute Respiratory Syndrome (SARS) coronavirus-1 (SARS-CoV-1) infection; SARS coronavirus-2 (SARS-COV-2) infection; and Middle East Respiratory Syndrome (MERS) coronavirus (MERS-CoV) infection. The coronavirus infection may be human coronavirus 229E (HCoV-229E) infection. The coronavirus infection may be from a HCoV-229E variant selected from: Alpha; Beta; Gamma; Delta; and Omicron. The coronavirus infection may be from a HCoV-229E variant selected from: Omicron and Delta. The coronavirus infection may be from a HCoV-229E variant selected from: Omicron BA.1; Omicron BA.2; Omicron BA.5; or Delta B.1.617.2.

Alternatively, R⁴ may be a one to twenty carbon saturated, unsaturated, linear, or branched alkyl group. Alternatively, R⁴ may be a one to twenty carbon saturated, unsaturated, linear, or branched alkyl group, where the individual carbon atoms may be substituted with O, OH, ═O, NH, SH, F, Cl, and Br. Alternatively, R⁴ may be a one to twenty carbon saturated, unsaturated, linear, or branched alkyl group, where the individual carbon atoms may be substituted with O, OH, NH, F, Cl, and Br.

Alternatively, R⁴ may be a one to twenty carbon saturated, unsaturated, linear, or branched alkyl group, where the individual carbon atoms may be substituted with OH.

Alternatively, R⁴ may be a one to twenty carbon saturated, unsaturated, linear, or branched alkyl group, where the individual carbon atoms are unsubstituted.

Alternatively, R² may be selected from: H; OC(═O)CH₃; and OC(═O)CH₂CH(CH₃)CH₃; and R₃ may be selected from: H; and CH₃. Alternatively, R² may be H and R₃ may be H.

Alternatively, compound may be selected from:

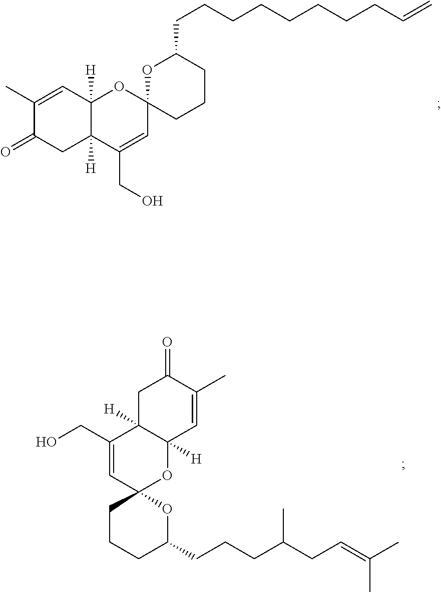 [SMILES: C=CCCCCCCCC[C@@H]1CCC[C@]2(C=C(CO)[C@H]3CC(=O)C(C)=C[C@H]3O2)O1 ; CC(C)=CCC(C)CCC[C@@H]1CCC[C@]2(C=C(CO)[C@H]3CC(=O)C(C)=C[C@H]3O2)O1]

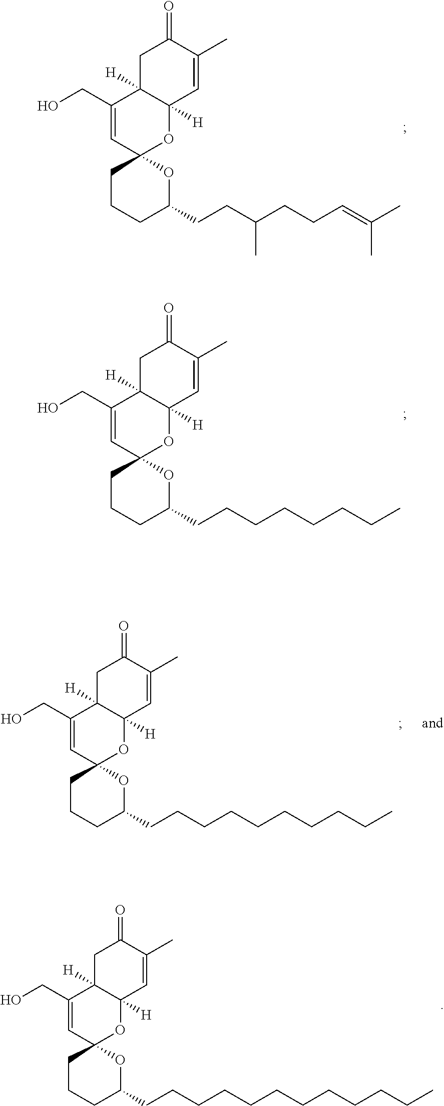 [SMILES: CC(C)=CCCC(C)CC[C@@H]1CCC[C@]2(C=C(CO)[C@H]3CC(=O)C(C)=C[C@H]3O2)O1 ; CCCCCCCC[C@@H]1CCC[C@]2(C=C(CO)[C@H]3CC(=O)C(C)=C[C@H]3O2)O1 ; CCCCCCCCCC[C@@H]1CCC[C@]2(C=C(CO)[C@H]3CC(=O)C(C)=C[C@H]3O2)O1 ; CCCCCCCCCCCC[C@@H]1CCC[C@]2(C=C(CO)[C@H]3CC(=O)C(C)=C[C@H]3O2)O1]

In a further aspect, there is provided a method of preventing, inhibiting, or reducing the viral activity of a coronavirus, such as SARS-Cov-2, on or in a cell or a subject which comprises administering to the cell or the subject an effective amount of a compound having the structural formula depicted in Formula 1,

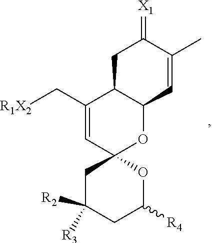

wherein: X₁ may be either O, S, N—OH, N—NH—R₅; X₂ may be either O or S; R₁ is H, or a one to ten carbon saturated, or unsaturated, linear, branched, or cyclic, alkyl group where individual carbon atoms may be substituted with OH, OR, ═O, NH, SH, F, Cl, Br, or be replaced by N, or S; R₂ and R₃ are independently H, R, OR, or R₂ and R₃ jointly form ═CR⁶R⁷; R₄ is H or a one to twenty carbon saturated, unsaturated, or aromatic, linear, branched, or cyclic alkyl group where individual carbon atoms may be substituted with O, OH, OR, ═O, NH, SH, F, Cl, Br, or be replaced by N, or S; R₅ may be a one to ten carbon saturated, or unsaturated, linear, branched, or cyclic, alkyl group where individual carbon atoms may be substituted with OH, OR, ═O, NH, SH, F, Cl, Br, or be replaced by N, or S; R⁶ may be H, a one to ten carbon saturated, or unsaturated, linear, branched, or cyclic, alkyl group where individual carbon atoms may be substituted with OH, OR, ═O, NH, SH, F, Cl, Br, or be replaced by N, or S; R₇ may be H, or a one to ten carbon saturated, or unsaturated, linear, branched, or cyclic, alkyl group where individual carbon atoms may be substituted with OH, OR, ═O, NH, SH, F, Cl, Br, or be replaced by N, or S.

Another aspect of the invention is provided a method of preventing, inhibiting, or reducing the viral activity of a coronavirus, such as SARS-Cov-2, on or in a cell or a subject which comprises administering to the cell or the subject an effective amount of a compound having the structural formula depicted in Formula 2.

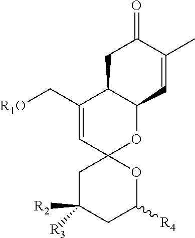

wherein: R₁ may be either H or CH₃C(O), wherein either R₂ is —OC(O)CH₃ and R₃ is CH₃; or, R₂ and R₃ jointly form ═CH₂; R₄ is H or a one to twenty carbon saturated, unsaturated, or aromatic, linear, branched, or cyclic alkyl group where individual carbon atoms may be substituted with O, OH, OR, ═O, NH, SH, F, Cl, Br, or be replaced by N, or S.

In yet another aspect of the invention, within the compounds of Formula 1 or Formula 2 the R₄ moieties may be H or a linear C8 to C12 alkyl chain, or one of the following:

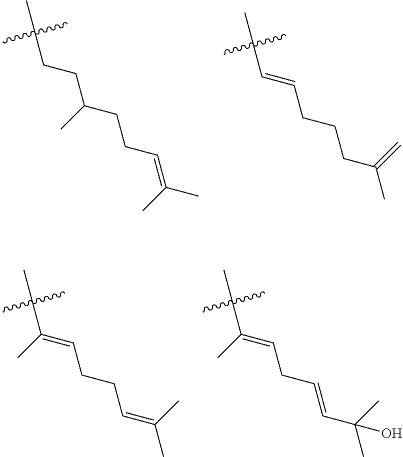

In another aspect of the invention is provided a method of preventing, inhibiting, or reducing the viral activity of a coronavirus, such as SARS-Cov-2, on or in a cell or a subject which comprises administering to the cell or the subject an effective amount of a compound selected from the following group:

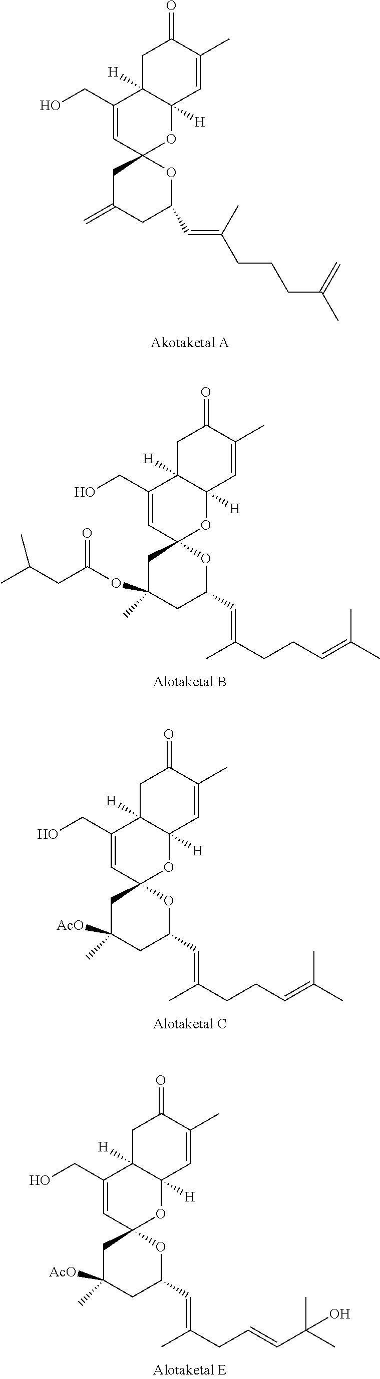

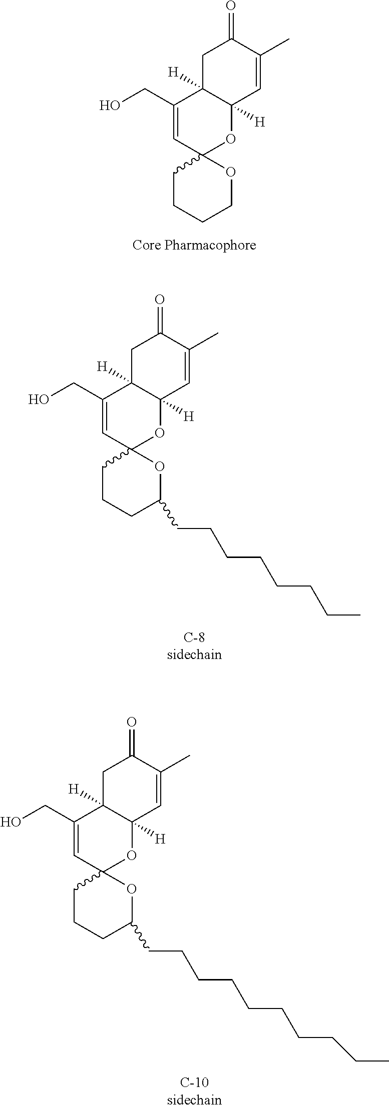 [SMILES: CC1=C[C@H]2OC3(C=C(CO)[C@H]2CC1=O)CCCCO3 ; CCCCCCCCC1CCCC2(C=C(CO)[C@H]3CC(=O)C(C)=C[C@H]3O2)O1 ; CCCCCCCCCCC1CCCC2(C=C(CO)[C@H]3CC(=O)C(C)=C[C@H]3O2)O1]

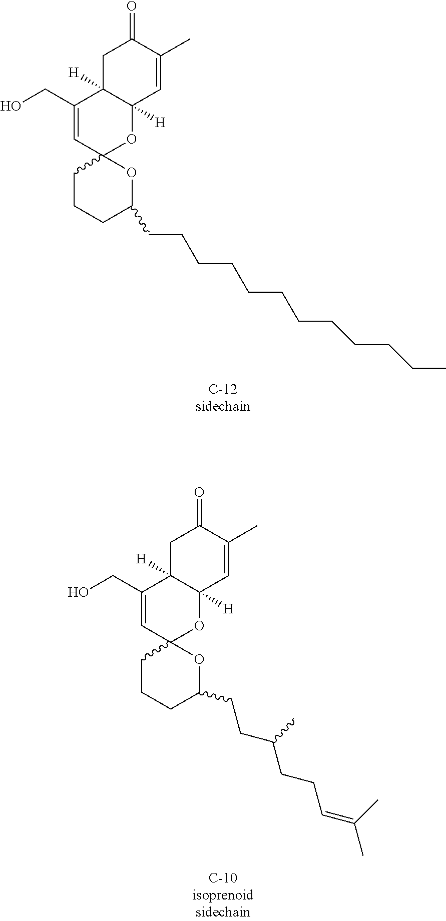 [SMILES: CCCCCCCCCCCCC1CCCC2(C=C(CO)[C@H]3CC(=O)C(C)=C[C@H]3O2)O1 ; CC(C)=CCCC(C)CCC1CCCC2(C=C(CO)[C@H]3CC(=O)C(C)=C[C@H]3O2)O1]

In one aspect of the invention, the coronavirus is SARS-CoV-2.

In a further aspect of the invention described herein are provided compounds of the following formulae:

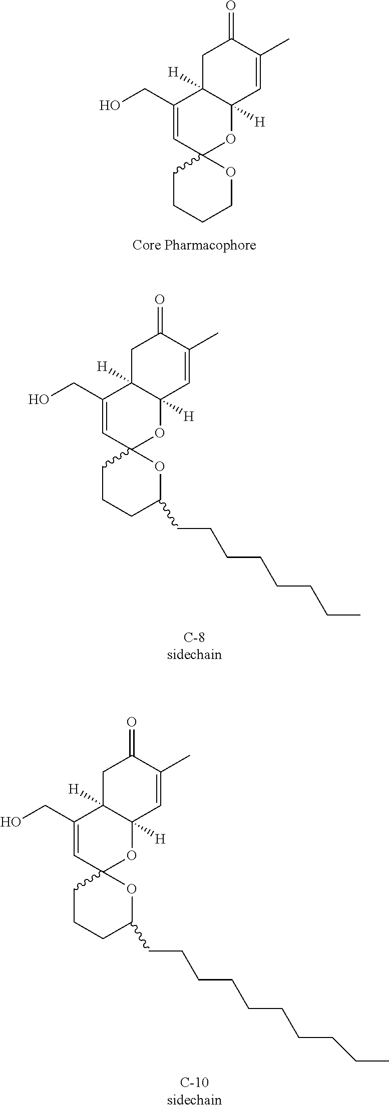 [SMILES: CC1=C[C@H]2OC3(C=C(CO)[C@H]2CC1=O)CCCCO3 ; CCCCCCCCC1CCCC2(C=C(CO)[C@H]3CC(=O)C(C)=C[C@H]3O2)O1 ; CCCCCCCCCCC1CCCC2(C=C(CO)[C@H]3CC(=O)C(C)=C[C@H]3O2)O1]

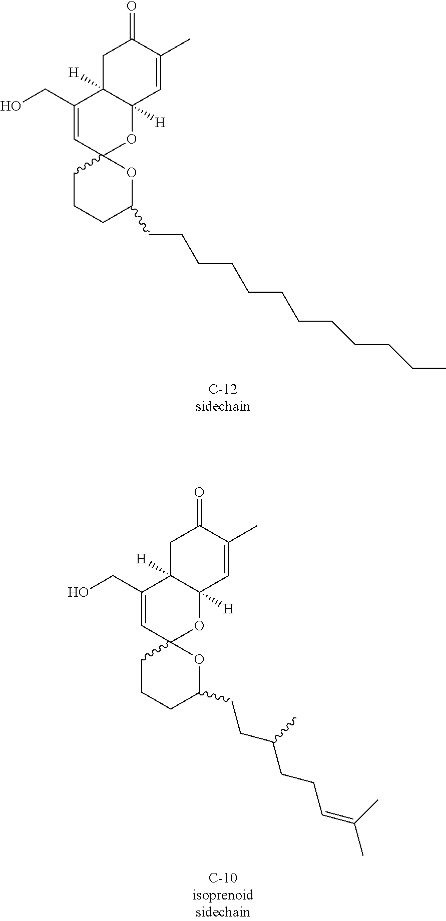 [SMILES: CCCCCCCCCCCCC1CCCC2(C=C(CO)[C@H]3CC(=O)C(C)=C[C@H]3O2)O1 ; CC(C)=CCCC(C)CCC1CCCC2(C=C(CO)[C@H]3CC(=O)C(C)=C[C@H]3O2)O1]

### BRIEF DESCRIPTION OF THE DRAWINGS

FIG. 1 shows dose-response curves from Calu-3 cells pretreated with the indicated concentrations of Alotaketal C before infection with SARS-CoV-2 VIDO, Delta (B.1.617.2), Omicron BA.1, Omicron BA.2 or Omicron BA-5, using nucleocapsid (circles) and dsRNA (squares) as infection markers, with EC₅₀ values determined using nonlinear regression analysis. The graph shows the average values of three independent experiments.

FIG. 2 shows dose-response curves were generated for positive control GC376 and Alotaketal C in Calu-3 cells infected with mNG-SARS-CoV-2 (n=3), where EC₅₀ values were determined using nonlinear regression analysis.

FIG. 3 shows Alotaketal C derivatives effect on SARS-CoV-2 Omicron Infection, with dose-response curves generated for Alotaketal C derivatives (I10-CIT3, I10-CIT4, I10-Dec01, I10-Dec02, I10-DD2, I10-H and I10-Oct) in Calu-3 cells infected with SARS-CoV-2 Omicron BA.1 using dsRNA (square) and nucleocapsid (circle) as infection markers (n=3). The GraphPad Prism 9™ (GraphPad Software, Inc.) nonlinear regression fit modeling variable slope was used to generate a dose-response curve [Y=Bottom+(Top-Bottom)/(1+10\hat{ }((Log IC₅₀−X)*HillSlope)], constrained to top=100, bottom=0.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown. Any terms not directly defined herein shall be understood to have the meanings commonly associated with them as understood within the art of the invention.

To address the need for anti-SARS-CoV-2 therapy, a cell-based high-content screen (HCS) assay using mNeoGreen reporter SARS-CoV-2 virus (Xie 2020), was performed with a library of 405 natural products. Positive candidate natural products were found able to inhibit SARS-CoV2 infection in Calu-3 cells, with half-maximal effective dose (ED₅₀) values ranging between 1 and 50 μM, and showed more than 80% cell viability. In particular, Alotaketal C (Daoust J 2013), shows antiviral activity in the nanomolar range with broad-spectrum activity by inhibiting VOCs infection.

Emerging viruses like SARS-CoV-2 can cause global pandemics with the potential for serious health problems. Even if the vaccination campaigns are ongoing worldwide, there is still a tremendous need for the development of new therapeutics that are safe and easily distributable to a wide range of populations. These require further research into promising antiviral candidates to develop efficient solutions against SARS-CoV-2 infections.

Natural products are considered a rich resource for novel antiviral drug development. Apart from plant-derived compounds, like Nigella sativa with inhibitory activity against the hepatitis C virus, several marine products (Wang 2014, Wang M 2016) are also reported for their antiviral capacities against different viruses. Some natural products have been found through the inhibition of viral replication (Moghadamtousi 2015, Oliveira 2017). The antiviral activity can be performed based on their capability to inhibit viral entry, viral DNA and RNA synthesis, as well as viral reproduction, but they can also modulate cellular biological functions offering a broad-spectrum antiviral activity (Mussarra-Pizzo 2021). They have the advantages of a better toxicological profile, fewer side effects and a faster admission process in comparison to chemically engineering drugs (Auth 2021). Some NP has proved promising against other coronaviruses, like SARS-CoV and MERS-CoV (Mani 2020, Ashhurst 2021).

We tested a library of natural products, of large diversity, for their ability to inhibit infection of SARS-CoV-2 and VOCs. Even if SARS-CoV-2 can infect different cells, we reasoned that using Calu-3 cells for our cell-based screening would give us more accurate results, since these cells are highly permissive to SARS-CoV-2, with a more robust replication (Chu 2020). Our high-content screening assay designed to use mNG-SARS-CoV-2 as a reporter allows us to identify compounds able to inhibit the virus infection. The criteria for the inhibition rate associated with signals in antiviral HCS assays range between 60 to 90% according to different references (Shen 2019, Guo 2020, Lee 2018, Cox 2018), we focused on screening hits with higher potency. Therefore, we establish the inhibition threshold at 80% with a cell lose value at 20%. Finally, we identified twenty-five compounds exhibiting anti-SARS-CoV-2 activity. The Z′ values of each screening were between 0.5 and 0.7, which has an average value of 0.6, which confirmed the reliability of our assays. Among the twenty-five NP prime candidates, fifteen compounds showed inhibitory effects on virus infection in a dose-dependent manner with an EC₅₀<10 μM and SI index>10, some of these compounds were described by presenting, anti-inflammatory (Wu 2019), anti-malaria (Khumkomhet 2009, Chaiyosang 2016) or anti-cancer activities (Bao 2018).

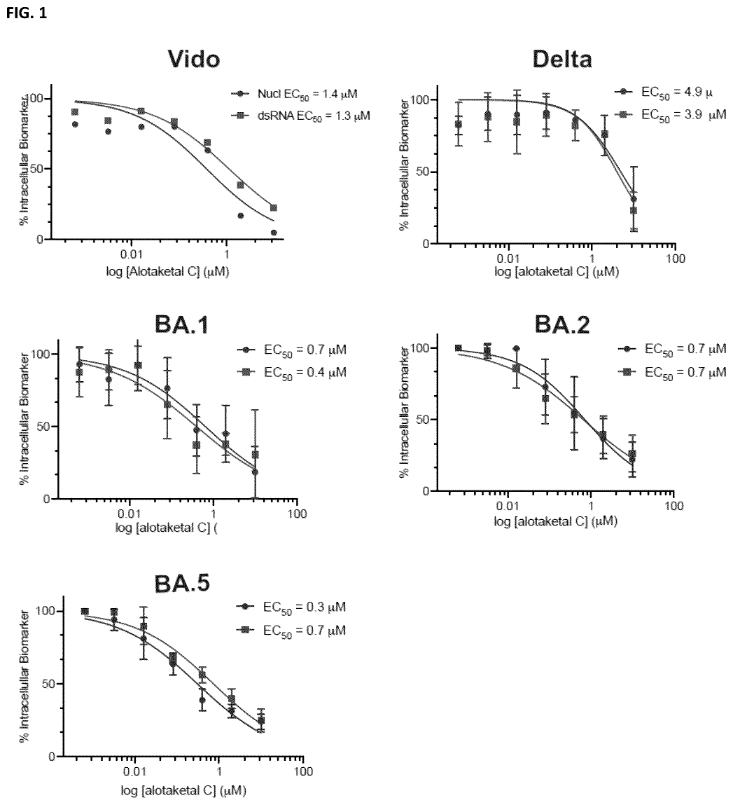

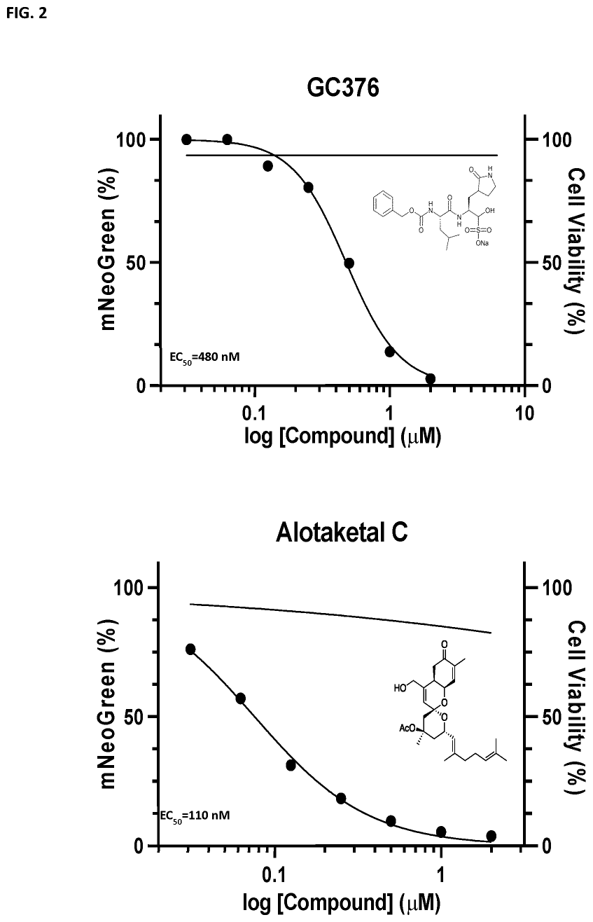

Alotaketal C showed potent inhibitory effects on mNG-SARS-CoV-2 infection in a dose-dependent manner with nanomolar activity, with an EC₅₀ value of 0.11 μM. The usefulness of these two compounds needs to be envisaged in the context of the critical emergence of VOCs in the SARS-CoV-2 pandemic. The potential antiviral activity Alotaketal C was tested by dose-dependent reduction of SARS-CoV-2 VIDO, Delta and Omicron infection and validated using two viral biomarkers of intracellular infection (dsRNA and nucleocapsid). The EC₅₀ value determined for the dsRNA and the nucleocapsid can be seen in FIG. 1. However, it does not show the nanomolar activity determined with mNG-SARS-CoV-2 (FIG. 2; Pérez-Vargas 2023), this could be due to the spheroid-like phenotype presented in cells infected with VOCs, more pronounced in Delta infection, (FIG. 1); making the EC₅₀ values less accurate given the quantitative limitation associated with spheroid-like cells (Leary 2018). Spheroid-like cells have multiple cell layers that create gradients and barriers. To assess activities as a function of radial position within a spheroid-like cell, the images must be high magnification to get accurate quantitative depictions (Leary 2018). However, this phenomenon allows us to observe the effect of NPs inhibiting virus infection by reversing the spheroid-like morphology and recovering the mock non-infected morphology (Shapira 2022). Nevertheless, our results confirmed the broad-spectrum antiviral activity of Alotaketal C against SARS-CoV-2 variants.

Alotaketal C is a sesterterpenoid isolated from the marine sponge Phorbas sp. collected in Canada, it was shown to pose a stronger response than prostratin and activates proteins Kinase C (PKC), acting as provirus expression for latent HIV-1 (Wang 2016). Prostratin can activate latent virus reservoirs while preventing healthy cells from infection (Beans 2013). The immune evasion mechanism of coronavirus is due to the inhibition of Type-I interferon (IFN) production. Lack of type I IFN leads to defects in antibody production, effector T-cell response, expression of INF-stimulating genes and decreased antigen presentation. Activation of PKC is involved in the generation of IFN antiviral activity response (Zorzitto 2006, Vabret 2020, Lei 2020). However, we do not have any evidence that suggests this mode of action of Alotaketal C against SARS-CoV-2.

It will be understood by a person of skill that NR₂ may include the corresponding ion, for example, ammonium ions. Alternatively, where the ions are shown, a person of skill in the art will appreciate that the counter ion may also be present.

Those skilled in the art will appreciate that the point of covalent attachment of the moiety to the compounds, as described herein, may be, for example, and without limitation, cleaved under specified conditions. Specified conditions may include, for example, and without limitation, in vivo enzymatic or non-enzymatic means. Cleavage of the moiety may occur, for example, and without limitation, spontaneously, or it may be catalyzed, induced by another agent, or a change in a physical parameter or environmental parameter, for example, an enzyme, light, acid, temperature or pH. The moiety may be, for example, and without limitation, a protecting group that acts to mask a functional group, a group that acts as a substrate for one or more active or passive transport mechanisms, or a group that acts to impart or enhance a property of the compound, for example, solubility, bioavailability or localization.

In some embodiments, compounds, as described herein, may be used for the treatment of viral infection. Alternatively, the compounds, described herein, may be used to interfere with viral RNA processing including splicing, polyadenylation, and export to the cytoplasm. Alternatively, the compounds that may be suitable for the treatment of Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection. The viral infection may be the viral infection is human coronavirus 229E (HCoV-229E) infection.

Compounds, as described herein, may be in the free form or in the form of a salt thereof. In some embodiment, compounds, as described herein, may be in the form of a pharmaceutically acceptable salts, which are known in the art. Pharmaceutically acceptable salt, as used herein, include, for example, salts that have the desired pharmacological activity of the parent compound (salts which retain the biological effectiveness and/or properties of the parent compound and which are not biologically and/or otherwise undesirable). Compounds, as described herein, having one or more functional groups capable of forming a salt, may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid. Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, i-ephenamine, N,N′-dibenzylethylenediamine or polyamine resins. In some embodiments, compounds as described herein may contain both acidic and basic groups and may be in the form of inner salts or zwitterions, for example, and without limitation, betaines. Salts as described herein may be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

In some embodiments, compounds and all different forms thereof (e.g. free forms, salts, polymorphs, hydrates, hydrated salts, optical isomers, racemates, diastereoisomers, enantiomers, isomeric forms) as described herein may be in a solvent addition form, for example, solvates. Solvates contain either stoichiometric or non-stoichiometric amounts of a solvent in physical association with the compound or salt thereof. The solvent may be, for example, and without limitation, a pharmaceutically acceptable solvent. For example, hydrates are formed when the solvent is water and alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudo-polymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formulas illustrated for the sake of convenience.

In some embodiments, pharmaceutical compositions as described herein may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents (used interchangeably herein) are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein micro-particles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20th ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene polyoxypropylene copolymers may be used to control the release of the compounds. Other potentially useful parenteral delivery systems for modulatory compounds include ethylene vinyl acetate copolymer particles, osmotic pumps, implantable infusion systems, and liposomes. Formulations for inhalation may contain excipients, for example, lactose, or may be aqueous solutions containing, for example, polyoxyethylene 9 lauryl ether, glycocholate and deoxycholate, or may be oily solutions for administration in the form of nasal drops, or as a gel.

Compounds or pharmaceutical compositions as described herein or for use as described herein may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition as described herein includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to an androgen independent form. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds, as described herein, should be used without causing substantial toxicity. Toxicity of the compounds as described herein can be determined using standard techniques, for example, by testing in cell cultures or experimental animals and determining the therapeutic index, i.e., the ratio between the LD₅₀ (the dose lethal to 50% of the population) and the LD₁₀₀ (the dose lethal to 100% of the population). In some circumstances however, such as in severe disease conditions, it may be appropriate to administer substantial excesses of the compositions. Some compounds as described herein may be toxic at some concentrations. Titration studies may be used to determine toxic and non-toxic concentrations. Animal studies may be used to provide an indication if the compound has any effects on other tissues.

Compounds, as described herein, may be administered to a subject. As used herein, a “subject” may be a human, non-human primate, rat, mouse, cow, horse, pig, sheep, goat, dog, cat, etc. The subject may be suspected of having or at risk for having a viral infection, such as SARS-CoV-1, SARS-CoV-2, and MERS-CoV. Alternatively, the infection may be a coronavirus infection. Diagnostic methods for various viral infections, are known to those of ordinary skill in the art.

As used herein, the term “Coronavirus” abbreviated CoV refers to a family of enveloped, positive-sense, single-stranded, and highly diverse RNA viruses, with four distinct groups (i.e. alpha, beta, gamma, and delta). The α-coronavirus and β-coronavirus are of particular interest, because of their ability to cross from non-human animals to humans. So far, there are seven documented human coronaviruses (hCoVs), including the beta-genera CoVs, namely Severe Acute Respiratory Syndrome (SARS)-CoV (SARS-CoV), Middle East Respiratory Syndrome (MERS)-CoV (MERS-CoV), SARS-CoV hCoV-HKU1, and hCoV-OC43 and the α-genera CoVs, which are hCoV-NL63 and hCoV-229E, respectively.

Any terms not directly defined herein shall be understood to have the meanings commonly associated with them as understood within the art of the invention.

Various alternative embodiments and examples are described herein. These embodiments and examples are illustrative and should not be construed as limiting the scope of the invention.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Various alternative embodiments and examples are described herein. These embodiments and examples are illustrative and should not be construed as limiting the scope of the invention.

### Materials and Methods

### Cell Lines and Antibodies

Calu-3 cells (ATCC™ HTB-55™) were cultivated according to ATCC recommendations. The SARS-CoV-2 nucleocapsid antibody [HL344](GTX635679) was kindly provided by Genetex™; mouse anti-dsRNA antibody (J2) was purchased from Scicons English and Scientific Consulting (10010500); secondary antibodies goat anti-mouse IgG Alexa Fluor 488™ (A11001) and goat anti-rabbit IgG Alexa Fluor 555™ (A21428) were obtained from Thermo Fisher Scientific™.

### SARS-CoV-2 Viruses

All infections were carried out in a Biosafety Level 3 (BSL3) facility (UBC FINDER) in accordance with the Public Health Agency of Canada and UBC FINDER regulations (UBC BSL3 Permit #B20-0105 to FJ). SARS-CoV-2 (SARS-CoV-2/Canada/VIDO-01/2020) was kindly provided by Dr. Samira Mubareka (Sunnybrook, ON, Canada). SARS-CoV-2 VOC (B.1.617.2; Delta and BA.1; Omicron) was kindly provided by Dr. Mel Krajden (BC Centre for Disease Control, BC, Canada). SARS-CoV-2 Omicron BA.5 was isolated by Dr. Masahiro Niikura (SFU) from a clinical specimen. Viral stocks were made in Vero E6 cells (Ogando, N. S. 2020).

### Natural Products

The natural product screening library contained 405 pure natural products representing a large diversity of chemical scaffolds (See Supporting Information for all the chemical structures) isolated from a broad spectrum of plants, invertebrates, or microorganisms collected in diverse terrestrial and marine habitats. Terrestrial plants and microorganisms were collected in Thailand, Brazil, Canada, and Sri Lanka. Marine invertebrates and microorganisms were collected in ocean waters off the coasts of Canada, Brazil, Italy, Papua New Guinea, Indonesia, Dominica, and Sri Lanka. Most natural products in the screening library were discovered and first reported in the literature by the co-authors. Some of the pure natural products isolated by the co-authors had been previously reported in the literature by other research groups. The structures of all the natural products discovered by the co-authors were elucidated by detailed analysis of nuclear magnetic resonance (NMR) spectroscopy, mass spectrometry (MS), and/or single-crystal X-ray diffraction data. The structures of known compounds were confirmed by comparing their NMR and MS data to literature values. The purity of compounds was determined by HPLC and NMR analyses. Literature references detailing the discovery of the significantly active compounds are provided.

### SARS-CoV-2 Infections and Fluorescent Staining

Calu-3 cells were seeded at a concentration of 10⁴ cells/well in 96-well plates the day before infection. Cells were pretreated for 3 hr. with fixed (50 μM for screening) or diluted concentrations of compounds (indicated in figure legends) followed by infection with SARS-CoV-2 VOCs at a multiplicity of infection (MOI) of 1 for 2 days, followed by fixation of the cells with 3.7% formalin for 30 min to inactivate the virus. The fixative was removed, and the cells were washed with PBS, permeabilized with 0.1% Triton X-100 for 5 min and blocked with 1% bovine serum albumin (BSA) for 1 hr, followed by immunostaining with rabbit primary antibody HL344 (SARS-CoV-2 nucleocapsid) and/or mouse primary antibody J2 (dsRNA) at working dilutions of 1:1000 for 1 hr at room temperature. Secondary antibodies were used at a 1:2000 dilution and Hoechst™ was used at 1 μg/mL for 1 hr at room temperature in the dark. After washing with PBS, the plates were kept in the dark at 4° C. until imaging on a high-content screening (HCS) platform (CellInsight CX7 HCS™, Thermo Fisher Scientific™) with a 10× objective.

### High-Content Screening

Monitoring of the total number of cells (based on nuclei staining) and the number of virus-infected cells (based on mNG expression) was performed using the CellInsight CX7 HCS™ platform (Thermo Fisher Scientific™), as previously described (Shapira 2022; Olmstead 2012). Briefly, nuclei are identified and counted using 350/461 nm wavelength (Hoechst 33342™); cell debris and other particles are removed based on a size filter tool. A region of interest (ROI, or “circle”) is then drawn around each host cell and validated against the bright field image to correspond with host cell membranes. The ROI encompasses the “spots” where mNG (485/521 nm wavelength) or another viral marker (dsRNA (485/521 nm wavelength) and nucleocapsid (549/600 nm wavelength) are localized. Finally, the software (HCS Studio Cell Analysis Software™, version 4.0) identifies, counts, and measures the pixel area and intensity of the “spots” within the “circle”. The fluorescence measured within each cell (circle) is then added and quantified for each well. The total circle-spot intensity of each well corresponds to intracellular virus levels (Z′>0.6) and is normalized to non-infected cells and infected cells treated with 0.1% DMSO. Nine fields were sampled from each well. NP hits were selected by higher inhibition effect (80%) and low cell loss (20%).

### Half Maximal Effective Dose (EC₅₀) Curves

Intracellular dose response (EC₅₀ values) for selected compounds against mNG SARS-CoV-2 were determined by pre-treating Calu-3 cells for 3 hr with serially diluted compounds (0.64, 3.2, 16, 80, 400, 2000, and 10000 nM), followed by virus infection for 2 days. Viral infection was detected by mNG fluorescence. EC₅₀ experiments were repeated at least three times. Intracellular fluorescent levels were interpolated to negative control (0.1% DMSO, no infection)=0, and positive control (0.1% DMSO, with infection)=100. The GraphPad Prism 9™ (GraphPad Software, Inc.™) nonlinear regression fit modeling variable slope was used to generate a dose-response curve [Y=Bottom+(Top-Bottom)/(1+10\hat{ }((Log IC50−X)*HillSlope)], constrained to top=100, bottom=0.

### Cytotoxicity Assays

Calu-3 cells were seeded with 104 cells/well in 96-well plates. Twenty-four hr after seeding media was aspirated and serially diluted compounds (described above) were added for an additional 48-hr. incubation. Cellular viability was assessed with Presto Blue Cell Viability Assay™ (Thermo Fisher Scientific™) according to the manufacturer's instructions. Cells were incubated with 5% PrestoBlue™ reagent for 2 hr before reading on the SpectraMax Gemini™ XS spectrofluorometer (Molecular Devices, LLC.™) set at excitation and emission wavelengths of 555 and 585 nm, respectively. Cellular viability was expressed relative (%) to vehicle-treated cells. Data are from at least three independent experiments.

### EXAMPLES

### Example 1: Validation of mNeoGreen-SARS-CoV-2

The mNeoGreen™ (mNG) reporter SARS-CoV-2 virus is going to be used for the high-content screening (HCS) assay, it was shown that the mNG reporter did not affect virus replication and remains stable (Xie 2020). Nevertheless, to validate our system, we compare the level of expression of mNG versus the nucleocapsid protein on Huh 7.5.1 cells (permissive cell line to SARS-CoV-2 infection) (Cagno 2020, Chu 2020) infected with mNG-SARS-CoV-2 in the presence of two broad-spectrum antivirals, Remdesivir™ and GC376. We can confirm the colocalization of mNG and the viral nucleocapsid in the same cells infected by mNG-SARS-CoV-2 (Pérez-Vargas 2023). Huh7.5.1 cells were pretreated with serial diluted Remdesivir™ or GC376 for 3 hours before mNG-SARS-CoV-2 infection, and we observed a dose-dependent reduction of mNG-SARS-CoV-2 infection in presence of both antivirals. The relative quantification of infected cells was used to determine EC₅₀ values (Half maximal effective concentration, drug concentration required to reduce infection by 50%) for Remdesivir™ and GC376, which were determined to be consistent for mNG and nucleocapsid (Pérez-Vargas 2023). We also tested GC376 in Calu-3 cells and we observed consistent EC₅₀ values (FIG. 2). These results demonstrated that mNG-SARS-CoV-2 is reliable and accurate for screening antiviral products in Huh7.5.1 and Calu-3 cells.

### Example 2: Screening of Natural Products

We aimed to identify natural products that can reduce SARS-CoV-2 infection, but also create and establish cell-based HCS that combines automated imaging and quantitative data analysis in a high-throughput format, which can be used with potential therapeutic libraries and different pathogens. In this assay, detection of mNG is used as a readout for viral infection and replication, and compound cytotoxicity was readout as cell loss. We look for compounds with antiviral activity capable of reducing mNG signal, without affecting cell viability. Optimal screening conditions were established using the mNG-SARS-CoV-2 reporter virus to infect Calu-3 cells with a density of 104 cells per 96-well plate, a multiplicity of infection (MOI) of 1, with pre-treatment of the cells with NPs for 3 hr and an endpoint of 48 hr post-infection. After the treatment cells were fixed and images are processed for quantitative analysis to evaluate the activity of the compound. HCS was performed with a library of 405 NP at a concentration of 50 μM, compounds showing inhibition of 80% of SARS-CoV-2 infection, with less than 20% of cell loss were defined as prime candidates (not shown); based on this criterion, twenty-five compounds were selected.

### Example 3: Dose-Response Curves of Hits

To confirm and prioritize the most promising compounds according to their anti-SARS-CoV-2 activity, the twenty-five compounds selected from the primary screening were tested for secondary screening with a broader concentration range (10-0.00064 μM) in Calu-3 cells. The relative quantification of infected cells was used to determine EC₅₀ values for the prime candidates, the values ranging from 0.08 to 45.60 μM (not shown). The half-maximal cytotoxic concentration (CC₅₀) values of these compounds were also calculated, no cytotoxicity was detected for either compound in the range of concentrations tested. Furthermore, the selective index (SI), expressed as the ratio of CC₅₀ on EC₅₀, indicates the potential as a new antiviral against SARS-CoV-2 infection, compounds. Alotaketal C showed 90.56% inhibition, an EC₅₀ of 0.08, an R² 0.79 and an SI value of 850.00.

### Example 4: Dose-Response Curves for Two NP Broad-Spectrum Inhibitors of SARS-CoV-2 Variants of Concern (VOCs)

Alotaketal C presents nanomolar activity (EC₅₀=0.11 μM) in the inhibition of mNG-SARS-CoV-2 infection (FIG. 2). VOCs possess a demonstrated capacity for enhanced transmission, disease severity, and reduced vaccine effectiveness (Hacisuleyman 2021, Plante 2021, Hoffmann 2021). SARS-CoV-2 B.1.617.2 (Delta), is presently the most prevalent VOC spreading worldwide and is leading to rapid increases in infection, we tested the impact of Alotaketal C, on Calu-3 cells pretreated with serially diluted compound for 3 hr before SARS-CoV-2 VIDO, Delta and Omicron infection. Using fluorescence high-content imaging we observed that Alotaketal C presents a dose-dependent reduction of SARS-CoV-2 VIDO, Delta and Omicron infection, demonstrated by a nucleocapsid and dsRNA staining (not shown) and with a reversal of the spheroid-like phenotype present in infected cells. Relative quantification of virally infected cells was used to determine EC₅₀ values (drug concentration required to reduce infection by 50%) for Alotaketal C, which were determined to be consistent for dsRNA (VIDO 1.33 μM; Delta: 3.96 μM; Omicron 1.54 μM) and nucleocapsid (VIDO 1.44 μM; Delta: 4.89 μM; Omicron: 0.14 μM) (FIG. 1). Together, these results underline the potential of Alotaketal C broad-spectrum inhibitors to act against emerging SARS-CoV-2 VOCs. Alotaketal C was also tested against SARS-CoV-2 Variants Delta B.1.617.2, Omicron BA.1, Omicron BA.2 and Omicron BA.5 as shown in FIG. 1 and TABLE 1.

TABLE 1
SARS-CoV-2 Variant Inhibition
(EC₅₀) by Alotaketal C in Calu3 Cells

| SARS-CoV-2 Variant | Nucleocapsid (EC₅₀) | dsRNA (EC₅₀) |
|---|---|---|
| Delta B.1.617.2 | 4.9 μM | 3.9 μM |
| Omicron BA.1 | 0.7 μM | 0.4 μM |
| Omicron BA.2 | 0.7 μM | 0.7 μM |
| Omicron BA.5 | 0.3 μM | 0.7 μM |

### Example 5: Synthetic Procedures

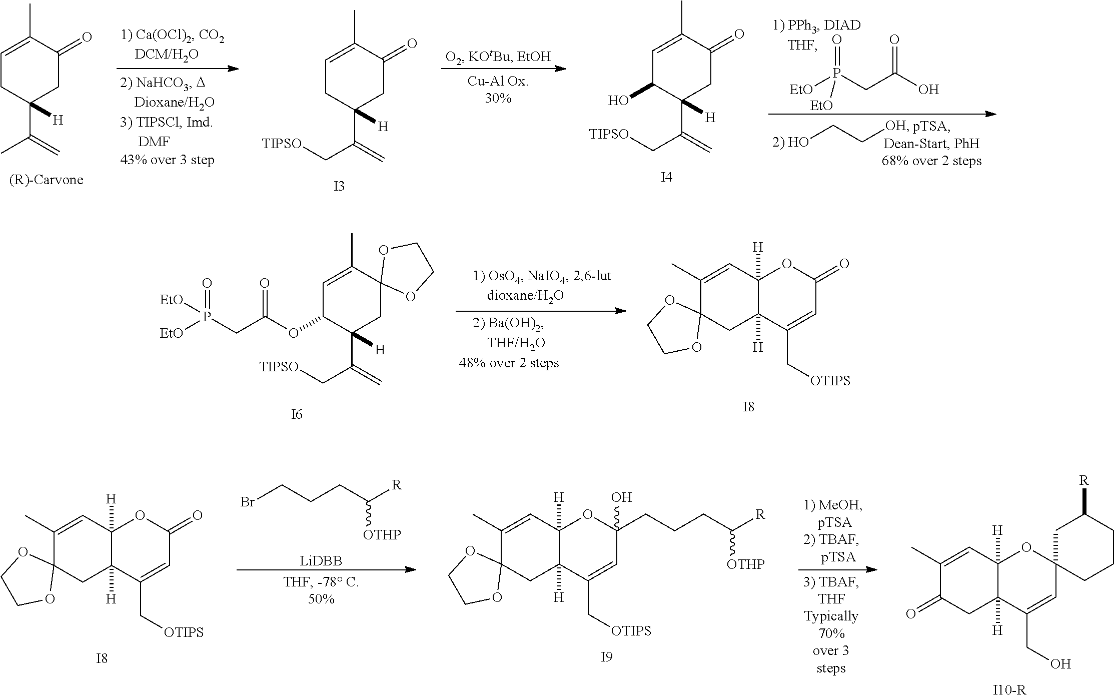

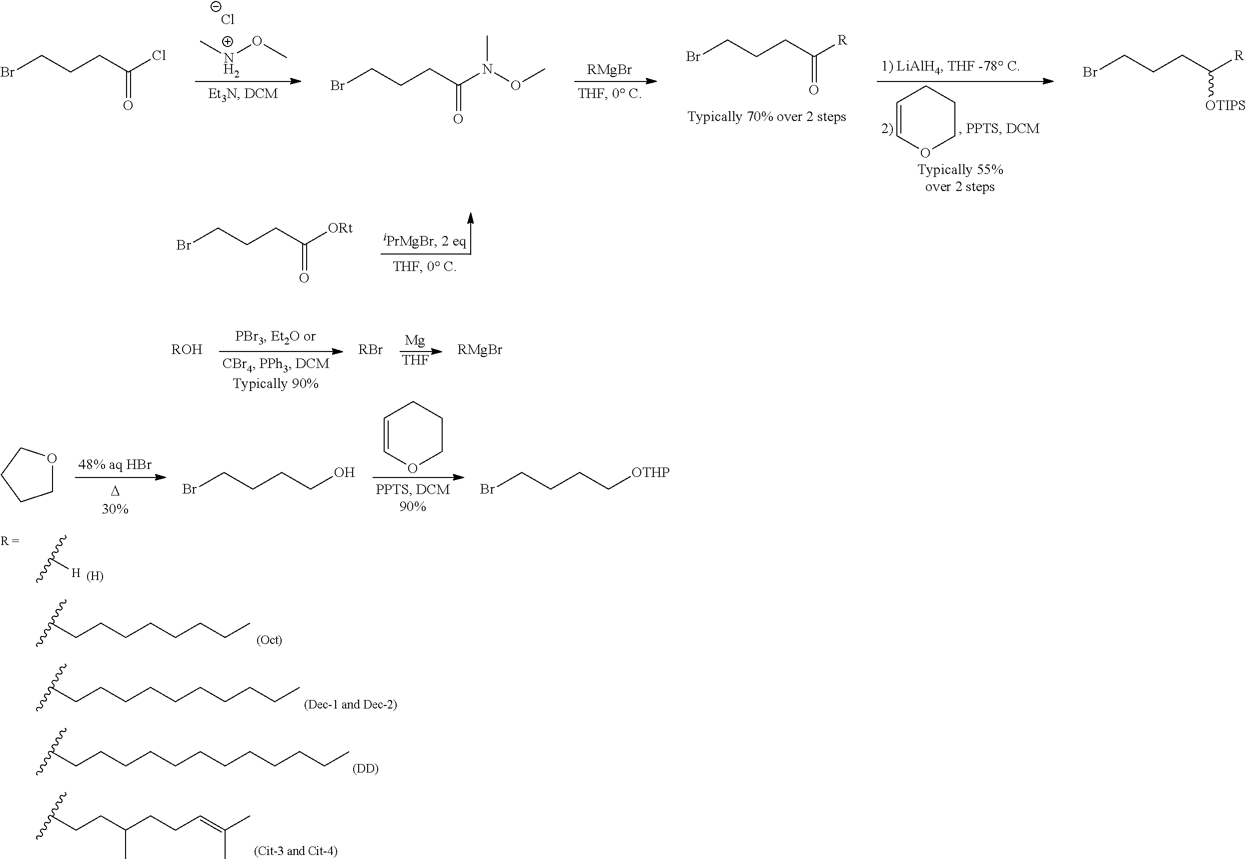

All chemicals and solvents (HPLC grade, reagent grade and/or anhydrous (from Sure-Seal™ bottles)) were purchased from Sigma Aldrich™ and used directly except where explicitly stated otherwise: acetonitrile (MeCN), p-anisaldehyde, benzene (PhH), calcium hypochlorite, (R)-carvone, diethyl ether (anhydrous; Et₂O), dichloromethane (DCM), 2-(diethoxyphosphoryl) acetic acid, dimethyl formamide (DMF), diisopropyl azodicarboxylate (DIAD), 1,4-dioxane, ethanol (absolute), ethyl acetate (EtOAC), ethylene glycol, imidazole, magnesium sulfate, methanol, mixture of hexanes (Hex), potassium tert-butoxide (KOtBu), potassium permanganate, phosphomolybdic acid, pyridine, pyridinium p-toluenesulfonate (PPTS), p-toluenesulfonic acid (pTSA) C₇-C₄₀ saturated alkane mixture, sodium bicarbonate, tetrahydrofuran (THF), triisopropylsilyl chloride (TIPSCl). NMR solvents were acquired either from Sigma Aldrich™ or Merck KGaA™.

All reactions, sensitive to air or moisture, were carried out in oven or flame-dried glassware under positive pressure of argon using standard Schlenk techniques. SiliaFlash™ G60 silica gel was used for column chromatography. Thin-layer chromatography (TLC) was performed using Merck Kieselgel 60 F254 TLC™ plates. Compounds were visualized by UV (λ=254 nm, 366 nm), iodine vapours and/or p-anisaldehyde, phosphomolybdic acid or KMnO₄ stain solutions.

The GC/MS analyses were carried out using an Agilent 5977A™ mass selective detector (MSD) coupled to 7890B™ gas chromatograph (equipped with a HP-5MS fused-silica capillary column: 5% phenylmethylsiloxane; 30 m×0.25 mm, film thickness, 0.25 μm; Agilent Technologies™); the MSD was operated at the ionization energy of 70 eV; retention indices were determined relative to a standard series of n-alkanes (C₇-C₄₀). Low resolution ESIMS was recorded on Bruker HCTultra PTM Discovery System™ and high resolution ESIMS was recorded on a Waters/Micromass LCT™ time-of-flight (TOF) mass spectrometer equipped with an electrospray ion source or Kratos Concept IIHQ™. Pure samples of alotaketal analogues were obtained from reversed-phase HPLC using an InertSustain C18™, 5 μm, 10×250 mm column with MaCN/H₂O (3:1) or MeCN as eluent.

All NMR spectra (¹H, ¹³C, ³¹P, DEPT-135, ¹H-¹H COSY, gHMQC, gHMBC, and NOESY) were recorded at 25° C. in the following solvents: CDCl₃, CD₂Cl₂ or C₆D₆. Chemical shifts are reported in ppm (δ) and referenced to residual non deuterated solvents. Scalar couplings are reported in Hz. ¹H and ¹³C NMR spectra were recorded on a: a) Bruker Avance 300 MHz Spectrometer™ equipped with a QNP 5 mm probe head (auto-switchable quad nucleus probe for observing ¹H, ¹³C, ³¹P, and 19F), b) Bruker Avance 400 MHz Spectrometer™ equipped with a Bruker 5 mm BBI™ (inverse broadband probe with Z-gradient coil tunable from ¹⁰⁹Ag to ³¹P) or c) Bruker Avance 600 MHz Spectrometer™ equipped with a Bruker z-gradient TCI (¹H, ¹³C, ¹⁵N) cryoprobe. The acquired NMR experiments, both 1D and 2D, were recorded using standard Bruker™ built-in pulse sequences. bs—broad signal, d—doublet, dd—doublet of doublets, ddd—doublet of doublets of doublets, dddt—doublet of doublets of doublets of triplets, ddp—doublet of doublets of pentets, dt—doublet of triplets, (o)m—(overlapped) moultiplet(s), s—singlet.

### (R)-5-(3-Chloroprop-1-en-2-yl)-2-methylcyclohex-2-enone (I1)

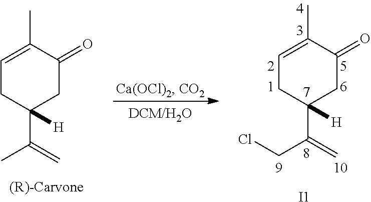

To a vigorously stirred suspension of Ca(OCl)₂ (≈70%, 27.3 g, ≈0.13 mol) in H₂O (130 mL) was added (R)-carvone (30.0 g, 0.2 mol) in DCM (200 mL). Dry ice (ca. 200 g) was added to the mixture portionwise over 7 h before the mixture was filtered. The organic phase was separated, the aqueous phase extracted with DCM (2×150 mL) and the combined organic extracts were dried over anhydrous MgSO₄ and concentrated in vacuo. Purification by column chromatography (SiO₂, isocratic, EtOAc/Hex 1:11) afforded allyl chloride (23.6 g, 64%) as a pale yellow oil and recovered (R)-carvone (4.9 g, 16%).

¹H NMR (600 MHz, Chloroform-d) δ_{H} 6.75 (m, 1H, H-2), 5.25 (s, 1H, H-10a), 5.05 (d, J=1.3 Hz, 1H, H-10b), 4.13-4.03 (m, 2H, H-9), 2.97 (m, 1H, H-7), 2.65 (ddd, J=16.1, 3.8, 1.5 Hz, 1H, H-6β), 2.55 (dddt, J=18.0, 6.0, 3.8, 1.5 Hz, 1H, H-1β), 2.38 (dd, J=16.1, 13.1 Hz, 1H, H-6a), 2.32 (ddp, J=18.0, 10.4, 2.8 Hz, 1H, H-1a), 1.78 (m, 3H, H-4).

¹³C NMR (151 MHz, CDCl₃) δ_{C} 199.0 (C-5), 146.7 (C-8), 144.2 (C-2), 135.8 (C-3), 115.3 (C-10), 47.0 (C-9), 43.2 (C-6), 38.0 (C-7), 31.5 (C-1), 15.8 (C-4).

MS: EIMS (70 eV; relative intensity, %) 184(1, [M·⁺]), 149(23), 142(17), 107(17), 93(32), 82(100), 54(27), 41(8); HRMS (FD+) calcd for C10H13OCl [M+H]⁺ 184.0655, found 184.0655.

Chromatography: Rf 0.49 (EtOAc/Hex, 1:9); RI 1517 (HP-5 MS)

### (R)-5-(3-Hydroxyprop-1-en-2-yl)-2-methylcyclohex-2-enone (I2)

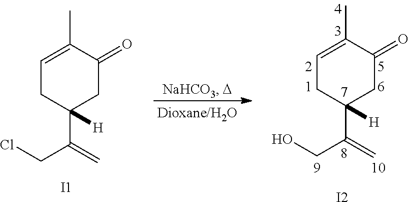

To a stirred solution of allyl chloride I1 (30.0 g, 0.16 mol) in 1,4-dioxane/H₂O (1:3, 600 mL) was added NaHCO₃ (34.2 g, 0.41 mol). The reaction mixture was stirred at reflux for 18 h before being cooled to RT and extracted with DCM (3×500 mL). The combined organic extracts were dried (MgSO₄) and concentrated in vacuo. Purification by column chromatography (SiO₂, isocratic, EtOAc/Hex 1:1 afforded allylic alcohol 12 (20.3 g, 75%) as a pale yellow oil.

¹H NMR (600 MHz, CDCl₃) δ_{H} 6.74 (m, 1H, H-2), 5.14 (d, J=1.6 Hz, 1H, H-10a), 4.94 (d, J=1.1 Hz, 1H, H-10b), 4.14 (bs, 2H, H-9), 2.88-2.76 (m, 1H, H-7), 2.68-2.20 (om, 4H, H-6a, H-6p, H-1a, H-13), 1.80 (bs, 1H, OH), 1.77 (m, 3H, H-4).

¹³C NMR (151 MHz, CDCl₃) δ_{C} 199.6 (C-5), 150.4 (C-8), 144.7 (C-2), 135.5 (C-3), 110.2 (C-10), 64.7 (C-9), 43.3 (C-6), 38.3 (C-7), 31.7 (C-1), 15.7 (C-4).

MS: EIMS (70 eV; relative intensity, %) 166 (6, [M·⁺]), 148(68), 135(19), 106(79), 91(47), 82(100), 54(46), 41(16). HRMS (FD+) calcd for C10H14O₂ [M+H]⁺ 167.1072, found 167.1077.

Chromatography: Rf 0.25 (EtOAc/Hex, 1:1); RI 1557 (HP-5 MS)

### (R)-2-methyl-5-(3-((triisopropylsilyl)oxy)prop-1-en-2-yl)cyclohex-2-en-1-one (I3)

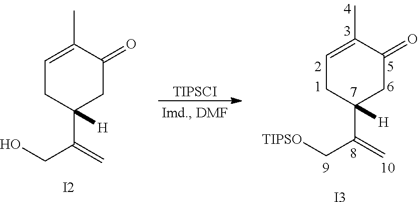

To a solution of 12 (20.0 g, 0.12 mol) in anhydrous DMF (120 mL) was added imidazole (24.6 g, 0.37 mol). The reaction mixture was stirred at 0° C. for 10 min before addition of TIPSCl (34.9 g, 0.18 mol). The reaction was allowed to warm up to RT and stirred for an additional 1.5 h before being quenched and diluted with H₂O (1 L). The mixture was extracted with Et₂O (3×250 mL) and the combined organic extracts were washed with H₂O (250 mL), dried (MgSO₄) and concentrated in vacuo. Purification by flash chromatography (SiO₂, isocratic, EtOAc/Hex 1:10) afforded TIPS ether of 12 (36.7 g, 95%) as a colorless oil.

¹H NMR (600 MHz, CDCl₃) δ_{H} 6.74 (ddd, J=5.8, 2.8, 1.4 Hz, 1H, H-2), 5.19 (q, J=1.6 Hz, 1H, H-10a), 4.90 (p, J=1.3 Hz, 1H, H-10b), 4.21 (bs, 2H, H-9), 2.83-2.70 (m, 1H, H-7), 2.61 (ddd, J=16.0, 3.8, 1.5 Hz, 1H, H-6β), 2.56-2.21 (m, 3H, H-1a, H-13 and H-6a), 1.78 (m, 3H, H-4), 1.21-0.97 (m, 21H, Si(CH(CH₃)₂)₃)).

¹³C NMR (150 MHz, CDCl₃) δ_{C} 199.8 (C5), 150.1 (C8), 144.8 (C2), 135.6 (C3), 109.1 (C10), 65.2 (C9), 43.6 (C6), 38.3 (C7), 31.9 (C1), 18.2 (6C, Si(CH(CH₃)₂)₃), 15.9 (C4), 12.1 (3C, Si(CH(CH₃)₂)₃).

MS: EIMS (70 eV; relative intensity, %) 322 (3, [M·⁺]), 280(99), 279(99), 237(16), 193(12), 165(12), 149(13), 132(12), 131(83), 103(100), 91(27), 75(79), 61(36), 41(11).

HRMS (FD+) calcd for C10H13OCl [M+H]⁺ 323.2406, found 323.2396.

Chromatography: Rf 0.51 (EtOAc/Hex, 1:9); RI 2176 (HP-5 MS).

### (4S,5R)-4-hydroxy-2-methyl-5-(3-((triisopropylsilyl)oxy)prop-1-en-2-yl)cyclohex-2-en-1-one (I4)

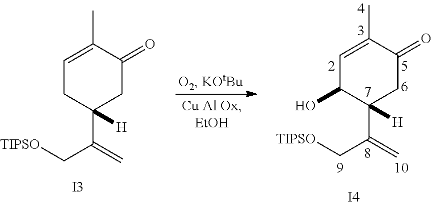

The Cu Al Ox catalyst (2.60 g; prepared as described below) was suspended in absolute ethanol (250 ml) with vigorous stirring for 10 min. The substrate (I3, 10.0 g, 31.0 mmol) dissolved in ethanol (60 ml) and ^{t}BuOK (1.74 g, 15.5 mmol) were added. The mixture was stirred under a stream of oxygen for 20 h. The solvent was removed under vacuum and the resulting oily residue was purified by silica gel column chromatography (mixtures of EtOAc/hexanes, 1:5). The yield of the pure I4 was 30%, while 40% of the starting material was recovered. The yield of I4 was 14% when the reaction was repeated in the absence of a catalyst (all other conditions the same as stated above).

Preparation of the Cu Al Ox catalyst: A solution containing Na₂CO₃ (1.27 g) and NaOH (5.20 g) in water (100 mL) was added dropwise over 1.5 h to a second solution containing CuCl₂ (5.0 g) and AlCl₃·6H₂O (4.0 g) in water (50 mL). The resulting blue suspension was stirred at 70° C. for 22 h. A gradual change in color from blue to black was observed. The black precipitate was filtered, and the residue was washed with warm water. The solid was then dried in the oven at 105° C. for 24 h, after which time it was ground to a uniform consistency. Finally, it was left exposed to air for 3 d before use.

¹H NMR (600 MHz, Chloroform-d) δ_{H} 6.73 (d, J=2.4 Hz, 1H, H-2), 5.29 (s, 1H, H-10a), 5.13 (s, 1H, H-10b), 4.52-4.42 (m, 1H, H-1), 4.27 (d, J=12.0 Hz, 1H, H-9a), 4.19 (d, J=12.0 Hz, 1H, H-9b), 2.73 (ddd, J=12.6, 9.7, 5.6 Hz, 1H, H-7), 2.55-2.44 (m, 2H, H-6), 1.80 (bs, 3H, H-4), 1.26-1.00 (om, 21H, Si(CH(CH₃)₂)₃)).

¹³C NMR (151 MHz, CDCl₃) δ_{C} 198.8 (C-5), 148.3 (C-2), 147.2 (C-8), 134.8 (C-3), 115.7 (C-10), 71.4 (C-1), 66.9 (C-9), 49.1 (C-7), 42.0 (C-6), 18.1 (Si(CH(CH₃)₂)₃), 15.4 (C-4), 12.0 (Si(CH(CH₃)₂)₃).

MS: HRMS (FD+) calcd for C10H13OCl [M+H]⁺ 339.2355, found 339.2366.

Chromatography: Rf 0.54 (EtOAc/Hex, 1:4)

### (1R,6R)-3-methyl-4-oxo-6-(3-((triisopropylsilyl)oxy)prop-1-en-2-yl)cyclohex-2-en-1-yl 2-(diethoxyphosphoryl)acetate (I5)

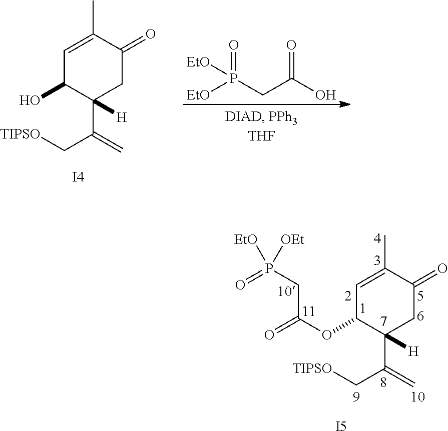

To a solution of 14 (3.38 g, 10.0 mmol), 2-(diethoxyphosphoryl)acetic acid (3.92 g, 20.0 mmol) and triphenylphosphine (5.24 g, 12.7 mmol) in THF (100 mL) was added diisopropyl azodicarboxylate (3-93 mL, 20.0 mmol) dropwise at 0° C. under nitrogen. The resulting mixture was stirred at room temperature for 3 h and then diluted with EtOAc (150 mL), washed with H2O and brine. The organic layer was dried over Na2SO4, concentrated and purified by flash column chromatography (silica gel, eluted with EtOAc/Hex=1:1) to provide product (4.38 g) in 85% yield.

¹H NMR (600 MHz, Chloroform-d) δ_{H} 6.86-6.70 (m, 1H, H-2), 5.54 (dd, J=5.7, 3.2 Hz, 1H, H-1), 5.25 (s, 1H), 4.91 (bs, 1H), 4.22 (m, 2H, H-9), 4.12 (om, 4H, OCH₂CH₃), 3.12 (dt, J=12.9, 3.6 Hz, 1H, H-7), 3.04-2.78 (om, 3H, H-10′ and H-6a), 2.53 (dd, J=16.3, 3.9 Hz, 1H, H-6β), 1.83 (bs, 3H, H-4), 1.31 (om, 6H, OCH₂CH₃), 1.04 (om, 21H, Si(CH(CH₃)₂)₃)).

¹³C NMR (151 MHz, CDCl₃) δ_{C} 199.2 (C-5), 165.4 (C-11), 146.0 (C-8), 139.3 (C-3), 138.2 (C-2), 112.2 (C-10), 67.8 (C-1), 65.7 (C-9), 62.8 (OCH₂CH₃), 62.8 (OCH₂CH₃), 39.8 (C-7), 37.7 (C-6), 35.2 (C-10′, 33.4 (C-10′), 18.1 (Si(CH(CH₃)₂)₃), 16.5 (OCH₂CH₃), 16.4 (OCH₂CH₃), 15.7, 12.1 (Si(CH(CH₃)₂)₃).

MS: HRMS (FD+) calcd for C10H13OCl [M+H]⁺ 517.2750, found 517.2765.

Chromatography: Rf 0.25 (EtOAc/Hex, 1:1)

### (8R,9R)-6-methyl-9-(3-((triisopropylsilyl)oxy)prop-1-en-2-yl)-1,4-dioxaspiro[4.5]dec-6-en-8-yl 2-(diethoxyphosphoryl)acetate (I6)

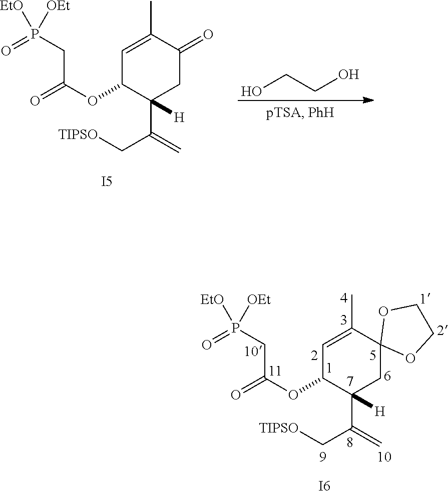

To the benzene (25 ml) solution of 15 (2.0 g, 3.9 mmol) was added ethylene glycol (2.17 mL, 38.7 mmol) and pTSA (50 mg). Reaction mixture was refluxed using Dean-Stark apparatus for 8 h. The reaction mixture was diluted with EtOAc (50 ml) and washed with saturated brine. Water layer was extracted with EtOAc (3×10 ml). Combined organic extracts were dried over MgSO₄ and purified by flash column chromatography (silica gel, eluted with EtOAc/Hex=1:1). As I6 and I5 co-elute under these conditions, if necessary, traces of unreacted 15 can be removed by dissolving (semi)crude product in EtOH and stirring (room temperature, ca. 1 h) with a small amount of NaBH₄. Standard workup and quick column (silica gel, EtOAc/Hex=1:1) afford I6 of sufficient purity for the next step (yield 1.75 g; 80%).

¹H NMR (600 MHz, Chloroform-d) δ_{H} 5.84-5.77 (m, 1H, H-2), 5.35-5.26 (m, 1H, H-1), 5.22 (bs, 1H, H-10a), 4.87 (bs, 1H, H-10b), 4.27-3.84 (om, 10H, H-9, H-1′, H-2′, OCH₂CH₃), 3.02-2.70 (om, 3H, H-10′ and H-7), 2.18-1.99 (m, 1H, H-6a), 1.91-1.78 (m, 1H, H-6β), 1.72 (m, 3H, H-4), 1.32 (om, 6H, OCH₂CH₃), 1.19-0.98 (om, 21H, Si(CH(CH₃)₂)₃)).

¹³C NMR (151 MHz, CDCl₃) δ_{C} 165.4 (C-11), 147.6 (C-8), 141.7 (C-3), 124.8 (C-2), 110.8 (C-10), 107.3 (C-5), 68.0 (C-1), 66.2 (C-9), 65.6, 64.9, 62.8, 62.7 (C-1′, C-2′, OCH₂CH₃), 38.0 (C-7), 35.2 (C-10′), 33.4 (C-10′), 33.2 (C-6), 18.1 (Si(CH(CH₃)₂)₃), 16.5, 16.4, 16.1 (OCH₂CH₃, OCH₂CH₃, C-4), 12.1 (Si(CH(CH₃)₂)₃).

MS: HRMS (FD+) calcd for C10H13OCl [M+H]⁺ 560.2934, found 560.2944.

Chromatography: Rf 0.25 (EtOAc/Hex, 1:1)

### (8R,9S)-6-methyl-9-(2-((triisopropylsilyl)oxy)acetyl)-1,4-dioxaspiro[4.5]dec-6-en-8-yl 2-(diethoxyphosphoryl)acetate (I7)

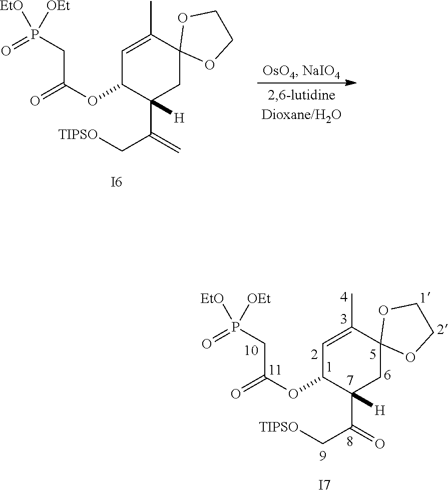

To a stirred solution 16 (1.5 g, 2.68 mmol) in dioxane/H₂O (3:1, 48 mL) was added 2,6-lutidine (0.612 mL, 5.37 mmol), OsO₄ (2.5 wt. % in t-BuOH, 0.701 mL, 53.60 μmol), and NaIO₄ (2.3 g, 10.72 mmol). The reaction mixture was stirred for 3 h before being diluted with H₂O (50 mL) and Na₂S₂O₃ (50 mL). The mixture was stirred for 30 min before being extracted with DCM (3×100 mL) and the combined organic extracts were dried (MgSO₄) and concentrated in vacuo. Purification by flash chromatography (EtOAc/Hex, 1:1) afforded ketone 17 (753.1 mg, 50%).

¹H NMR (300 MHz, Chloroform-d) δ_{H} 5.84 (om, 2H, H-1 and H-2), 4.43-4.17 (m, 2H, H-9), 4.16-3.81 (om, 8H, OCH₂CH₃, H-1′, H-2′), 3.52 (dt, J=12.8, 3.0 Hz, 1H, H-7), 2.86 (dd, J=21.5, 1.4 Hz, 1H), H-1′, 2.26-1.86 (m, 2H, H-6), 1.72 (s, 3H, H-4), 1.32 (om, J, 6H, OCH₂CH₃), 1.22-0.93 (m, 21H, Si(CH(CH₃)₂)₃)).

Chromatography: Rf 0.23 (EtOAc/Hex, 1:1)

### (4aR,8aR)-7-methyl-4-(((triisopropylsilyl)oxy)methyl)-4a,8a-dihydro-2H,5H-spiro[chromene-6,2′-[1,3]dioxolan]-2-one (I8)

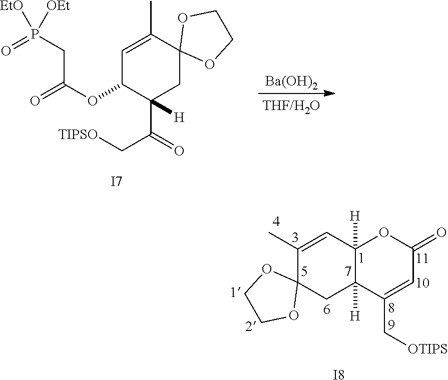

To a stirred solution of I7 (688.0 mg, 1.22 mmol) in THF (50 mL) was added Ba(OH)₂ (420.6 mg, 2.45 mmol) and H₂O (3.1 mL). The reaction was stirred for 2 hours and then quenched with NaHCO₃ (50 mL, saturated solution). The mixture was partitioned between DCM (3×100 mL) and H₂O (80 mL) and the combined organic extracts were dried (MgSO₄) and concentrated in vacuo. After purification by flash chromatography (EtOAc:Hex 1:4) 473.6 mg (95%) of lactone I8 were obtained.

¹H NMR (300 MHz, Chloroform-d) δ_{H} 6.09 (t, J=1.9 Hz, 1H, H-10), 5.81 (m, 1H, H-2), 4.72 (t, J=4.6 Hz, 1H, H-1), 4.41 (d, J=1.9 Hz, 2H, H-9), 4.18-3.86 (om, 4H, H-1′, H-2′), 2.64 (dt, J=12.1, 4.1 Hz, 1H, H-7), 2.00-1.74 (om, 5H, H-6a, 63 and H-4), 1.29-0.95 (om, 21H, Si(CH(CH₃)₂)₃)).

Chromatography: Rf 0.38 (EtOAc/Hex, 1:4)

### General Procedure for Preparation of Bromides, I9a-e

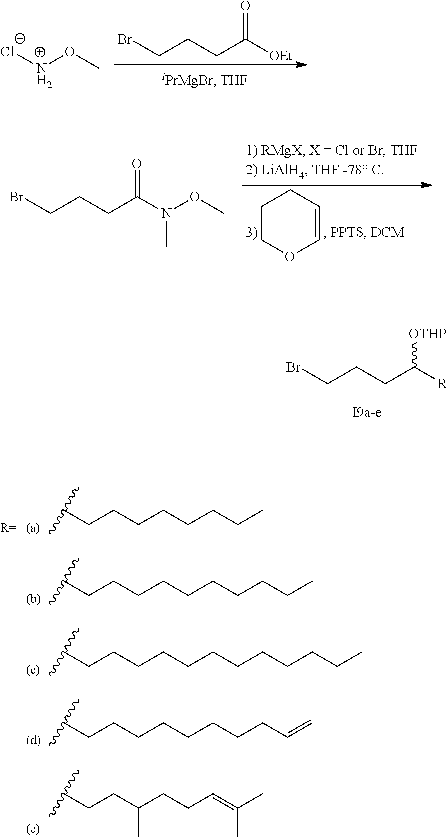

Modified procedure of Prosser and Liotta (Prosser and Liotta 2015) was used for the synthesis of 3-bromopropyl alkyl ketones. Ethyl 4-bromobutanoate (1, 2.726 g, 14.0 mmol, 2 ml) was dissolved in dry THF (40 mL) and added to a flame dried 100 mL round bottom flask containing N,O-dimethylhydroxylamine hydrochloride (1.639 g, 16.8 mmol, 1.2 equiv). After the mixture was being cooled to 0° C., 2M THF solution of isopropylmagnesium chloride (16.8 mL, 33.6 mmol, 2.4 equiv) was added dropwise and then stirred for an additional 30 min. During this time, after initial formation of precipitate, the reaction mixture becomes clear. After in situ generation of intermediate Weinreb amide was completed (this was confirmed by GC-MS analysis) 1 equiv of RMgBr or RMgCl (e.g. allylmagnesium bromide 14 mL, 14 mmol, 1M in THF; prepared from appropriate bromides by standard procedure) was added drop wise and the reaction mixture was allowed to stir for approximately one hour. The progress of the reaction was followed by TLC and GC-MS. Prolonged reaction times lead to decrease of the yield. Overnight stirring of the reaction mixture results in complete loss of the target ketones.

Upon completion, the reaction was quenched with a slow addition of saturated aqueous NH₄Cl (30 mL; this should be added slowly) and allowed to stir for 10 min. The mixture was partitioned with EtOAc and separated. The aqueous layer was extracted with EtOAc three more times. The combined organic layers were dried over anhydrous magnesium sulfate. Yield of crude ketones ranged from 40-60%. They were immediately subjected to reduction with LiAlH₄, without any purification (ketones were unstable upon storage at −20° C., and, to some extent, in contact with SiO₂).

Ketone was dissolved in dry THF and cooled to −78° C. LiAlH₄ (1 mole equivalent) was added in portions, slowly. Progress of reaction was followed by TLC. Reaction was completed after 10-20 min (prolonged reaction times decreased yield significantly) and then quenched with saturated NH₄Cl solution and extracted with diethyl ether. Combined organic fractions were dried over anhydrous magnesium sulfate and solvent is removed under vacuum. Crude products are quickly passed through a small pad of SiO₂ column (EtOAc:Hex=1:4). After removal of solvent, bromo alcohols (1 mol equivalent) were dissolved in dry DCM (8 ml per 1 mmol of alcohol); dihydropyran (2 mol equivalents), and PPTS (0.1 mol equivalent) were added and the reaction mixture was stirred at room temperature for 16 h under N₂. The mixture was diluted with ether (10 mL per 1 mmol of alcohol) and washed with water and saturated brine. Crude products were purified by flash chromatography (SiO₂ column, from Hex to EtOAc:Hex=1:15). Combined yields over 4 steps were in 20-30% range.

### 2-((1-Bromododecan-4-yl)oxy)tetrahydro-2H-pyran, mixture of diastereomers (I9a)

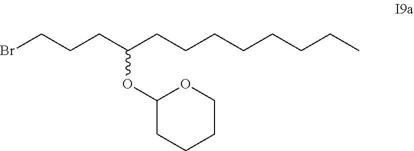 [SMILES: CCCCCCCCC(CCCBr)OC1CCCCO1]

¹H NMR (600 MHz, Chloroform-d) δ 4.71-4.54 (m, 1H), 3.98-3.79 (m, 1H), 3.73-3.55 (m, 1H), 3.54-3.35 (m, 3H), 2.12-1.00 (m, 24H), 0.87 (t, J=6.5 Hz, 3H).

¹³C NMR (151 MHz, Benzene-d₆) δ_{C} 97.9, 97.7, 94.8, 76.1, 75.8, 71.3, 63.2, 62.8, 37.8, 35.9, 35.0, 34.5, 34.3, 34.2, 33.9, 33.5, 32.0, 31.4, 30.8, 30.0, 29.9, 29.7, 29.4, 29.4, 29.2, 29.1, 28.4, 25.7, 25.6, 25.2, 22.8, 20.3, 20.0, 14.2.

MS: HRMS (FD+) calculated for C17H34BrO2 [M+H]⁺ 349.1737, found 349.1749.

### 2-((1-Bromotetradecan-4-yl)oxy)tetrahydro-2H-pyran, mixture of diastereomers (I9b)

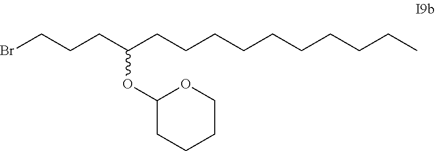 [SMILES: CCCCCCCCCCC(CCCBr)OC1CCCCO1]

¹H NMR (300 MHz, Chloroform-d) δ 4.71-4.54 (m, 1H), 3.98-3.79 (m, 1H), 3.73-3.55 (m, 1H), 3.54-3.35 (m, 3H), 2.12-1.00 (m, 28H), 0.87 (t, J=6.5 Hz, 3H).

MS: HRMS (FD+) calculated for C19H38BrO2 [M+H]⁺ 377.2050, found 377.2055.

### 2-((1-Bromohexadecan-4-yl)oxy)tetrahydro-2H-pyran, mixture of diastereomers (I9c)

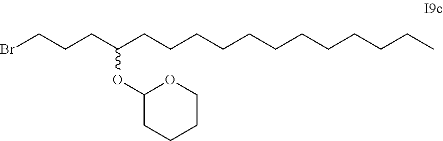 [SMILES: CCCCCCCCCCCCC(CCCBr)OC1CCCCO1]

¹H NMR (300 MHz, Chloroform-d) δ 4.71-4.54 (m, 1H), 3.98-3.79 (m, 1H), 3.73-3.55 (m, 1H), 3.54-3.35 (m, 3H), 2.12-1.00 (m, 32H), 0.87 (t, J=6.5 Hz, 3H).

MS: HRMS (FD+) calculated for C21H42BrO2 [M+H]⁺ 405.2363, found 405.2371.

### 2-((1-Bromotetradec-13-en-4-yl)oxy)tetrahydro-2H-pyran, mixture of diastereomers (I9d)

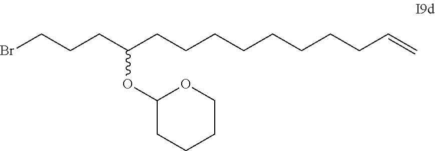 [SMILES: C=CCCCCCCCCC(CCCBr)OC1CCCCO1]

¹H NMR (300 MHz, Chloroform-d) δ 5.81-5.77 (m, 1H), 5.09-4.89 (m, 2H), 4.70-4.52 (m, 1H), 4.00-3.78 (m, 1H), 3.72-3.54 (m, 1H), 3.54-3.35 (m, 3H), 2.30-1.00 (m, 26H).

MS: HRMS (FD+) calculated for C19H36BrO2 [M+H]⁺ 375.1893, found 377.1881.

### 2-((1-Bromo-7,11-dimethyldodec-10-en-4-yl)oxy)tetrahydro-2H-pyran, mixture of diastereomers (I9e)

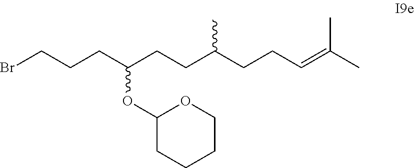 [SMILES: CC(C)=CCCC(C)CCC(CCCBr)OC1CCCCO1]

¹H NMR (300 MHz, Chloroform-d) δ 5.26-5.12 (m, 1H), 4.70-4.52 (m, 1H), 4.00-3.78 (m, 1H), 3.72-3.54 (m, 1H), 3.54-3.35 (m, 3H), 1.97-1.38 (m, 25H), 0.96 (d, J=6.5 Hz, 3H)

MS: HRMS (FD+) calculated for C19H36BrO2 [M+H]⁺ 375.1893, found 377.1986.

### 2-(4-Bromobutoxy)tetrahydro-2H-pyran, I9f

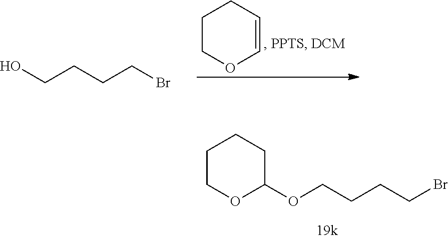 [SMILES: OCCCCBr ; C1=COCCC1 ; BrCCCCOC1CCCCO1]

Bromo alcohol (1.0 g, 6.5 mmol) was dissolved in 30 ml of dry DCM; dihydropyran (1.1 g, 13.0 mmol), and PPTS (81 mg, 0.3 mmol) were added and the reaction mixture was stirred at room temperature for 16 h under N₂. The mixture was diluted with ether (50 ml) and washed with water and saturated brine. Crude product was purified by flash chromatography (SiO₂ column, from Hex to EtOAc:Hex=1:15). Yield of the purified product was 70%.

¹H NMR (300 MHz, Chloroform-d) δ 5.47-4.49 (m, 1H), 3.86-3.66 (m, 2H), 3.51-3.33 (m, 4H), 2.02-1.86 (m, 2H), 1.85-1.59 (m, 4H), 1.58-1.40 (m, 4H).

¹³C NMR (151 MHz, Benzene-d₆) δ_{C} 99.30, 66.87, 62.64, 34.54, 31.31, 30.52, 28.98, 26.14, 20.18.

MS: HRMS (FD+) calculated for C9H18BrO2 [M+H]⁺ 237.0485, found 237.0482.

General Procedure for the Preparation of Alotaketale Analogues from Lactone I8 and Bromides I9a-k.

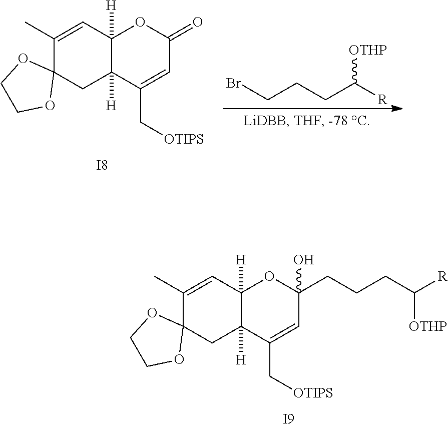

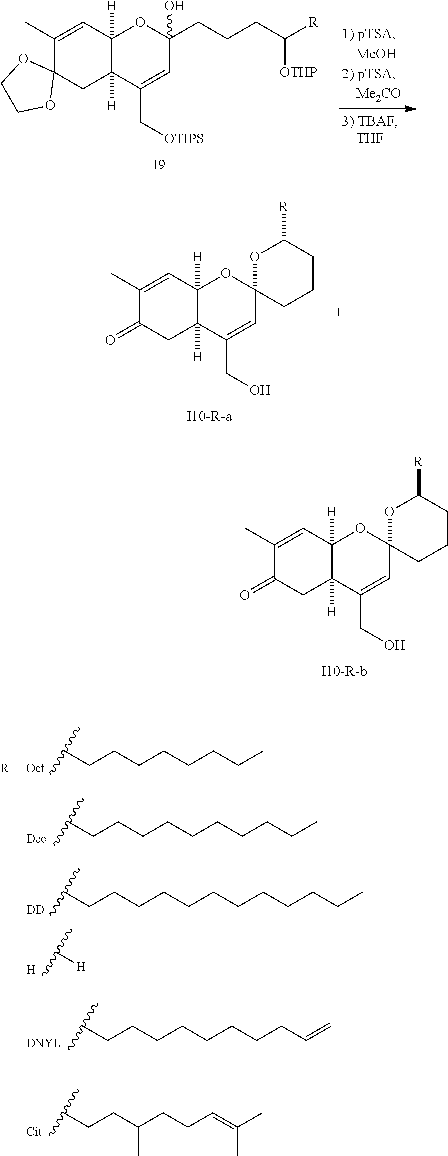

Preparation of LiDBB: 724 mg of DBB was placed in a flame dried round bottom ask. Pre-cut lithium metal was added under argonne atmosphere (glove bag was used). After the addition of 5.42 mL of dry THF, reaction mixture was stirred (glass coated magnetic stir bar) under argonne, at 0° C., for 2 h before use.

To a stirred solution of lactone 18 (1 eq) and appropriate bromide (I9a-f, 1.2 eq) in THF (1 mL per 25 mol of 18) at −78° C. was added sufficient LiDBB (≈6 eq; ≈12 μL per mol of 18) dropwise over 5 min, until a deep green color persisted. The mixture was then stirred at −78° C. for an additional 5 min before being quenched with saturated NH4Cl (≈60 μL per μmol of I8). The mixture was partitioned between DCM (3 times, ≈300 μL per μmol of I8) and H₂O (≈100 μL per μmol of I8) and the combined organic extracts were dried (MgSO₄) and concentrated in vacuo. Crude product was dissolved in a small amount of hexanes and applied on the top of a short silica column. Column was first washed with hexanes (≈200 μL per μmol of I8). Target hemiacetales (I9; mixture of diastereomers) were eluted with (SiO₂,EtOAc/Hex, 1:4). The solvent was removed and the product immediately subjected to the next step, without any additional purification. The yields of semi-pure products were typically 50%.

Semi-pure I9 was dissolved in MeOH, cooled down to −78° C. and then, a catalytic amount of pTSA was added. The reaction mixture was stirred for approximately 3 h, during which time it was allowed to slowly warm up to rt. The progress of the reaction was followed by TLC. When the TLC spot that corresponds to a starting I9 was no longer observable, almost all MeOH was removed under a gentle stream of nitrogen and the residue was re-dissolved in acetone. The stirring continued for an additional 3 h and then concentrated under vacuum. The residue was diluted with EtOAc and passed through a short pad of Na₂CO₃. Solvent was removed under vacuum, residue was dissolved in dry THF (1 mL per 10 μmol of crude 19) and TBAF (4 eq; 1M in THF) was added. Reaction mixture was stirred for 3 h at room temperature after which time it was diluted with EtOAc, washed with saturated NH₄Cl and dried over MgSO₄. Crude product was first purified by flash chromatography (SiO₂,EtOAc/Hex, 1:2). Final purification was carried out by reverse phase HPLC (MeCN/H₂O (3:1) or MeCN, 2 mL·min, λ=248 nm). Typical yields for I10 (over 3 steps from I9) were 70% (for mixture of epimers at C-15, where applicable). Ratio of a:b isomer (isolated yield) were from 100% a to 60% a:40% b.

### (2S,4aR,6′R/S,8aR)-6′-alkyl-4-(hydroxymethyl)-7-methyl-3′,4a,4′,5′,6′,8a-hexahydrospiro[chromene-2,2′-pyran]-6(5H)-one (I10-H, I10-oct, I10-Dec01, I10-Dec02, I10-Cit3, I1-Cit4, I10-dd)

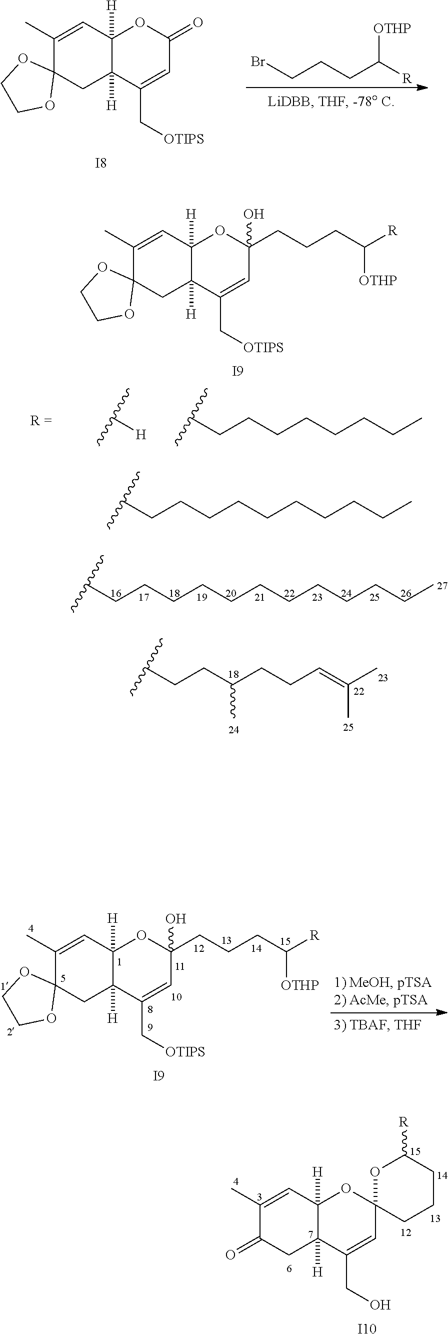

Preparation of LiDBB: 724 mg of DBB was placed in a flame dried round bottom flask. Pre-cut lithium metal was added under argonne atmosphere (glove bag was used). After the addition of 5.42 mL of dry THF, reaction mixture was stirred (glass coated magnetic stir bar) under argonne, at 0° C., for 2 h before use.

To a stirred solution of lactone 18 (1 eq) and appropriate bromide (1.2 eq) in THF (1 mL per 25 μmol of 18) at −78° C. was added sufficient LiDBB (≈6 eq; ≈12 μL per mol of 18) dropwise over 5 min, until a deep green color persisted. The mixture was then stirred at −78° C. for an additional 5 min before being quenched with saturated NH4Cl (≈60 μL per mol of I8). The mixture was partitioned between DCM (3 times, ≈300 μL per μmol of 18) and H₂O (≈100 μL per μmol of 18) and the combined organic extracts were dried (MgSO₄) and concentrated in vacuo. Crude product was dissolved in a small amount of hexanes and applied on the top of a short silica column. Column was first washed with hexanes (≈200 μL per μmol of I8). Target hemiacetales (I9; mixture of diastereomers) were eluted with (SiO₂,EtOAc/Hex, 1:4). The solvent was removed and the product immediately subjected to the next step, without any additional purification. The yields of semi-pure products (I9-H, I9-Oct, I9-Dec, I9-DD or I9-Cit) were typically 50%.

Semi-pure I9 was dissolved in MeOH, cooled down to −78° C. and then, a catalytic amount of pTSA was added. The reaction mixture was stirred for approximately 3 h, during which time it was allowed to slowly warm up to rt. The progress of the reaction was followed by TLC. When the TLC spot that corresponds to a starting I9 was no longer observable, almost all MeOH was removed under a gentle stream of nitrogen and the residue was re-dissolved in acetone. The stirring continued for an additional 3 h and then concentrated under vacuum. The residue was diluted with EtOAc and passed through a short pad of Na₂CO₃. Solvent was removed under vacuum, residue was dissolved in dry THF (1 mL per 10 μmol of crude 19) and TBAF (4 eq; 1M in THF) was added. Reaction mixture was stirred for 3 h at room temperature after which time it was diluted with EtOAc, washed with saturated NH4Cl and dried over MgSO4. Crude product was first purified by flash chromatography (SiO₂, EtOAc/Hex, 1:2). Final purification was carried out by reverse phase HPLC (MeCN/H2O (3:1) or MeCN, 2 mL·min, λ=248 nm). Typical yields for I10 (over 3 steps from 19) were 70% (for mixture of epimers at C-15, where applicable).

### I10-H: (2S,4aR,8aR)-4-(hydroxymethyl)-7-methyl-3′,4a,4′,5′,6′,8a-hexahydrospiro[chromene-2,2′-pyran]-6(5H)-one

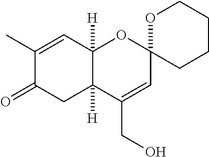

¹H NMR (300 MHz, Methylene Chloride-d₂) δ_{H} 6.77-6.66 (m, 1H, H-2), 5.64 (s, 1H, H-11), 4.41 (dd, J=5.8, 3.2 Hz, 1H, H-1), 4.10 (d, J=1.6 Hz, 2H, H-9), 3.88 (m, 1H, H-15a), 3.61 (m, 1H, H-15b), 2.62-2.30 (m, 3H, H-6a, 6P, H-7), 1.83 (m, 3H, H-4), 1.74-1.45 (m, 6H).

### I10-Oct: (2S,4aR,6′R,8aR)-4-(hydroxymethyl)-7-methyl-6′-octyl-3′,4a,4′,5′,6′,8a-hexahydrospiro[chromene-2,2′-pyran]-6(5H)-one

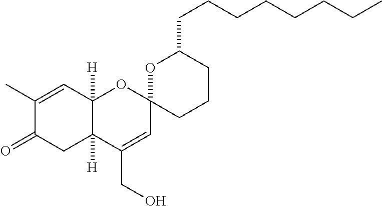

¹H NMR (600 MHz, Benzene-d₆) δ_{H} 6.46-6.35 (m, 1H, H-2), 5.55 (d, J=1.7 Hz, 1H, H-11), 4.37 (dd, J=5.7, 3.4 Hz, 1H, H-1), 3.91 (dddt, J=10.4, 8.2, 4.3, 2.1 Hz, 1H, H-15), 3.59-3.43 (m, 2H, H-9), 2.58-2.34 (m, 2H), 2.17-1.96 (m, 2H), 1.81 (d, J=1.2 Hz, 3H, H-4), 1.73-1.57 (m, 3H), 1.57-1.17 (m, 16H), 0.90 (t, J=6.9 Hz, 3H), 0.46 (t, J=6.0 Hz, 1H, OH).

### I10-Dec01: (2S,4aR,6'S,8aR)-4-(hydroxymethyl)-7-methyl-6′-decyl-3′,4a,4′,5′,6′,8a-hexahydrospiro[chromene-2,2′-pyran]-6(5H)-one

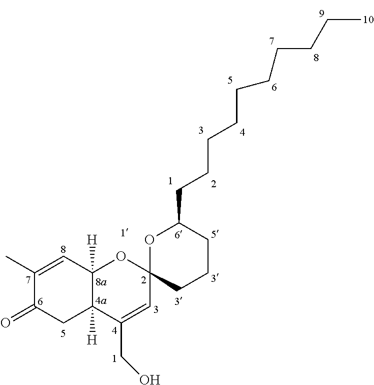 [SMILES: CCCCCCCCCC[C@H]1CCC[C@]2(C=C(CO)[C@H]3CC(=O)C(C)=C[C@H]3O2)O1]

¹H NMR (600 MHz, Benzene-d₆) δ_{H} 6.50 (dt, J=3.1, 1.4 Hz, 1H, H-2), 5.60 (q, J=1.7 Hz, 1H, H-11), 4.27 (ddd, J=5.1, 3.5, 1.9 Hz, 1H, H-1), 4.02 (dddd, J=11.9, 7.4, 4.7, 2.3 Hz, 1H, H-15), 3.73-3.52 (m, 2H, H-9), 2.74 (dd, J=15.9, 6.6 Hz, 1H), 2.55-2.43 (m, 1H), 2.19 (dd, J=15.9, 5.0 Hz, 1H), 2.00 (qt, J=14.1, 4.0 Hz, 1H), 1.84 (m, 3H, H-4), 1.79-1.65 (m, 1H), 1.65-1.54 (m, 2H), 1.54-1.14 (m, 20H), 0.91 (t, J=7.0 Hz, 3H, H-25), 0.41 (dd, J=6.7, 4.6 Hz, 1H, OH).

¹³C NMR (151 MHz, Benzene-d₆) δ_{C} 196.6, 144.6, 138.0, 135.8, 127.0, 119.8, 95.7, 70.5, 69.2, 63.5, 39.1, 37.4, 35.5, 34.6, 32.3, 31.3, 30.5, 30.1, 30.1, 29.8, 26.3, 23.1, 19.3, 15.9, 14.4.

### I10-Dec02: (2S,4aR,6′R,8aR)-4-(hydroxymethyl)-7-methyl-6′-decyl-3′,4a,4′,5′,6′,8a-hexahydrospiro [chromene-2, 2′-pyran]-6 (5H)-one

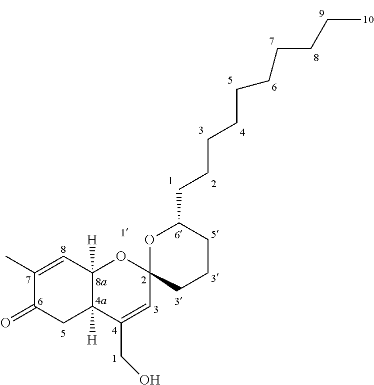 [SMILES: CCCCCCCCCC[C@@H]1CCC[C@]2(C=C(CO)[C@H]3CC(=O)C(C)=C[C@H]3O2)O1]

¹H NMR (600 MHz, Benzene-d₆) δ_{H} 6.46-6.35 (m, 1H, H-2), 5-55 (d, J=1-7 Hz, 1H, H-11), 4-37 (dd, J=5-7, 3-4 Hz, 1H, H-1), 3-91 (dddt, J=10.4, 8.2, 4-3, 2.1 Hz, 1H, H-15), 3.59-3.43 (m, 2H, H-9), 2.58-2.34 (m, 2H), 2.17-1.96 (m, 2H), 1.81 (d, J=1.2 Hz, 3H, H-4), 1.73-1.57 (m, 3H), 1.57-1.17 (m, 20H), 0.90 (t, J=6.9 Hz, 3H), 0.46 (t, J=6.0 Hz, 1H, OH).

¹³C NMR (151 MHz, Benzene-d₆) δ_{C} 197.5, 141.5, 139.7, 138.6, 125.7, 95.7, 70.1, 63.5, 62.8, 38.4, 36.9, 34.9, 34.0, 32.3, 31.3, 30.3, 30.2 (2C), 30.1, 29.8, 26.2, 23.1, 19.4, 16.2, 14.4.

### I10-DD2: (2S,4aR,6′R,8aR)-4-(hydroxymethyl)-7-methyl-6′-dodecyl-3′,4a,4′,5′,6′,8a-hexahydrospiro[chromene-2,2′-pyran]-6(5H)-one

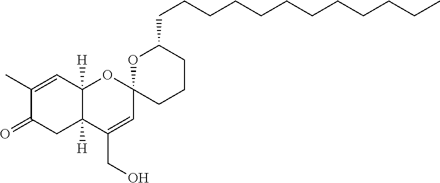 [SMILES: CCCCCCCCCCCC[C@@H]1CCC[C@]2(C=C(CO)[C@H]3CC(=O)C(C)=C[C@H]3O2)O1]

¹H NMR (300 MHz, Benzene-d₆) δ_{H} 6.39 (dd, J=5.7, 1.6 Hz, 1H, H-2), 5.56 (d, J=1.7 Hz, 1H, H-11), 4.37 (dd, J=5.7, 3.4 Hz, 1H, H-1), 4.04-3.80 (m, 1H, H-15), 3.52 (m, 2H, H-9), 2.58-2.29 (m, 2H), 2.19-1.74 (m, 2H), 1.71-1.56 (m, 3H, H-4), 1.56-1.17 (m, 27H), 0.91 (td, J=6.4, 4.1 Hz, 3H, H-27), 0.43 (s, 1H, OH).

### I10-DNYL-a: (2S,4aR,6′R,8aR)-6′-(dec-9-en-1-yl)-4-(hydroxymethyl)-7-methyl-3′,4a,4′,5′,6′,8a-hexahydrospiro[chromene-2,2′-pyran]-6(5H)-one

### I10-DNYL-a OH

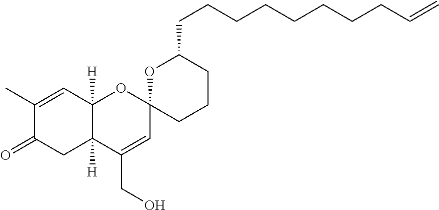 [SMILES: C=CCCCCCCCC[C@@H]1CCC[C@]2(C=C(CO)[C@H]3CC(=O)C(C)=C[C@H]3O2)O1]

¹H NMR (600 MHz, Benzene-d₆) δ_{H} 6.39 (dq, J=5.8, 1.5 Hz, 1H), 5.79 (ddt, J=17.0, 10.2, 6.7 Hz, 1H), 5.55 (q, J=1.3 Hz, 1H), 5.09-4.89 (m, 2H), 4.41-4.34 (m, 1H), 3.90 (dddd, J=11.7, 8.1, 4.1, 2.2 Hz, 1H), 3.60-3.46 (m, 2H), 2.49 (dd, J=15.8, 4.4 Hz, 1H), 2.40 (dd, J=15.8, 13.1 Hz, 1H), 2.13 (dt, J=13.1, 4.0 Hz, 1H), 2.08-1.94 (m, 3H), 1.81 (t, J=1.2 Hz, 3H), 1.72-1.56 (m, 3H), 1.55-1.42 (m, 5H), 1.42-1.15 (m, 11H), 0.49 (t, J=5.9 Hz, 1H). ¹³C NMR (151 MHz, Benzene-d₆) δ_{C} 197.5, 141.5, 139.6, 139.2, 138.6, 125.7, 114.6, 95.7, 70.2, 63.5, 62.8, 38.4, 36.9, 34.9, 34.2, 34.0, 31.2, 30.3, 30.1, 30.0, 29.5, 29.3, 26.2, 19.4, 16.2.

MS: HRMS (FD+) calculated for C25H39O4 [M+H]⁺ 403.5825, found 403.5834.

### I10-DNYL-b: (2S,4aR,6'S,8aR)-6′-(dec-9-en-1-yl)-4-(hydroxymethyl)-7-methyl-3′,4a,4′,5′,6′,8a-hexahydrospiro[chromene-2,2′-pyran]-6(5H)-one

### I10-DNYL-b

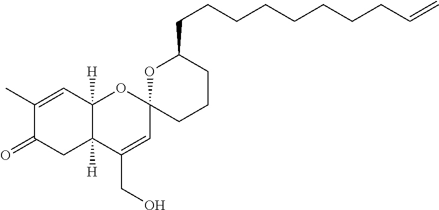 [SMILES: C=CCCCCCCCC[C@H]1CCC[C@]2(C=C(CO)[C@H]3CC(=O)C(C)=C[C@H]3O2)O1]

¹H NMR (600 MHz, Benzene-d₆) δ_{H} 6.49 (dt, J=3.2, 1.4 Hz, 1H), 5.79 (ddt, J=16.9, 10.2, 6.7 Hz, 1H), 5.60 (q, J=1.7 Hz, 1H), 5.10-4.96 (m, 2H), 4.27 (ddq, J=5.3, 3.7, 1.8 Hz, 1H), 4.02 (dddd, J=11.7, 7.4, 4.7, 2.3 Hz, 1H), 3.70 (dd, J=13.8, 6.1 Hz, 1H), 3.59 (dd, J=13.9, 3.8 Hz, 1H), 2.74 (dd, J=15.9, 6.7 Hz, 1H), 2.52 (q, J=5.6 Hz, 1H), 2.19 (dd, J=15.9, 5.0 Hz, 1H), 2.08-1.92 (m, 3H), 1.83 (t, J=1.6 Hz, 3H), 1.75-1.65 (m, 1H), 1.57 (dtd, J=14.4, 7.6, 5.1 Hz, 1H), 1.53-1.12 (m, 17H), 0.47-0.41 (m, 1H).

¹³C NMR (151 MHz, Benzene-d₆) δ_{C} 196.6, 144.6, 139.3, 138.0, 135.8, 129.5, 128.2, 128.1, 127.9, 126.9, 114.5, 95.7, 70.5, 69.1, 63.5, 39.1, 37.3, 35.5, 34.6, 34.2, 31.3, 30.4, 30.0, 29.9, 29.5, 29.3, 26.2, 19.3, 15.9.

MS: HRMS (FD+) calculated for C25H39O4 [M+H]⁺ 403.5825, found 403.5836.

### I10-Cit-a: (2S,4aR,6'S,8aR)-6′-(3,7-Dimethyloct-6-en-1-yl)-4-(hydroxymethyl)-7-methyl-3′,4a,4′,6′,8a-hexahydrospiro[chromene-2,2′-pyran]-6(5H)-one

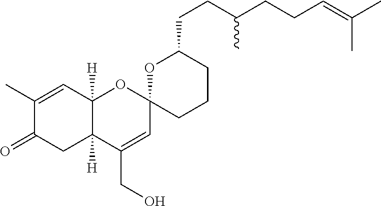 [SMILES: CC(C)=CCCC(C)CC[C@@H]1CCC[C@]2(C=C(CO)[C@H]3CC(=O)C(C)=C[C@H]3O2)O1]

I10-Cit-a; mixture of diastereomers

MS: HRMS (FD+) calculated for C25H39O4 [M+H]⁺ 403.5825, found 403.5836.

### I10-Cit-b: (2S,4aR,6′R,8aR)-6′-(3,7-dimethyloct-6-en-1-yl)-4-(hydroxymethyl)-7-methyl-3′,4a,4′,5′,6′,8a-hexahydrospiro[chromene-2,2′-pyran]-6(5H)-one

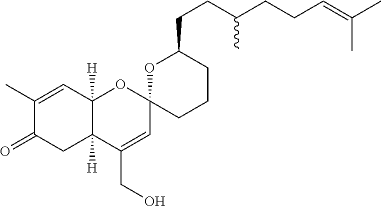 [SMILES: CC(C)=CCCC(C)CC[C@H]1CCC[C@]2(C=C(CO)[C@H]3CC(=O)C(C)=C[C@H]3O2)O1]

I10-Cit-b; mixture of diastereomers

MS: HRMS (FD+) calculated for C25H39O4 [M+H]⁺ 403.5825, found 403.5830.

### I10-Cit3: (2S,4aR,6′R,8aR)-6′-(4,7-dimethyloct-6-en-1-yl)-4-(hydroxymethyl)-7-methyl-3′,4a,4′,5′,6′,8a-hexahydrospiro[chromene-2,2′-pyran]-6(5H)-one

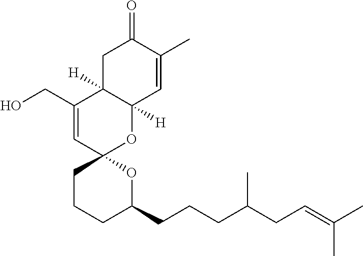 [SMILES: CC(C)=CCC(C)CCC[C@H]1CCC[C@]2(C=C(CO)[C@H]3CC(=O)C(C)=C[C@H]3O2)O1]

¹H NMR (600 MHz, Benzene-d₆) δ_{H} 6.49 (n, 1H, H-2), 5.59 (m, 1H, H-11), 5.19 (dtt, J=17.1, 7.0, 1.5 Hz, 1H, H-21), 4.28 (ddt, J=5.3, 3.7, 2.1 Hz, 1H, H-1), 3.98 (dtd, J=9.6, 4.9, 2.4 Hz, 1H, H-15), 3.64 (m, 2H, H-9), 2.73 (dd, J=15.9, 6.5 Hz, 1H), 2.53 (d, J=5.7 Hz, 1H), 2.18 (dd, J=15.9, 5.0 Hz, 1H), 2.01 (dtt, J=27.6, 13.9, 4.8 Hz, 2H), 1.84 (q, J=1.9 Hz, 3H, H-4), 1.75-1.09 (m, 20H), 0.93-0.80 (m, 3H).

### I10-Cit4: (2S,4aR,6'S,8aR)-6′-(4,7-dimethyloct-6-en-1-yl)-4-(hydroxymethyl)-7-methyl-3′,4a,4′,5′,6′,8a-hexahydrospiro[chromene-2,2′-pyran]-6(5H)-one

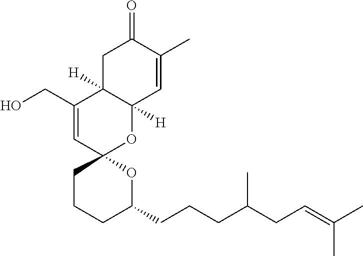 [SMILES: CC(C)=CCC(C)CCC[C@@H]1CCC[C@]2(C=C(CO)[C@H]3CC(=O)C(C)=C[C@H]3O2)O1]

¹H NMR (600 MHz, Benzene-d₆) δ_{H} 6.42-6.32 (m, 1H, H-2), 5.55 (d, J=1.9 Hz, 1H, H-11), 5.26-5.12 (m, 1H, H-21), 4.36 (td, J=6.2, 3.4 Hz, 1H, H-1), 3.87 (ddq, J=11.9, 7.9, 2.4 Hz, 1H, H-15), 3.60-3.36 (m, 2H, H-9), 2.54-2.28 (m, 2H), 2.19-1.95 (m, 4H), 1.81 (dd, J=3.2, 1.4 Hz, 3H, H-4), 1.78-1.54 (m, 8H), 1.54-1.41 (m, 8H), 1.30-1.12 (m, 2H), 0.95 (dd, J=14.2, 6.0 Hz, 3H), 0.45 (td, J=6.0, 2.6 Hz, 1H, OH).

Design and synthesis of Alotaketal C analogs were generated for structure activity evaluations for inhibition of SARS-CoV-2.

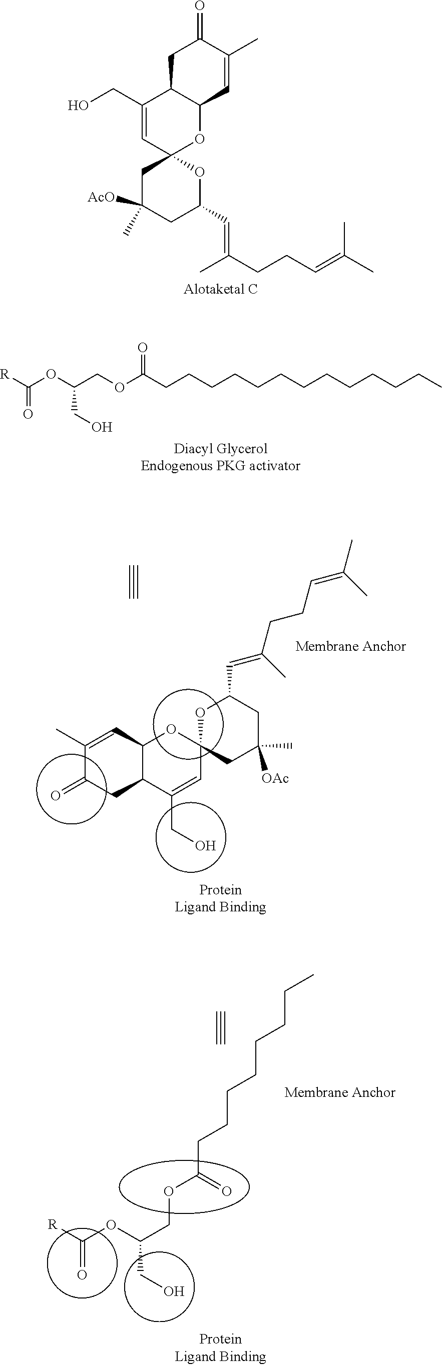

### Example 6: Alotaketal C Derivates Effect on SARS-CoV-2 Omicron Infection

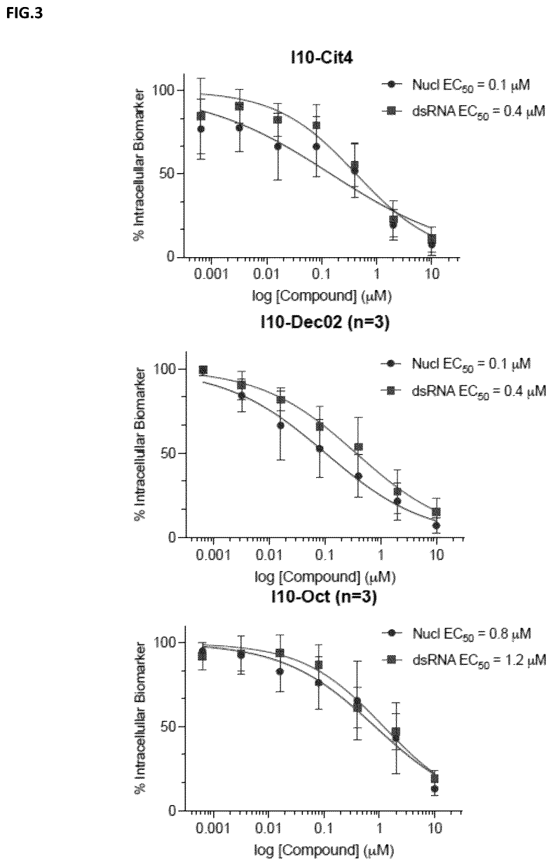

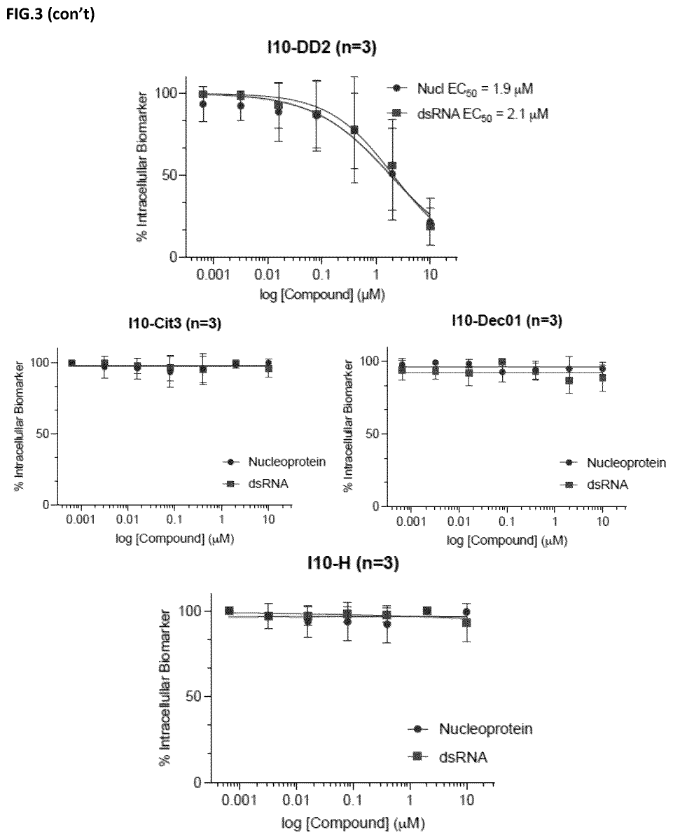

Dose-response curves Alotaketal C derivatives (I10-Cit3, I10-C4, I-Dec01, I10-Dec02, I10-H, I10-Oct, and I10-DD2) generated for Alotaketal C derivatives (I10-Cit3, I10-Cit4, I10-H, and I10-Oct) in Calu-3 cells infected with SARS-CoV-2 Omicron BA.1 testing for viral dsRNA and nucleocapsids using specific antibodies. I10-Dec01, I10-Cit3, and I10-H showed no inhibition activity on SARS-CoV-2 Omicron, at the concentrations tested, but I10-Cit4 I10-Dec02, I10-DD2, and I10-Oct all showed good inhibition based on both dsRNA and nucleocapsids as indicators of viral load (see FIG. 3 and TABLE 2), similar to Alotaketal C. Furthermore, the potency of I10-Cit4 I10-Dec02, I10-DD2, and I10-Oct was comparable to, and in some cases better than, that of Alotaketal C in Calu-3 cells infected with SARS-CoV-2 Omicron BA.1.

TABLE 2
Summary of Alotaketal C Synthetic Analogs

| Compounds | EC₅₀ BA.1 (μM) | CC₅₀ (μM) | SI |
|---|---|---|---|
| Alotaketal C | 0.55 | 100 | 181.818182 |
| I10-Oct | 1 | 100.6 | 100.6 |
| I10-H | 0 | 100 | NA |
| I10-Cit3 | 0 | 100 | NA |
| I10-Cit4 | 0.25 | 12718 | 50872 |
| I10-DD2 | 2 | 113.4 | 56.7 |
| I10-Dec01 | 0 | 339.4 | NA |
| I10-Dec02 | 0.25 | 74.3 | 297.2 |

EC₅₀ = drug concentration required to reduce infection by 50%
CC₅₀ = concentration of test compounds required to reduce cell viability by 50%.
SI = selectivity index

Furthermore, the Alotaketal C analogs having X¹ as ═O; R¹X² as OH; R² and R³ both as H, showed variability with changes of chirality at the R⁴ position as shown in TABLE 3. In particular, when the compound shares R⁴ chirality with Alotaketal C

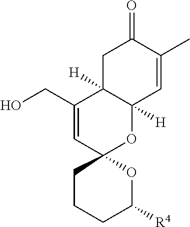

the potency is similar or better than that of Alotaketal C in Calu-3 cells infected with SARS-CoV-2 Omicron BA.1 (i.e. I10-Cit4, I10-Dec02, I10-Oct, and I10-DD2). However, when the opposite chirality was adopted

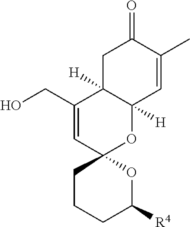

or where R⁴ lacks an alkyl chain potency was decreased in Calu-3 cells infected with SARS-COV-2 Omicron BA.1 (i.e. I10-Cit3, I10-Dec01, and I10-H).

TABLE 3
Structure Activity Comparisons

| | | SARS-COV-₂ |
|---|---|---|
| Compound | Structure | Activity |
| Alotaketal C | 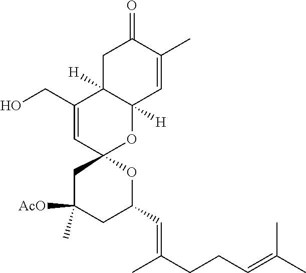 [SMILES: CC(=O)O[C@]1(C)C[C@@H](/C=C(\C)CCC=C(C)C)O[C@@]2(C=C(CO)[C@H]3CC(=O)C(C)=C[C@H]3O2)C1] | yes |
| I₁₀-Oct | 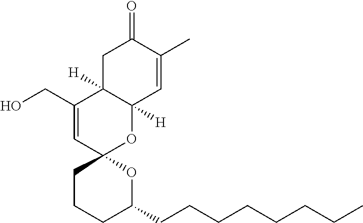 [SMILES: CCCCCCCC[C@@H]1CCC[C@]2(C=C(CO)[C@H]3CC(=O)C(C)=C[C@H]3O2)O1] | yes |
| I₁₀-Cit₄ | 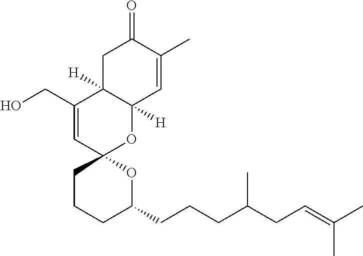 [SMILES: CC(C)=CCC(C)CCC[C@@H]1CCC[C@]2(C=C(CO)[C@H]3CC(=O)C(C)=C[C@H]3O2)O1] | yes |
| I₁₀-DD₂ | 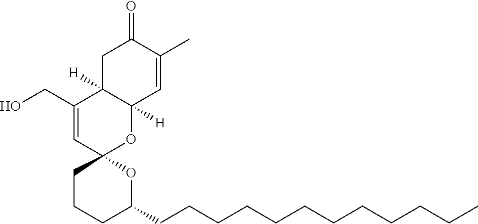 [SMILES: CCCCCCCCCCCC[C@@H]1CCC[C@]2(C=C(CO)[C@H]3CC(=O)C(C)=C[C@H]3O2)O1] | yes |
| I₁₀-Deco₂ | 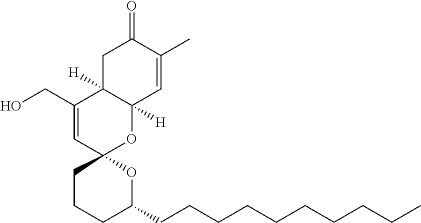 [SMILES: CCCCCCCCCC[C@@H]1CCC[C@]2(C=C(CO)[C@H]3CC(=O)C(C)=C[C@H]3O2)O1] | yes |
| I₁₀-DNYL-a | 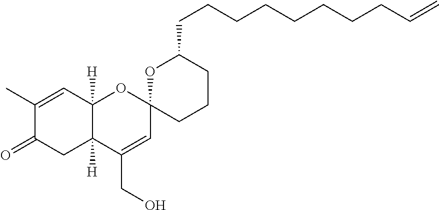 [SMILES: C=CCCCCCCCC[C@@H]1CCC[C@]2(C=C(CO)[C@H]3CC(=O)C(C)=C[C@H]3O2)O1] | na |
| I₁₀-Cit-a | 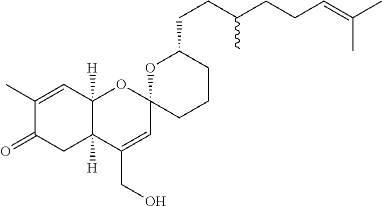 [SMILES: CC(C)=CCCC(C)CC[C@@H]1CCC[C@]2(C=C(CO)[C@H]3CC(=O)C(C)=C[C@H]3O2)O1] | na |
| I₁₀-H | 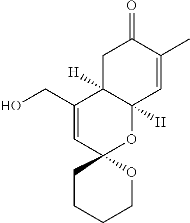 [SMILES: CC1=C[C@H]2O[C@]3(C=C(CO)[C@H]2CC1=O)CCCCO3] | no |
| I₁₀-Cit₃ | 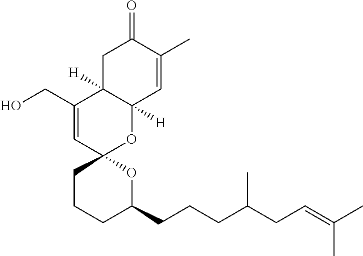 [SMILES: CC(C)=CCC(C)CCC[C@H]1CCC[C@]2(C=C(CO)[C@H]3CC(=O)C(C)=C[C@H]3O2)O1] | no |
| I₁₀-Deco₁ | 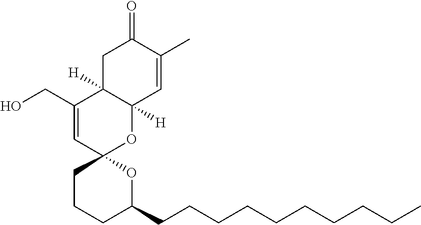 [SMILES: CCCCCCCCCC[C@H]1CCC[C@]2(C=C(CO)[C@H]3CC(=O)C(C)=C[C@H]3O2)O1] | no |
| I₁₀-DNYL-b | 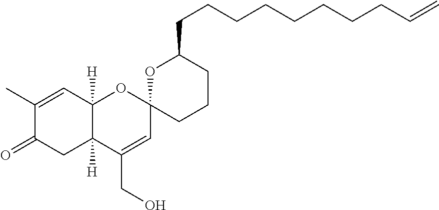 [SMILES: C=CCCCCCCCC[C@H]1CCC[C@]2(C=C(CO)[C@H]3CC(=O)C(C)=C[C@H]3O2)O1] | na |
| I₁₀-Cit-b | 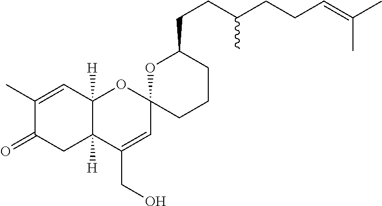 [SMILES: CC(C)=CCCC(C)CC[C@H]1CCC[C@]2(C=C(CO)[C@H]3CC(=O)C(C)=C[C@H]3O2)O1] | na |

na = not available

The disclosure may be further understood by the non-limiting examples. Although the description herein contains many specific examples, these should not be construed as limiting the scope of the disclosure but as merely providing illustrations of some of the embodiments of the disclosure. For example, thus the scope of the disclosure should be determined by the appended aspects and their equivalents, rather than by the examples given.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counter-ions those that are appropriate for preparation of salts of this disclosure for a given application. In specific applications, the selection of a given anion or cation for preparation of a salt may result in increased or decreased solubility of that salt. Every formulation or combination of components described or exemplified herein may be used to practice the disclosure, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Unless otherwise explained, all technical and scientific terms used herein have the same meaning as commonly understood by one of ordinary skill in the art to which a disclosed disclosure belongs.

### REFERENCES

Ashhurst, A. S. et al. 2021. Potent anti-SARS-CoV-2 activity by the natural product gallinamide A and analogues via inhibition of cathepsin L. J Med Chem. 2022 Feb. 24; 65(4):2956-2970. doi: 10.1021/acs.jmedchem.1c01494.
Auth, J. et al. 2021. Lectin from triticum vulgaris (WGA) inhibits infection with SARS-CoV-2 and its variants of concern alpha and beta. Int J Mol Sci. 2021 Sep. 22; 22(19):10205. doi: 10.3390/ijms221910205.
Bao, R. et al. 2018. Phellopterin-induced caspase-dependent apoptosis through PI3K/AKT pathway inhibition in SMMC-7721 human hepatoma cells. Am. J. Pharm. 37, 2498-2501.
Beans, E. J. et al. 2013 Highly potent, synthetically accessible prostratin analogs induce latent HIV expression in vitro and ex vivo. Proc Natl Acad Sci USA. 2013 Jul. 16; 110(29):11698-703. doi: 10.1073/pnas.1302634110. Epub 2013 Jun. 28.
Cagno, V. 2020. SARS-CoV-2 cellular tropism. Lancet Microbe. 2020 1(1):e2-e3. doi: 10.1016/S2666-5247(20)30008-2.
COVID-19—COVID-19 dashboard by the center for systems science and engineering (CSSE) at Johns Hopkins university (Dong, E. et al. Lancet Infect Dis. 2022 22(12):e370-e376. doi: 10.1016/S1473-3099(22)00434-0.).
Cox, R. M. et al. 2018 Development of an allosteric inhibitor class blocking RNA elongation by the respiratory syncytial virus polymerase complex J Biol Chem. 2018 Oct. 26; 293(43):16761-16777. doi: 10.1074/jbc.RA118.004862.
Chaiyosang, B. et al. 2015 A new lumazine peptide penilumamide E from the fungus Aspergillus terreus. Nat Prod Res. 2016 30(9):1017-24. doi: 10.1080/14786419.2015.1101107.
Chu, H. et al. 2020. Comparative tropism, replication kinetics, and cell damage profiling of SARS-CoV-2 and SARS-CoV with implications for clinical manifestations, transmissibility, and laboratory studies of COVID-19: an observational study. Lancet Microbe 1, e14-e23.
Daoust, J. et al. 2013. Sesterterpenoids isolated from a northeastern pacific phorbas sp. J. Org. Chem. 78, 8267-8273.
Ana Flávia Costa da Silveira Oliveira et al. 2017 Potential Antivirals: Natural Products Targeting Replication Enzymes of Dengue and Chikungunya Viruses. Molecules. 2017 Mar. 22; 22(3):505. doi: 10.3390/molecules22030505.
Alfonso Gennaro, 20th ed., Lippencott Williams & Wilkins, (2000)
Guo, J. et al. 2020 Screening of Natural Extracts for Inhibitors against Japanese Encephalitis Virus Infection. Antimicrob Agents Chemother. 2020 Feb. 21; 64(3):e02373-19. doi: 10.1128/AAC.02373-19.
Hacisuleyman, E. et al. 2021. Vaccine breakthrough infections with SARS-CoV-2 variants. N. Engl. J. Med. NEJMoa, 2105000.
Hoffmann, M. et al. 2021 SARS-CoV-2 variants B.1.351 and P.1 escape from neutralizing antibodies. Cell. 2021 Apr. 29; 184(9):2384-2393.e12. doi: 10.1016/j.cell.2021.03.036.
Khumkomkhet, P. et al. 2009. Antimalarial and cytotoxic depsidones from the fungus Chaetomium brasiliense. J. Nat. Prod. 72, 1487-1491.
Leary, E. et al. 2018 Quantitative Live-Cell Confocal Imaging of 3D Spheroids in a High-Throughput Format. SLAS Technol. 2018 June; 23(3):231-242. doi: 10.1177/2472630318756058. Epub 2018 Feb. 7. PMID: 29412762
Lee, S. Y. et al. 2018 3D Cell-Based High-Content Screening (HCS) Using a Micropillar and Microwell Chip Platform. Anal Chem. 2018 Jul. 17; 90(14):8354-8361. doi: 10.1021/acs.analchem.7b05328. Epub 2018 Jun. 29. PMID: 29889500
Lei, X. et al. 2020 Activation and evasion of type I interferon responses by SARS-CoV-2. Nat Commun. 2020 Jul. 30; 11(1):3810. doi: 10.1038/s41467-020-17665-9. PMID: 32733001
Mani, J. S. et al. 2020. Natural Product-Derived Phytochemicals as Potential Agents against Coronaviruses: A Review. Virus Res. 2020 Jul. 15; 284:197989.doi: 10.1016/j.virusres.2020.197989.
McKee, M. and Stuckler, D. 2020. If the world fails to protect the economy, COVID-19 will damage health not just now but also in the future. Nat Med. 2020 May; 26(5):640-642. doi: 10.1038/s41591-020-0863-y.
Miller, J. et al. 2023 Substantial Neutralization Escape by SARS-CoV-2 Omicron Variants BQ.1.1 and XBB.1. N Engl J Med. 2023 388(7):662-664. doi: 10.1056/NEJMc2214314.
Moghadamtousi, S. Z. et al. 2015. Potential antiviral agents from marine fungi: an overview. Mar. Drugs 13, 4520-4538.
Musarra-Pizzo, M. et al. 2021. Antiviral activity exerted by natural products against human viruses. Viruses 13.
Ogando, N. S. et al. 2020. SARS-coronavirus-2 replication in Vero E6 cells: replication kinetics, rapid adaptation and cytopathology. J. Gen. Virol. 101, 925.
Olmstead, A. D. et al. 2012. Human subtilase SKI-1/S1P is a master regulator of the HCV lifecycle and a potential host cell target for developing indirect-acting antiviral agents. PLoS Pathog. 8, e1002468.
Pérez-Vargas J. et al. 2023 Discovery of lead natural products for developing pan-SARS-CoV-2 therapeutics. Antiviral Res. 2023 209:105484. doi: 10.1016/j.antiviral.2022.105484. Epub 2022 Dec. 8.
Plante, J. A, et al. 2021 The variant gambit: COVID-19's next move. Cell Host Microbe. 2021 Apr. 14; 29(4):508-515. doi: 10.1016/j.chom.2021.02.020. Epub 2021 Mar. 1. PMID: 33789086.
Prosser, A. R. and Liotta, D. C. 2015 One-pot transformation of esters to analytically pure ketones: methodology and application in process development. Tetrahedron Letters 2015 56(23): 3005-3007.
Shapira, T. et al. 2022. A TMPRSS2 inhibitor acts as a pan-SARS-CoV-2 prophylactic and therapeutic. Nature 2022 605(7909):340-348.doi: 10.1038/s41586-022-04661-w. Epub 2022 Mar. 28.
Shen, L. et al. 2019 High-Throughput Screening and Identification of Potent Broad-Spectrum Inhibitors of Coronaviruses. J Virol. 2019 May 29; 93(12):e00023-19. doi: 10.1128/JVI.00023-19.
Vabret N. et al. 2020 Immunology of COVID-19: Current State of the Science. 2020 Immunity. 2020 Jun. 16; 52(6):910-941. doi: 10.1016/j.immuni.2020.05.002.
Wang, S. X. et al. 2014. Potential anti-HPV and related cancer agents from marine resources: an overview. Mar. Drugs 12, 2019-2035.
Wang, M. et al. 2016. Sesterterpenoids isolated from the sponge phorbas sp. activate latent HIV-1 provirus expression. J. Org. Chem. 81, 11324-11334.
Wibmer, C. K. et al. 2021 SARS-CoV-2 501Y.V2 escapes neutralization by South African COVID-19 donor plasma. Nat Med. 2021 27(4):622-625. doi: 10.1038/s41591-021-01285-x.
Wu, C.-J. et al. 2019. Terretonin D1, a new meroterpenoid from marine-derived Aspergillus terreus ML-44. Nat. Prod. Res. 33, 2262-2265.
Xie, X. et al. 2020. An infectious cDNA clone of SARS-CoV-2. Cell Host Microbe 27, 841-848.
Zorzitto, J. et al. 2006 Characterization of the antiviral effects of interferon-alpha against a SARS-like coronoavirus infection in vitro. Cell Res. 2006 16(2):220-9. doi: 10.1038/sj.cr.7310030.

1. A compound, the compound having the structure of Formula 1: 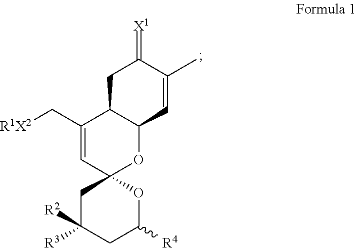
or pharmaceutical acceptable salt or prodrug thereof, for treatment of a coronavirus infection,
wherein:
X¹ is selected from: O; S; N—OH; and N—NH—R⁵;
X² is selected from: O; and S;
R¹ is selected from: H; and a one to ten carbon saturated, or unsaturated, linear, branched, or cyclic alkyl group, where individual carbon atoms are optionally substituted with OH, OR, ═O, NH, SH, F, Cl, Br, or are optionally replaced by N, or S;
R² and R³ are independently selected from: H; R; OR; or R₂ and R₃ jointly form ═CR⁶R⁷;
R⁴ is selected from: H; and a one to twenty carbon saturated, unsaturated, or aromatic, linear, branched, or cyclic alkyl group, where individual carbon atoms are optionally substituted with OH, OR, ═O, NH, SH, F, Cl, Br, or are optionally replaced by N, or S;
R⁵ is selected from: H; and a one to ten carbon saturated, or unsaturated, linear, branched, or cyclic alkyl group, where individual carbon atoms are optionally substituted with OH, OR, ═O, NH, SH, F, Cl, Br, or are optionally replaced by N, or S;
R⁶ is selected from: H; and a one to ten carbon saturated, or unsaturated, linear, branched, or cyclic, alkyl group where individual carbon atoms are optionally substituted with OH, OR, ═O, NH, SH, F, Cl, Br, or are optionally replaced by N, or S;
R⁷ is selected from: H; and a one to ten carbon saturated, or unsaturated, linear, branched, or cyclic alkyl group where individual carbon atoms are optionally substituted with OH, OR, ═O, NH, SH, F, Cl, Br, or are optionally replaced by N, or S; and
R is selected from: C(═O)CH₃; C(═O)CH₂CH(CH₃)CH₃; H; and CH₃.

2. The compound of claim 1, wherein the structure of Formula 1 is

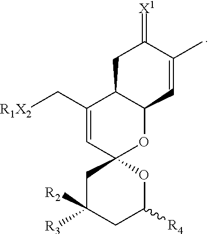

3. The compound of claim 1 or 2, wherein R⁴ is a saturated, unsaturated, linear or branched C8 to C12 alkyl group.

4. The compound of claim 1 or 2, wherein R⁴ is selected from:
H;

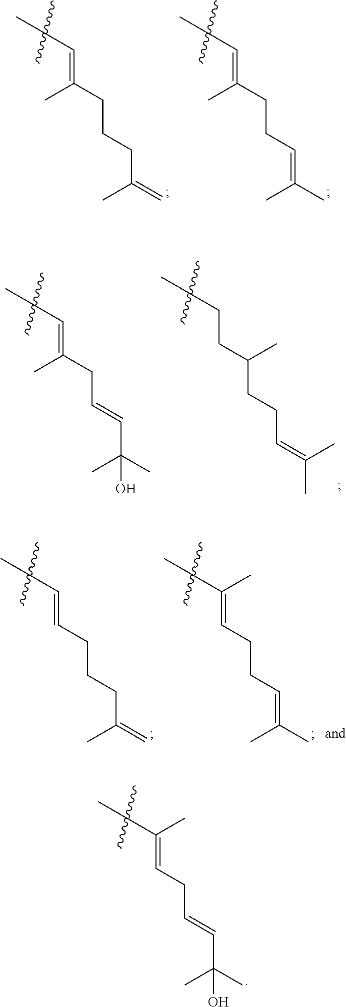

5. The compound of claim 1, 2 or 4, wherein R⁴ is selected from:
H;

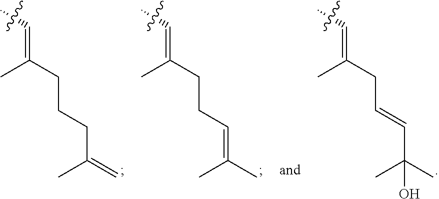

6. The compound of claim 1 or 2, wherein R⁴ is selected from: H;

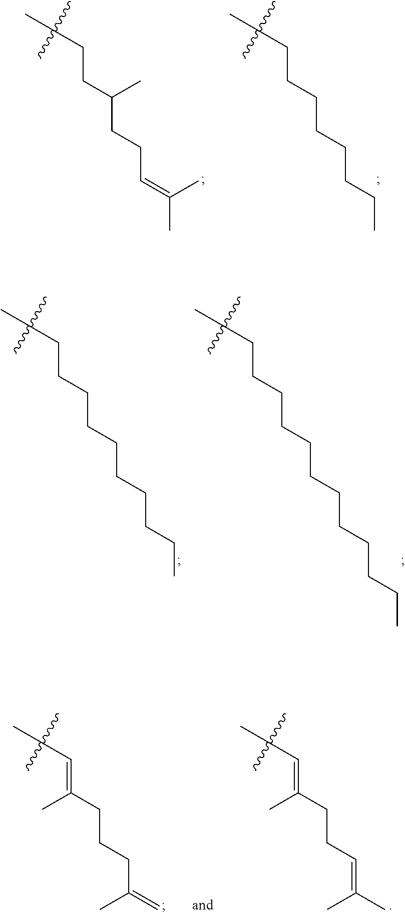

7. The compound of claim 1, 2 or 6, wherein R⁴ is selected from: H;

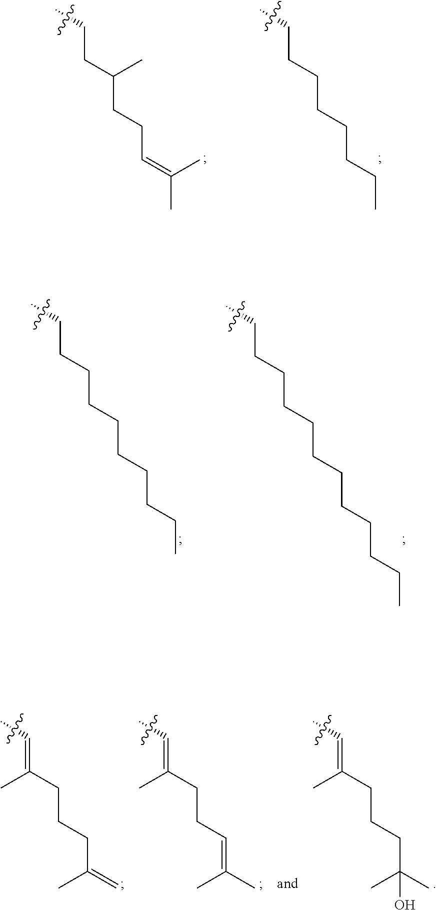

8. The compound of any one of claims 1-7, wherein R² and R³ are independently selected from: H; OC(═O)CH₃; OC(═O)CH₂CH(CH₃)CH₃; and OH; or jointly are ═CH₂.

9. The compound of any one of claims 1-8, wherein X¹ is selected from: O; and S; and X² is O.

10. The compound of any one of claims 1-9, wherein R¹ is H.

11. The compound of any one of claims 1-9, wherein the compound has the structure of Formula 2: 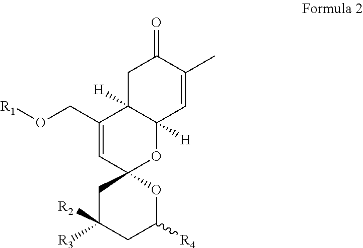
wherein:
R¹ is selected from: H; and CH₃C(O);
R² is OC(═O)CH₃; and R₃ is CH₃; or R² and R³ jointly form ═CH₂;
R⁴ is selected from: H; and a one to twenty carbon saturated, unsaturated, or aromatic, linear, branched, or cyclic alkyl group where individual carbon atoms may be substituted with OH, OR, ═O, NH, SH, F, Cl, Br, or be replaced by N, or S; and
R is selected from: C(═O)CH₃; C(═O)CH₂CH(CH₃)CH₃; H; and CH₃.

12. The compound of claim 11, wherein the structure of Formula 2 is

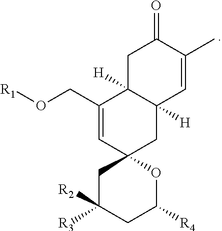

13. The compound of claim 1 or 2, wherein R⁴ is a saturated, unsaturated, linear or branched C8 to C12 alkyl group.

14. The compound of claim 11 or 12, wherein R⁴ is selected from:
H;

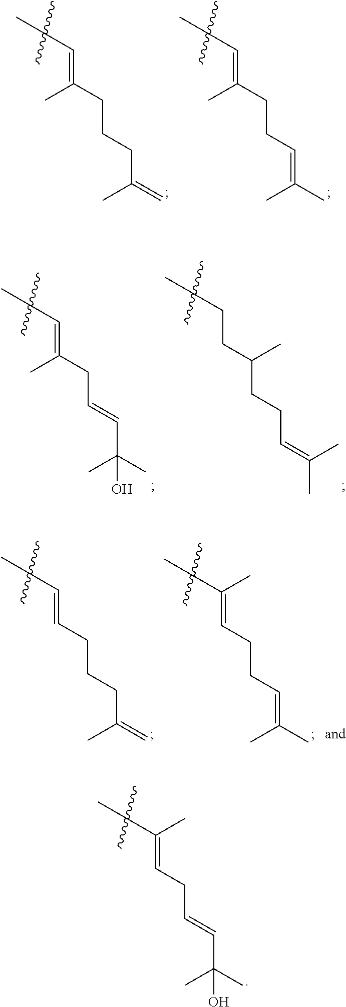

15. The compound of claim 11, 12 or 14, wherein R⁴ is selected from:
H;

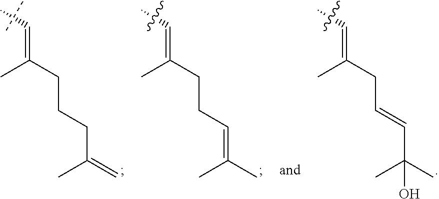

16. The compound of claim 11 or 12, wherein R⁴ is selected from: H;

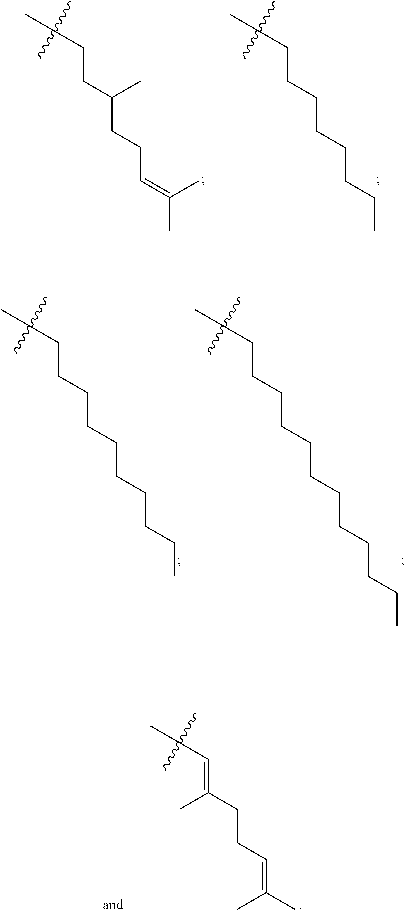

17. The compound of claim 11, 12 or 16, wherein R⁴ is selected from: H;

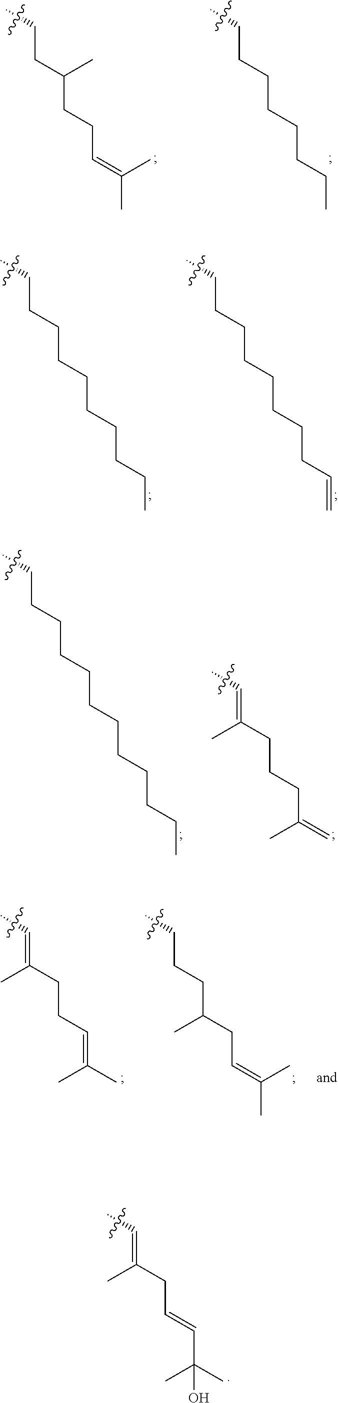

18. The compound of any one of claims 11-17, wherein R² and R³ are independently selected from: H; OC(═O)CH₃; OC(═O)CH₂CH(CH₃)CH₃; and OH; or jointly are ═CH₂.

19. The compound of any one of claims 11-18, wherein X¹ is selected from: O; and S; and X² is O.

20. The compound of any one of claims 11-19, wherein R¹ is H.

21. The compound of any one of claims 1-10, wherein the compound is selected from one or more of the following:

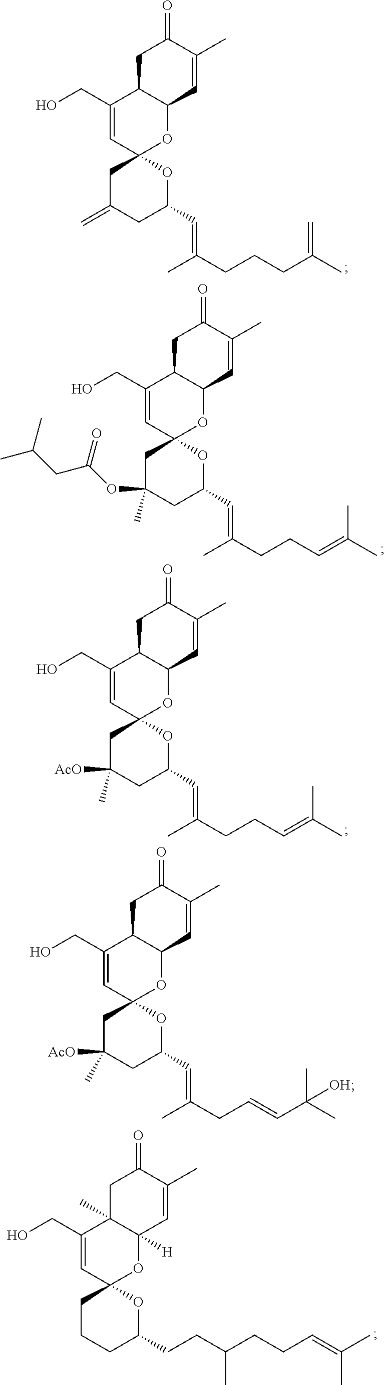

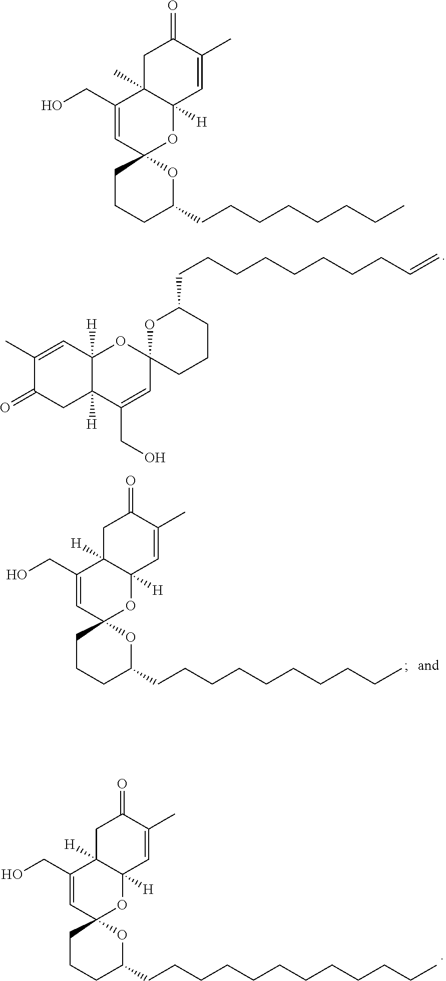

22. The compound of any one of claims 1-10, wherein the compound is selected from one or more of the following:

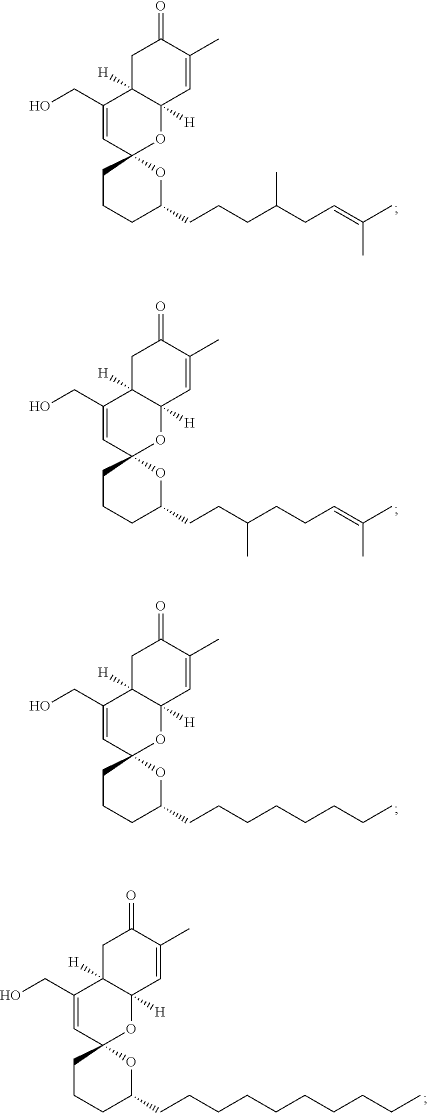 [SMILES: CC(C)=CCC(C)CCC[C@@H]1CCC[C@]2(C=C(CO)[C@H]3CC(=O)C(C)=C[C@H]3O2)O1 ; CC(C)=CCCC(C)CC[C@@H]1CCC[C@]2(C=C(CO)[C@H]3CC(=O)C(C)=C[C@H]3O2)O1 ; CCCCCCCC[C@@H]1CCC[C@]2(C=C(CO)[C@H]3CC(=O)C(C)=C[C@H]3O2)O1 ; CCCCCCCCCC[C@@H]1CCC[C@]2(C=C(CO)[C@H]3CC(=O)C(C)=C[C@H]3O2)O1]

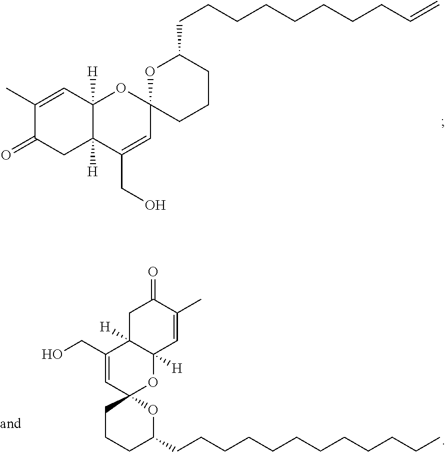 [SMILES: C=CCCCCCCCC[C@@H]1CCC[C@]2(C=C(CO)[C@H]3CC(=O)C(C)=C[C@H]3O2)O1 ; CCCCCCCCCCCC[C@@H]1CCC[C@]2(C=C(CO)[C@H]3CC(=O)C(C)=C[C@H]3O2)O1]

23. The compound of any one of claims 1-10, wherein the compound is selected from one or more of the following:

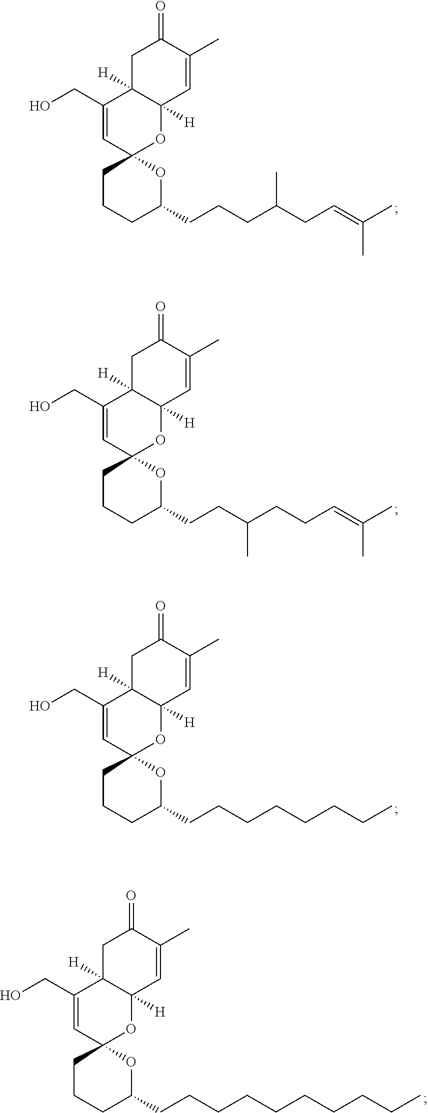 [SMILES: CC(C)=CCC(C)CCC[C@@H]1CCC[C@]2(C=C(CO)[C@H]3CC(=O)C(C)=C[C@H]3O2)O1 ; CC(C)=CCCC(C)CC[C@@H]1CCC[C@]2(C=C(CO)[C@H]3CC(=O)C(C)=C[C@H]3O2)O1 ; CCCCCCCC[C@@H]1CCC[C@]2(C=C(CO)[C@H]3CC(=O)C(C)=C[C@H]3O2)O1 ; CCCCCCCCCC[C@@H]1CCC[C@]2(C=C(CO)[C@H]3CC(=O)C(C)=C[C@H]3O2)O1]

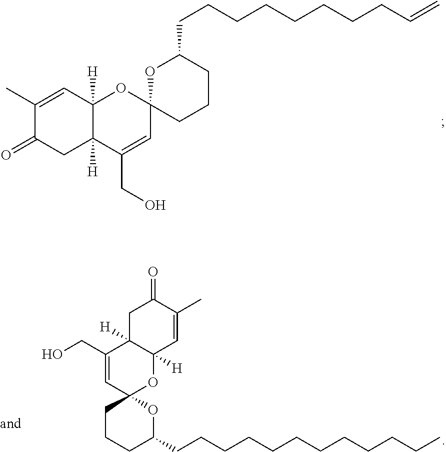 [SMILES: C=CCCCCCCCC[C@@H]1CCC[C@]2(C=C(CO)[C@H]3CC(=O)C(C)=C[C@H]3O2)O1 ; CCCCCCCCCCCC[C@@H]1CCC[C@]2(C=C(CO)[C@H]3CC(=O)C(C)=C[C@H]3O2)O1]

24. The compound of any one of claims 1-10, wherein the compound is selected from one or more of the following:

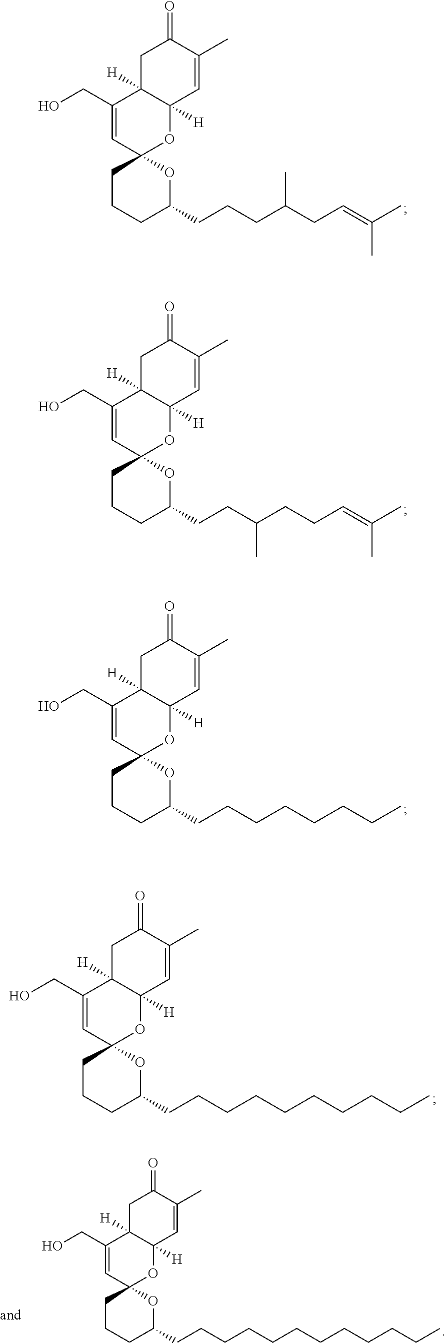 [SMILES: CC(C)=CCC(C)CCC[C@@H]1CCC[C@]2(C=C(CO)[C@H]3CC(=O)C(C)=C[C@H]3O2)O1 ; CC(C)=CCCC(C)CC[C@@H]1CCC[C@]2(C=C(CO)[C@H]3CC(=O)C(C)=C[C@H]3O2)O1 ; CCCCCCCC[C@@H]1CCC[C@]2(C=C(CO)[C@H]3CC(=O)C(C)=C[C@H]3O2)O1 ; CCCCCCCCCC[C@@H]1CCC[C@]2(C=C(CO)[C@H]3CC(=O)C(C)=C[C@H]3O2)O1 ; CCCCCCCCCCCC[C@@H]1CCC[C@]2(C=C(CO)[C@H]3CC(=O)C(C)=C[C@H]3O2)O1]

25. The compound of any one of claims 1-24, wherein the coronavirus infection is selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection.

26. The compound of any one of claims 1-25, wherein the coronavirus infection is a human coronavirus 229E (HCoV-229E) infection.

27. The compound of any one of claims 1-26, wherein the coronavirus infection is from a HCoV-229E variant selected from: Alpha; Beta; Gamma; Delta; and Omicron.

28. The compound of any one of claims 1-27, wherein the coronavirus infection is from a HCoV-229E variant selected from: Omicron and Delta.

29. The compound of any one of claims 1-28, wherein the coronavirus infection is from a HCoV-229E variant selected from: Omicron BA.1; Omicron BA.2; Omicron BA.5; or Delta B.1.617.2.

30. A compound, the compound having the structure of Formula 3: 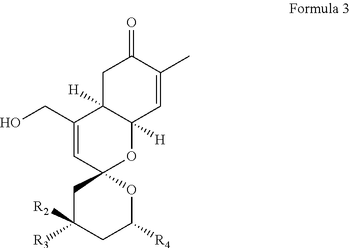
wherein:
R² is selected from: H; CH₃; OC(═O)CH₃; and OC(═O)CH₂CH(CH₃)CH₃;
R₃ is selected from: H; CH₃; OC(═O)CH₃; and OC(═O)CH₂CH(CH₃)CH₃;
alternatively, R² and R³ jointly form ═CH₂; and
R⁴ is a one to twenty carbon saturated, unsaturated, or aromatic, linear, branched, or cyclic alkyl group where individual carbon atoms may be substituted with OH, ═O, NH, SH, F, Cl, Br, or be replaced by N, or S;
provided that the compound excludes the following:

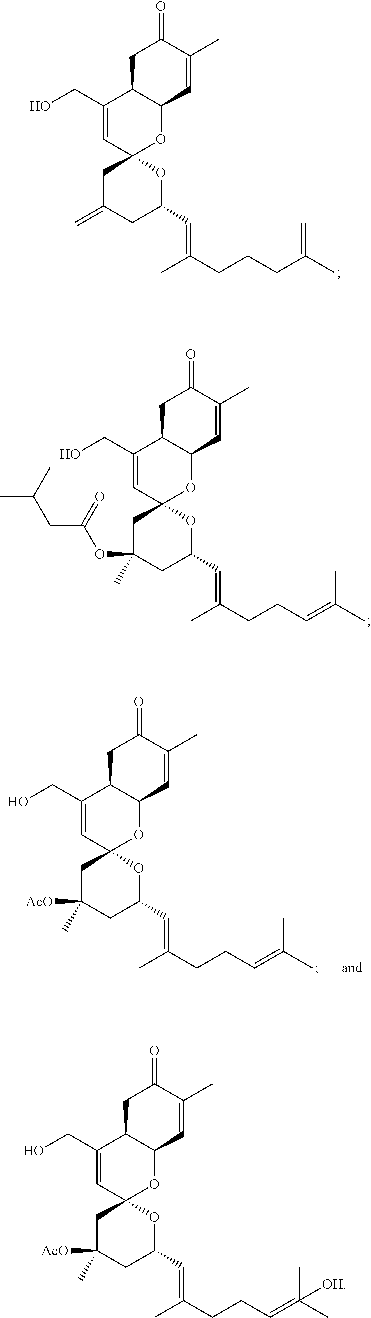

31. The compound of claim 30, wherein R⁴ is a one to twenty carbon saturated, unsaturated, linear, or branched alkyl group.

32. The compound of claim 30 or 31, wherein R⁴ is a one to twenty carbon saturated, unsaturated, linear, or branched alkyl group, where the individual carbon atoms may be substituted with OH, ═O, NH, SH, F, Cl, and Br.

33. The compound of claim 30, 31, or 32, wherein R⁴ is a one to twenty carbon saturated, unsaturated, linear, or branched alkyl group, where the individual carbon atoms may be substituted with OH, NH, F, Cl, and Br.

34. The compound of any one of claims 30-33, wherein R⁴ is a one to twenty carbon saturated, unsaturated, linear, or branched alkyl group, where the individual carbon atoms may be substituted with OH.

35. The compound of any one of claims 30-34, wherein R⁴ is a one to twenty carbon saturated, unsaturated, linear, or branched alkyl group, where the individual carbon atoms are unsubstituted.

36. The compound of any one of claims 30-35, wherein R² is selected from: H; OC(═O)CH₃; and OC(═O)CH₂CH(CH₃)CH₃; and R₃ is selected from: H; and CH₃.

37. The compound of any one of claims 30-36, wherein R² is H and R₃ is H.

38. The compound of any one of claims 30-37, wherein the compound is selected from:

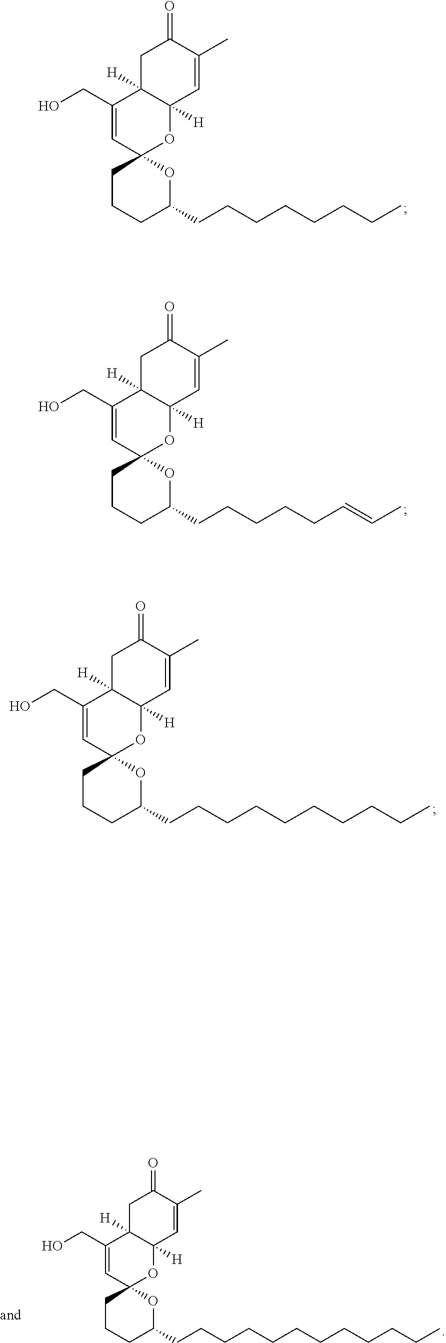 [SMILES: CCCCCCCC[C@@H]1CCC[C@]2(C=C(CO)[C@H]3CC(=O)C(C)=C[C@H]3O2)O1 ; C/C=C/CCCCC[C@@H]1CCC[C@]2(C=C(CO)[C@H]3CC(=O)C(C)=C[C@H]3O2)O1 ; CCCCCCCCCC[C@@H]1CCC[C@]2(C=C(CO)[C@H]3CC(=O)C(C)=C[C@H]3O2)O1 ; CCCCCCCCCCCC[C@@H]1CCC[C@]2(C=C(CO)[C@H]3CC(=O)C(C)=C[C@H]3O2)O1]

39. A method of treating a coronavirus infection in a subject in need thereof, the method comprising administration of a compound to the subject, wherein the compound is a compound of any one of claims 1-38, or pharmaceutical acceptable salt or prodrug thereof.

40. A pharmaceutical composition, the pharmaceutical composition comprising a compound of any one of claims 1-38, or pharmaceutical acceptable salt or prodrug thereof, for treatment of a coronavirus infection.

41. A pharmaceutical composition, the pharmaceutical composition comprising (a) a compound of any one of claims 30-38 or pharmaceutical acceptable salt or prodrug thereof, and (b) a pharmaceutically acceptable carrier.

42. Use of a compound of any one of claims 1-38, or pharmaceutical acceptable salt or prodrug thereof, or a pharmaceutical composition comprising the compound of any one of claims 1-38, for treating a coronavirus infection

43. Use of a compound of any one of claims 1-38, or pharmaceutical acceptable salt or prodrug thereof, or a pharmaceutical composition comprising the compound of any one of claims 1-38, in the manufacture of a medicament for treating a coronavirus infection.
